(19) United States
(12) Patent Application Publication
(10) Pub. No.: US 2025/0376460 A1
(43) Pub. Date: Dec. 11, 2025

(54) CB1R RECEPTOR BLOCKERS WITH ACYCLIC BACKBONES

(71) Applicant: YISSUM RESEARCH DEVELOPMENT COMPANY OF THE HEBREW UNIVERSITY OF JERUSALEM LTD, Jerusalem (IL)

(72) Inventors: Simon Benita, Tel Aviv (IL); Taher Nassar, Kfar Tur'an (IL); Joseph Tam, Jerusalem (IL); Shira Hirsh, Jerusalem (IL)

(21) Appl. No.: 19/170,919

(22) Filed: Apr. 4, 2025

Related U.S. Application Data
(63) Continuation of application No. 17/422,733, filed on Jul. 13, 2021.
(60) Provisional application No. 62/792,531, filed on Jan. 15, 2019.
(60) Provisional application No. 62/936,819, filed on Nov. 18, 2019.
(60) Provisional application No. 62/942,383, filed on Dec. 2, 2019.

(51) Int. Cl. C07D 401/12; A61P 3/04; A61P 3/10; C07D 231/14; C07D 473/00; C07D 491/044

(52) U.S. Cl. CPC C07D 401/12; A61P 3/04; A61P 3/10; C07D 231/14; C07D 473/00; C07D 491/044

(57) ABSTRACT

The invention generally concerns a novel class of CB1 receptor binding molecules and uses thereof.

### RELATED APPLICATIONS

This application is a continuation of and claims the benefit of U.S. patent application Ser. No. 17/422,733 filed Jul. 13, 2021, pending (now allowed), which is a National Phase Entry of PCT International Application No. PCT/IL2020/050062 which was filed on Jan. 15, 2020, which claims priority to U.S. Provisional Patent Application No. 62/792,531, filed Jan. 15, 2019, U.S. Provisional Patent Application No. 62/936,819, filed Nov. 18, 2019, and U.S. Provisional Patent Application No. 62/942,383, filed Dec. 2, 2019, all of which are hereby incorporated by reference in their entirety.

### TECHNOLOGICAL FIELD

The invention generally concerns novel peripherally restricted CB1 receptor blockers and uses thereof.

### BACKGROUND OF THE INVENTION

Obesity is a chronic disease reaching epidemic proportions, with more than one-third (34.9% or 78.6 million) of U.S. adults considered obese. Obesity has been described as a catalyst for a number of conditions, most notably cardiovascular disease, type 2 diabetes mellitus (T2DM) and non-alcoholic fatty liver disease (NAFLD). While several metabolic factors have been linked to the development of obesity, the molecular mechanisms involved in metabolism are not fully understood.

Endocannabinoids (eCBs) are endogenous lipid ligands that interact with the same cannabinoid receptors, CB1 and CB2, which also recognize Δ⁹-tetrahydrocannabinol (THC), the psychoactive component of Cannabis and mediate its biological effects. By activating CB1 receptors, eCBs increase appetite (the ‘munchies’) and lipogenesis in adipose tissue and liver and induce insulin resistance and dyslipidemia. These effects suggest that an overactive eCB/CB1 receptor system contributes to the development of visceral obesity, T2DM and their complications. Accordingly, this has prompted pharmaceutical companies to develop drugs that block CB1 receptors as potential treatment for obesity, T2DM and NAFLD. The first such compound, rimonabant [globally-acting CB1 receptor antagonist (1^{st} generation)], was effective not only in reducing body weight in obese and overweight individuals, but also in ameliorating the associated metabolic abnormalities, including fatty liver, insulin resistance and T2DM [1-6]. However, due to neuropsychiatric side effects (such as, depression, anxiety and suicidal ideation) rimonabant was withdrawn from the market worldwide, and CB1 receptors are no longer considered as a valid therapeutic target for obesity, T2DM or NAFLD.

### REFERENCES

[1] Van Gaal, L. F., Rissanen, A. M., Scheen, A. J., Ziegler, O. & Rossner, S. Effects of the cannabinoid-1 receptor blocker rimonabant on weight reduction and cardiovascular risk factors in overweight patients: 1-year experience from the RIO-Europe study. Lancet 365, 1389-1397 (2005).
[2] Pi-Sunyer, F. X., Aronne, L. J., Heshmati, H. M., Devin, J. & Rosenstock, J. Effect of rimonabant, a cannabinoid-1 receptor blocker, on weight and cardiometabolic risk factors in overweight or obese patients: RIO-North America: a randomized controlled trial. JAMA 295, 761-775 (2006).
[3] Despres, J. P., Golay, A., Sjostrom, L. & Rimonabant in Obesity-Lipids Study, G. Effects of rimonabant on metabolic risk factors in overweight patients with dyslipidemia. N Engl J Med 353, 2121-2134 (2005).
[4] Wierzbicki, A. S., et al. Rimonabant improves cholesterol, insulin resistance and markers of non-alcoholic fatty liver in morbidly obese patients: a retrospective cohort study. Int J Clin Pract 65, 713-715 (2011).
[5] Hollander, P. Endocannabinoid blockade for improving glycemic control and lipids in patients with type 2 diabetes mellitus. The American journal of medicine 120, S18-28; discussion S29-32 (2007).
[6] Randall, M. D., Kendall, D. A., Bennett, A. J. & O'Sullivan, S. E. Rimonabant in obese patients with type 2 diabetes. Lancet 369, 555 (2007).
[7] US 2003/0199536

### BRIEF SUMMARY OF THE INVENTION

The inventors of the technology disclosed herein have developed a methodology whereby peripherally restricted CB1 receptor antagonists retain the therapeutic benefits of globally acting CB1 receptor blockers without causing CNS-mediated side effects; thus, reviving the earlier prospect of CB1 receptor blockade for the treatment of metabolic syndromes. To that end, the inventors have designed a new class of novel compounds that do not penetrate the blood-brain-barrier and thus block the CB1 receptor only in peripheral organs, such as the adipose tissue, the liver, in skeletal muscles, pancreatic β-cells and the kidneys, without causing centrally-mediated side effects.

The inventors have also demonstrated that lipophilic compounds that bind a CB1 receptor and which are P-gp substrates; and/or have a brain/plasma ratio below 0.3; and/or have a diphenyl ethylene or diphenyl methylene moiety exhibit therapeutic benefits without causing CNS-mediated side effects.

This novel class of compounds exhibited efficacy in affecting several features of the metabolic syndrome.

Thus, in a first aspect of the presently disclosed invention, there is provided a lipophilic derivative of cannabinoid having a calculated Log P (partition coefficient between n-octanol and water) value ranging from 3 and 17.

The invention further provides a CB1 receptor-binding lipophilic compound, wherein:
the compound is a P-glycoprotein (P-gp) substrate; and/or
the compound has a brain/plasma ratio below 0.3; and/or
the compound having a diphenyl ethylene or diphenyl methylene moiety of formula (A):

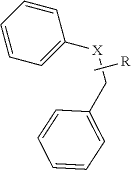

wherein
R is a substituent or a ring structure as defined in any of the structures below, X is a carbon containing group (C, C═, CH), a nitrogen containing group (N, N═, NH) or is absent; provided that R is different from H.

In a compound of formula (A), each of the phenyl groups, independently of the other, may or may not be substituted by 1, 2, 3, 4 or 5 same or different substituents.

In some embodiments, the CB1 receptor-binding lipophilic compound is a P-gp substrate.

In some embodiments, the CB1 receptor-binding lipophilic compound has a brain/plasma ratio below 0.3.

In some embodiments, the CB1 receptor-binding lipophilic compound comprises a diphenyl ethylene or diphenyl methylene moiety of formula (A), which may optionally be any of the compounds of general formulae (I) through (XXXXI) or any of the compounds specifically disclosed.

As indicated, compounds of the invention exhibit therapeutic benefits without causing CNS-mediated side effects. The absence of a CNS-mediated side effects is due, inter alia, to an interaction between compounds of the invention and P-gp (thus regarded as “P-gp substrates”) which limits or diminishes their penetration to the brain. The absence of or the diminished penetration to the brain may be qualitatively and, in some instances, quantitatively determined by means known in the art.

The brain-plasma concentration ratio representing one of the tools available for estimation of CNS pharmacokinetics is a parameter that indicates the blood-brain barrier availability of compounds. This value describes the free drug concentration of a compound in the brain, which is believed to be the parameter that causes the relevant pharmacological response at the target site. As indicated, compounds of the invention have exhibited substantially no brain penetration. Within the context of this aspect of the invention, the expression “substantially no brain penetration” refers no brain penetration to a brain-plasma ratio ranging from 0.0001 and 0.3. Compounds of the invention are further characterized by comprising a diphenyl ethylene or diphenyl methylene moiety of formula (A), as defined herein. In some embodiments, the compound of formula (A) is a compound of formula (I), as disclosed herein.

The invention further provides a lipophilic CB1 receptor-binding compound having a calculated Log P (partition coefficient between n-octanol and water) value ranging from 3 and 17, wherein the compound comprising a diphenyl ethylene or diphenyl methylene moiety of formula (A), as defined herein, or is a compound of formula (I), as disclosed herein.

The invention further provides a compound of formula (I):

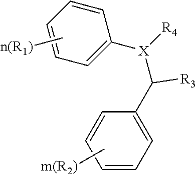

wherein
each of R₁ and R₂, independently of the other, is a group selected from —H, halide, —CN, —C₁-C₅alkyl-OH and —OH;
each of n and m, independently of the other, is an integer between 0 and 5, designating the number of substituents on the ring;
X is selected from nitrogen and —CH—; or X—R₄ may optionally be N═R₄ or C═R₄;
R₃ is selected from H, a carbon containing group comprising between 1 and 3 carbon atoms, being optionally substituted, and a nitrogen atom or a nitrogen containing group;
R₄ is selected from a carbon containing group comprising between 1 and 3 carbon atoms, being optionally substituted, and a nitrogen atom or a nitrogen containing group;
or R₃ and R₄ together with atoms to which they are bonded (carbon atom and X, respectively) form a 5- or 6-membered carbocyclic ring optionally containing between 1 and 3 heteroatoms selected from N, O and S;
or R₃ and R₄ together with the atoms to which they are bonded form a fused ring system optionally containing between 1 and 6 heteroatoms selected from N, O and S.

In some embodiments, X is N.

In some embodiments, X—R₄ is C═R₄.

In some embodiments, X—R₄ is N═R₄.

In some embodiments, X is a nitrogen atom and R₄ is a nitrogen containing group. In such embodiments, moiety X—R₄ may thus be selected from —N—NH—, —N═N— and —N—N═ (wherein in the selection the N on the left is X and the N on the right is R₄).

In some embodiments, R₃ is a carbon containing group and R₄ is a nitrogen containing group.

In some embodiments, R₃ and R₄ together with the atoms to which they are bonded form a 6-membered carbocyclic ring optionally containing 1 or 2 nitrogen atoms.

In some embodiments, R₃ and R₄ together with the atoms to which they are bonded form a 5-membered carbocyclic ring optionally containing 1 or 2 nitrogen atoms.

In some embodiments, R₃ and R₄ together with the atoms to which they are bonded form a fused ring system optionally containing 1, 2, 3, 4, 5, or 6 heteroatoms such as nitrogen atoms.

In some embodiments, the fused ring system is a two-ring fused system comprising a 5-membered ring that is fused to a 5-membered ring, or fused to a 6-membered ring, or fused to a 7-membered ring, or fused to a 8-membered ring. In some embodiments, the fused ring system is a two-ring fused system comprising a 5-membered ring that is fused to a 6-membered ring, wherein the fused system comprises 1, 2, 3, 4, or 5 heteroatoms. The fused system may further be substituted.

In some embodiments, the compound is of the general formula (II):

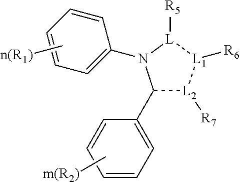

wherein
one of L, L₁ and L₂ is a nitrogen atom and the others of L, L₁ and L₂ are each a carbon atom (being selected from C, CH or CH₂);
each of R₅, R₆ and R₇, independently of the other, may be selected from —H, —C₁-C₃alkyl, —C(═O)—OH, —C(═O)—O—R₈, —C(═O)—NR′R₈, halide, —CN, —OH, and —NR′R″; or
one of R₅ and R₆ or R₆ and R₇ together with the atoms to which they bond may form a 5-, 6-, 7- or 8-membered carbocyclic ring optionally containing between 1 and 3 heteroatoms selected from N, O and S;
the 5-, 6-, 7- or 8-membered carbocyclic ring is further optionally substituted by at least one functionality selected from —H, —C₁-C₂₅alkyl, —C₂-C₂₅alkenyl, —C₂-C₂₅alkynyl, —C₆-C₁₀aryl, an hydroxyl, an amine, a halide, —ONO₂, —NO₂, —S—, —S—C₁-C₅alkyl, —S—C₂-C₅alkenyl, —S—C₂-C₅alkynyl, —C(═O)—, —C(═O)—C₁-C₂₅alkyl, —C(═O)—O—C₁-C₅alkyl, —C(═O)—O—C₂-C₅alkenyl, —C(═O)—O—C₂-C₅alkynyl, —C(═O)—NR′R″R′″, —C(═O)—NR′—C(═O)—C₁-C₂₅alkyl, —C(═O)—NR′—C(═O)—C₂-C₂₅alkenyl, —C(═O)—NR′—C(═O)—C₂-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″;
the 5-, 6-, 7- or 8-membered carbocyclic ring may be optionally substituted by at least one functionality selected from structures (A) through (H):

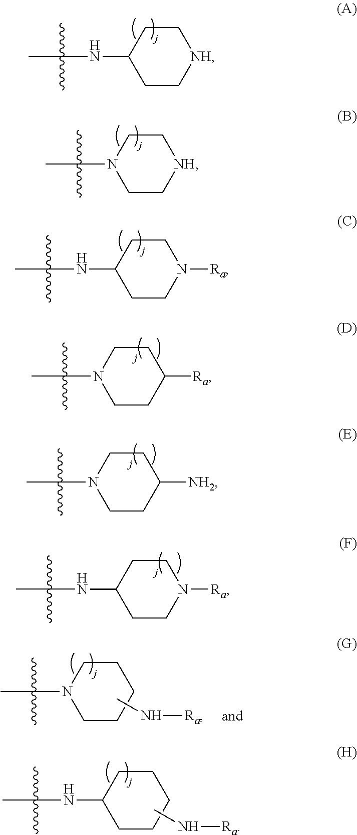

wherein in each functionality (A) through (H), the wavy line indicates point or bond of connectivity, j is 0 or 1 and Ra is selected from —H, —C₁-C₂₅alkyl, —C₂-C₂₅alkenyl, —C₂-C₂₅alkynyl, —C(═O)—C₆-C₁₀aryl and —C(═O)—C₃-C₁₀heteroaryl,
wherein in functionalities (G) and (H) the pendant —NH—Ra group may appear between 1 and 11 times at any position along the carbocycle (in some embodiments, it may be positioned at a ring atom once removed, twice removed or three times removed from the existing group or endocyclic N atom; in some embodiments, the position of the functionality is 1, 2 or 1, 3 or 1,4, wherein 1 designates the position of the existing group or the endocyclic N atom);
one of R₅, R₆ and R₇ may be absent;
R₈ is selected from —H, —C₁-C₂₅alkyl, —C₂-C₂₅alkenyl, —C₂-C₂₅alkynyl, —C₆-C₁₀aryl and C₃-C₁₀heteroaryl, each of which being optionally substituted by at least one functionality selected from an hydroxyl, an amine, a halide, —ONO₂, —NO₂, —S—, —S—C₁-C₅alkyl, —S—C₂-C₅alkenyl, —S—C₂-C₅alkynyl, —C(═O)—, —C(═O)—C₁-C₂₅alkyl, —C(═O)—O—C₁-C₅alkyl, —C(═O)—O—C₂-C₅alkenyl, —C(═O)—O—C₂-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″;
R₁₀ is selected from —H, —C₁-C₂₅alkyl, —C₂-C₂₅alkenyl, —C₂-C₂₅alkynyl, —C₆-C₁₀aryl, each of which being optionally substituted by at least one functionality selected from an hydroxyl, an amine, a halide, —C₁-C₅alkyl, —C₂-C₅alkenyl, —C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-aryl-Cl, idebenonyl-derivative, -pyridine-3-C(═O)—OH and —NR′R″R′″;
each of R′, R″ and R′″ is independently selected from —H, C₁-C₅alkyl, C₂-C₅alkenyl, C₂-C₅alkynyl, —C(═O)—C₂-C₂₅alkyl, —C(═O)—C₂-C₂₅alkenyl and C₅-C₂₅alkynyl;
or wherein one of R′, R″ and R′″ is absent; and wherein each bond between N-L, L-L₁, L₁-L₂ and L₂-C (designated ---) is a single or double bond.

In some embodiments, R₈ is —C₁-C₂₅alkyl.

In some embodiments, R₈ is —C₂-C₂₅alkenyl.

In some embodiments, R₈ is —C₂-C₂₅alkynyl.

In some embodiments, R₈ is —C₆-C₁₀aryl.

In some embodiments, R₈ is C₃-C₁₀heteroaryl.

In some embodiments, R₈ is —C₁-C₂₅alkyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

In some embodiments, R₈ is —C₂-C₂₅alkenyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

In some embodiments, R₈ is —C₂-C₂₅alkynyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

In some embodiments, R₈ is —C₆-C₁₀aryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

In some embodiments, R₈ is C₃-C₁₀heteroaryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

In some embodiments, in all compounds of the invention, excluded are compounds wherein R₈ is C₇-C₁₂alkyl. In such cases, where R₈ is said to be an alkyl having between 1 and 25 carbon atoms (inclusive), namely an alkyl of the form C₁-C₂₅alkyl or C₁-C₂₅alkylene, in consideration of the aforementioned exclusion, the alkyl or alkylene may be stated to be C₁-C₆alkyl/alkylene and C₁₃-C₂₅alkyl/alkylene.

In some embodiments, the 5-, 6-, 7- or 8-membered carbocyclic ring substituted by at least one functionality selected from structures (A) through (H):

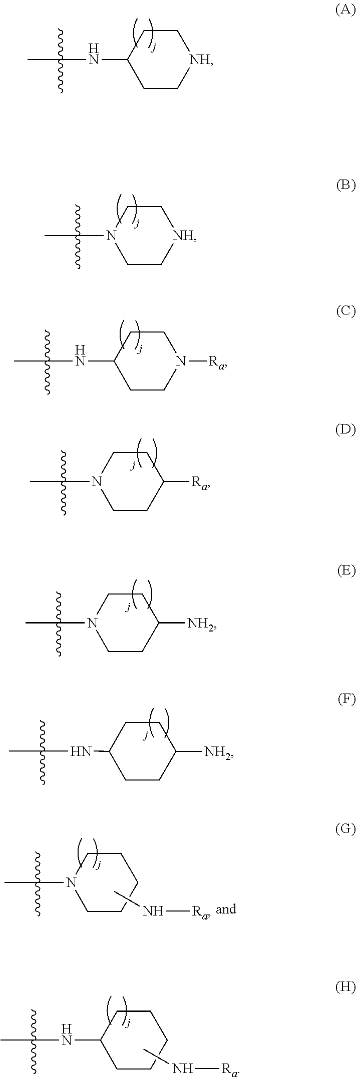

In some embodiments, in each functionality (A) through (H), j is 0.

In some embodiments, in each functionality (A) through (H), j is 1.

In some embodiments, the pendant —NH—Ra group appears once. In some embodiments, —NH—Ra is positioned at a ring atom once removed from the existing group or endocyclic N atom. In some embodiments, the —NH—Ra is positioned at a ring atom twice removed from the existing group or endocyclic N atom. In some embodiments, the —NH—Ra is positioned at a ring atom three times removed from the existing group or endocyclic N atom.

The invention further provides a compound of formula (II), as defined herein.

As used herein, a “carbon containing group having between 1 and 3 carbon atoms” is any carbon chain or carbon-containing group or a carbon-containing functionality that comprises one to three carbon atoms, inclusive, which may be bonded to each other or may be separated or interrupted by one or more atoms that are not carbon. In some embodiments, the carbon containing group is a group comprising a chain of one to three carbon atoms, each of which being connected to another atom. Non-limiting examples of such carbon groups include —CH, —CH₂—, —CH₃, —CH—CH—, —CH₂—CH—, —CH═CH—, —CH—CH₂—, —CH₂—CH₂—CH₂—, —CH₂—CH—CH—, —CH₂—CH═CH— and others. Non-limiting examples of such carbon groups that include one or more atoms that are not carbon, e.g., a heteroatom such as nitrogen, include —CH—NH—, —C═N—, —CH₂—NH—, —N—CH₃, —CH—NH—CH—, —CH₂—CH—NH—, —CH═N—CH—, —CH—NH—CH₂—, —CH₂—NH—CH₂—CH₂—, —CH₂—CH—NH—CH—, —CH₂—CH═CH—NH—, —CH₂—N═CH—NH—, —CH₂—NH—CH═CH—NH—, —CH₂—N═CH—, and others. Such groups may be optionally substituted.

The carbon-containing group containing between 1 and 3 carbon atoms may be alternatively designated as —C₁-C₃alkyl, —C₂-C₃alkenyl or —C₂-C₃alkynyl, or any substituted for thereof.

A “nitrogen atom or a nitrogen-containing group” is similarly any group of atoms or a functionality that comprises one or more nitrogen atoms. The nitrogen(s) atom may be substituted with hydrogen atoms or with a carbon group or any other functionality. In some embodiments, the nitrogen containing group is a group such as —NH—, —NH₂—, —NHR′, NH₂R′, NHR′R″, NR′R″R′″, wherein each of R′, R″ and R′″ is as further defined herein. The nitrogen containing group may additionally be selected from nitrogen-containing cycles. Non-limiting examples of such nitrogen-containing cycles include aziridinyl, azetidinyl, pyrrolidinyl, Imidazolidinyl, imidazolyl, Pyrazolidinyl, Pyrazolyl, triazolyl, piperidinyl, pyridinyl, piperazinyl, diazinyl, triazinyl, trihydrotriazinyl, indolyl, isoindolyl, quinolinyl, isoquinolinyl and others. The nitrogen atom or nitrogen-containing group may be presented in a form of a charged nitrogen atom (an ammonium).

As disclosed herein, e.g., with reference to variables R₃ and R₄, any two groups, as recited, together with atoms to which they are bonded (carbon atom and X, respectively, when in reference to variables R₃ and R₄) may form a 5- or 6-membered carbocyclic ring optionally containing a heteroatom, e.g., between 1 and 3 heteroatoms, inclusive, wherein the heteroatoms may be selected from N, O and S. Other non-carbon atoms may also be present. The 5- or 6-membered ring comprises one or more carbon atoms in a cyclic form (forming a carbocyclic structure). The carbon chain forming the carbocycle may be interrupted by one or more heteroatoms, together forming a heterocyclic ring structure.

In some embodiments, the heterocyclic ring may comprise 1, 2 or 3 nitrogen atoms. In some embodiments, the heterocyclic ring may comprise 1, 2 or 3 oxygen atoms. In some embodiments, the heterocyclic ring may comprise 1, 2 or 3 sulfur atoms.

In some embodiments, the heterocyclic ring may comprise 1, 2 or 3 nitrogen and/or oxygen and/or sulfur atoms.

In some embodiments, the heterocyclic ring may comprise 1 or 2 nitrogen atoms.

Alternatively, variables R₃ and R₄ together with atoms to which they are bonded (carbon atom and X, respectively, when in reference to variables R₃ and R₄) may form a fused ring system as defined.

R₈ is selected from —H, —C₁-C₂₅alkyl, —C₂-C₂₅alkenyl, —C₂-C₂₅alkynyl, —C₆-C₁₀aryl and —C₃-C₁₀heteroaryl. As used herein with reference to R₈ or to any other variable, the alkyl, alkenyl and alkynyl are each as known in the art.

Where R₈ or any other group is a C₁-C₂₅alkyl, it may be linear, branched or cyclic and may optionally be substituted by one or more substituents as defined. In some embodiments, R₈ is a linear alkyl comprising a number of carbon atoms selected from between 1 and 25, 1 and 20, 1 and 10, 5 and 25, 5 and 20, 10 and 25, 10 and 20, 15 and 25, 15 and 20 or between 20 and 25 carbon atoms. In some embodiments, the linear alkyl comprises 1, 2, 3, 4, 5, 6, 7, 8, 9, 10, 11, 12, 13, 14, 15, 16, 17, 18, 19 or 20 carbon atoms. In some embodiments, the linear alkyl comprises 6, 10, 16 or 18 carbon atoms.

Where the alkyl group is substituted on both ends, it may be regarded as an alkylene group.

In some embodiments, the alkyl group is a non-linear, branched or cyclic —C₅-C₂₅alkyl.

Where R₈ or any other group is a C₅-C₂₅alkenyl, it may be linear, branched or cyclic and comprising one or more double bonds in cis or trans configuration. The double bond may be a mid-chain double bond or a terminal double bond. Where R₈ is a cyclic alkenyl, the double bond may be endocyclic or exocyclic. In some embodiments, R₈ is a linear alkenyl comprising a number of carbon atoms selected from between 5 and 25, 5 and 20, 5 and 10, 10 and 25, 10 and 20, 15 and 25, 15 and 20 or between 20 and 25 carbon atoms. In some embodiments, the linear alkenyl comprises 5, 6, 7, 8, 9, 10, 11, 12, 13, 14, 15, 16, 17, 18, 19 or 20 carbon atoms. In some embodiments, the linear alkenyl comprises between 1 and 10 double bonds, each double bond may independently be in a cis or trans configuration. Where the alkenyl group is substituted on both ends, it may be regarded as an alkenylene group. Where R₈ or any other group is a C₅-C₂₅alkynyl, it may be linear, branched or cyclic and comprising one or more triple bonds. The triple bond may be a mid-chain bond or a terminal bond. Where R₈ is a cyclic alkynyl, the triple bond may be endocyclic or exocyclic. In some embodiments, R₈ is a linear alkynyl comprising a number of carbon atoms selected from between 5 and 25, 5 and 20, 5 and 10, 10 and 25, 10 and 20, 15 and 25, 15 and 20 or between 20 and 25 carbon atoms. In some embodiments, the linear alkynyl comprises 5, 6, 7, 8, 9, 10, 11, 12, 13, 14, 15, 16, 17, 18, 19 or 20 carbon atoms. In some embodiments, the linear alkynyl comprises between 1 and 5 triple bonds. Where the alkynyl group is substituted on both ends, it may be regarded as an alkynylene group.

In some embodiments, the alkyl, alkenyl or alkynyl may be selected from CH₃(CH₂)₃—, CH₃(CH₂)₄—, CH₃(CH₂)₅—, CH₃(CH₂)₆—, CH₃(CH₂)₇—, CH₃(CH₂)₈—, CH₃(CH₂)₉—, CH₃(CH₂)₁₀—, CH₃(CH₂)₁₁—, CH₃(CH₂)₁₂—, CH₃(CH₂)₁₃—, CH₃(CH₂)₁₄—, CH₃(CH₂)₁₅—, CH₃(CH₂)₁₆—, CH₃(CH₂)₁₇—, CH₃(CH₂)₁₈—, CH₃(CH₂)₁₉—, CH₃(CH₂)₂₀—, CH₃(CH₂)₂₁—, CH₃(CH₂)₂₂—, CH₃(CH₂)₂₃—, (CH₃)₂CHCH₂—, CH₃(CH₂)₃CH═CH(CH₂)₇—, CH₃(CH₂)₅CH═CH(CH₂)₇—, CH₃(CH₂)₈CH═CH(CH₂)₄—, CH₃(CH₂)₇CH═CH(CH₂)₇—, CH₃(CH₂)₇CH═CH(CH₂)₇—, CH₃(CH₂)₅CH═CH(CH₂)₉—, CH₃(CH₂)₄CH═CHCH₂CH═CH(CH₂)₇—, CH₃(CH₂)₄CH═CHCH₂CH═CH(CH₂)₇—, CH₃CH₂CH═CHCH₂CH═CHCH₂CH═CH(CH₂)₇—, CH₃(CH═CH)₂—, CH₃(CH₂)₄CH═CHCH₂CH═CHCH₂—CH═CHCH₂CH═CH(CH₂)₃—, CH₃CH₂CH═CHCH₂CH═CHCH₂CH═CHCH₂CH═CHCH₂—CH═CH(CH₂)₃—, CH₃(CH₂)₇CH═CH(CH₂)₁₁—, CH₃CH₂CH═CHCH₂CH═CHCH₂CH═CH—CH₂CH═CHCH₂CH═CHCH₂CH═CH(CH₂)₂—, CH₃CH₂CH═CHCH₂CH═CHCH₂CH═CH—CH₂CH═CH(CH₂)₄—, CH₃(CH₂)₄CH═CHCH₂CH═CHCH₂CH═CH(CH₂)₄—, CH₃(CH₂)₄CH═CHCH₂CH═CHCH₂CH═CH(CH₂)₆—, CH₃(CH₂)₄CH═CHCH₂CH═CHCH₂CH═CHCH₂—CH═CH(CH₂)₅—, CH₃(CH₂)₅CH═CH(CH₂)₁₁—, CH₃(CH₂)₇CH═CH(CH₂)₉—, CH₃(CH₂)₇CH═CH(CH₂)₁₃—, CH₃(CH₂)₇CH═CHCH₂CH═CHCH₂CH═CH(CH₂)₃—, C₆H₅CH═CH—, CH₃(CH₂)₃C≡C(CH₂)₇—, CH₃(CH₂)₅C≡C(CH₂)₇—, CH₃(CH₂)₈C≡C(CH₂)₄—, CH₃(CH₂)₇C≡C—(CH₂)₇—, CH₃(CH₂)₇C≡C(CH₂)₇—, CH₃(CH₂)₅C≡C(CH₂)₉—, CH₃(CH₂)₄C≡CCH₂CH═CH(CH₂)₇—, CH₃(CH₂)₄CH═CHCH₂C≡C(CH₂)₇—, CH₃(CH₂)₄C≡CCH₂C≡C(CH₂)₇—, CH₃CH₂C≡CCH₂CH═CHCH₂CH═CH(CH₂)₇—, CH₃(C≡C)₂—, CH₃(CH₂)₄C≡CCH₂CH═CHCH₂—CH═CHCH₂CH═CH(CH₂)₃—, CH₃(CH₂)₄CH═CHCH₂CH═CHCH₂C≡CCH₂CH═CH(CH₂)₃—, CH₃CH₂CH═CHCH₂CH═CHCH₂C≡CCH₂CH═CHCH₂—CH═CH(CH₂)₃—, CH₃(CH₂)₇C≡C(CH₂)₁₁—, CH₃CH₂C≡CCH₂CH═CHCH₂C≡CCH₂CH═CHCH₂C≡CCH₂—CH═CH(CH₂)₂—, CH₃CH₂CH═CHCH₂CH═CHCH₂C≡C—CH₂C≡C(CH₂)₄—, CH₃(CH₂)₄C≡CCH₂CH═CHCH₂C≡C(CH₂)₄—, CH₃(CH₂)₄CH═CHCH₂CH═CHCH₂C≡C(CH₂)₆—, CH₃(CH₂)₄C≡CCH₂CH═CHCH₂C≡CCH₂—CH═CH(CH₂)₅—, CH₃(CH₂)₅C≡C(CH₂)₁₁—, CH₃(CH₂)₇C≡C(CH₂)₉—, CH₃(CH₂)₇C≡C(CH₂)₁₃—, CH₃(CH₂)₇C≡CCH₂CH═CH—CH₂C≡C(CH₂)₃—C₆H₅C≡C— and alkenylene derived from DHA (all-cis-docosa-4,7,10,13,16,19-hexa-enoic acid).

Where R₈ or any other group is a C₆-C₁₀aryl, the aryl group, as known in the art, may be any aromatic system comprising between 6 and 10 atoms, typically carbon atoms. The aryl group may be a single aromatic ring, such as a phenyl or a benzyl ring; a group containing two or more rings structures, one or more of which being aromatic, such as a diphenyl group; or a fused ring system comprising at least one aromatic ring, such as fused phenyl rings and naphthyl groups.

Where R₈ or any other group is a C₃-C₁₀heteroaryl, the group comprises one or more heteroatom in the ring structure. Such groups may contain nitrogen oxygen or sulfur atoms as ring atoms. Non-limiting examples include pyrrolyl, pyridyl, pyrimidyl, pyrazinyl, indolyl, quinolyl, isoquinolyl, furyl, thienyl, oxazolyl, benzoxazolyl, thiazolyl, benzothiazolyl, benzofuranyl, benzdioxolyl, benzothiophenyl and others. Substitution of the heteroaryl group may be at any position, typically at any carbon atom of the heteroaryl group. For example, the pyridyl group may be substituted ortho, meta or para to the N atom.

In some embodiments, in a compound of formula (II), R₅ or R₆ or R₇ is —C(═O)—O—R₈ or —C(═O)—NR′R₈, and R₈ is —C₁-C₂₅alkyl selected, for example, from —(CH₂)₈CH═CH(CH₂)₇CH₃, —(CH₂)₂—, —(CH₂)₁₅CH₃, —(CH₂)₁₅CH₃ and —(CH₂)₂CH═CH(CH₂CH═CH)₅CH₂CH₃.

In some embodiments, in a compound of formula (II), R₆ or R₇ is —C(═O)—O—R₈ or —C(═O)—NR′R₈, and R₈ is selected from 2,2,6,6-tetramethylpiperidin-1-ol-4-yl, —NHC(═O)CH₂C(CH₃)₂—O-aryl-Cl, idebenonyl-derivative, -pyridine-3-C(═O)—OH and —NR′R″R′″.

The group “2,2,6,6-tetramethylpiperidin-1-ol-4-yl” is the radical having the structure:

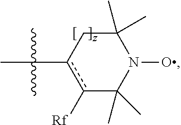

wherein z is 1, Rf is H and wherein the dashed bond is a single bond. Yet, also encompassed are groups wherein Z is zero, the dashed bond is a single bond or a double bond and wherein Rf is H or is selected from halide, —CN, —OH, —C₁-C₂₅alkyl, —C₂-C₂₅alkenyl, —C₂-C₂₅alkynyl, —C₆-C₁₀aryl, each of which being optionally substituted by at least one functionality selected from an hydroxyl, an amine, a halide, —C₁-C₅alkyl, —C₂-C₅alkenyl, —C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—OR₁₀, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, and —NR′R″R′″, as defined herein.

In some embodiments, in a group wherein Z is zero, the groups may be selected from:

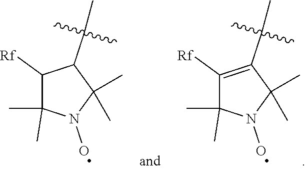

The group “—NHC(═O)CH₂C(CH₃)₂—O-aryl-Cl” designates a substituted aryl group, wherein the chloride atom and the ether group are substituted on the aryl structure ortho, meta or para to each other. In some embodiments, the group has the structure:

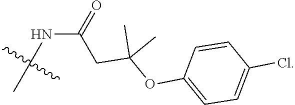 [SMILES: *NC(=O)CC(C)(C)Oc1ccc(Cl)cc1]

The “idebenonyl-derivative” is a group of the structure:

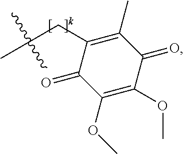

wherein k is an integer between 0 and 25.

In some embodiments, k is between 1 and 25, 1 and 20, 1 and 15, 1 and 10, 1 and 5, 5 and 25, 5 and 20, 5 and 10, 10 and 25 or between 10 and 20. In some embodiments, k is 5, 6, 7, 8, 9, 10, 11, 12, 13, 14 or 15. In some embodiments, k is 10.

The group “-pyridine-3-C(═O)—OH” is a niacin acid derivative, wherein the substitution on the pyridine ring may be at any position relative to the carboxylic acid group or to the ring nitrogen atom.

The group “—NR′R″R′″” designates an amine which may be a primary amine, a secondary amine, a tertiary amine or a quaternary amine. Each of the R groups may be selected as disclosed herein. In some embodiments, each of R′, R″ and R′″ is independently —H, —C₁-C₅alkyl, —C₂-C₅alkenyl, —C₂-C₅alkynyl, —C(═O)—C₂-C₂₅alkyl, —C(═O)—C₂-C₂₅alkenyl or C₅-C₂₅alkynyl. In cases where the group designates a charged nitrogen atom (an ammonium), the three R groups are presented and may be selected as indicated. In cases where the group designates an uncharged nitrogen atom, one of R′, R″ and R′″ is absent and the remaining two groups may be each selected as indicated herein.

As recited herein, in a compound of formula (II), R₅ or R₆ or R₇ may be —C(═O)—O—R₈ or —C(═O)—NR′R₈, wherein R₈ is selected as above. Each of the groups selected for R₈ may be substituted or unsubstituted. In some embodiments, the groups selected for R₈, namely —C₁-C₂₅alkyl, —C₂-C₂₅alkenyl, —C₂-C₂₅alkynyl and —C₆-C₁₀aryl, may be substituted by at least one functionality selected from an hydroxyl (—OH), an amine (primary, secondary, tertiary or quaternary amine), a halide (selected F, Br, Cl and I), —C₁-C₅alkyl, —C₂-C₅alkenyl, —C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—OR₁₀, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-aryl-Cl, idebenonyl-derivative, -pyridine-3-C(═O)—OH and —NR′R″R′″.

In some embodiments, R₅ or R₆ or R₇ is —C(═O)—O—R₈ and R₈ is selected as above. In some embodiments, R₈ is —C₁-C₂₅alkyl. In some embodiments, the —C₁-C₂₅alkyl is selected from optionally substituted —(CH₂)₈CH═CH(CH₂)₇CH₃, —(CH₂)₂—, —(CH₂)₁₅CH₃, —(CH₂)₁₅CH₃ and —(CH₂)₂CH═CH(CH₂CH═CH)₅CH₂CH₃. In some embodiments, the aforementioned groups are substituted by —NR′R″R′″, wherein one of said R′, R″ and R′″ is absent and the other of R′, R″ and R′″ is selected from —H, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″ and —C(═O)—OR₁₀, as defined herein. In some embodiments, the group —NR′R″R′″ is thus —NHR′″ (R′ absent and R″=H), wherein R′″ is —H, —C(═O)—, —C(═O)—O—C₁-C₅alkyl, —C(═O)—O—C₁-C₅alkenyl, —C(═O)—O—C₁-C₅alkynyl, —C(═O)—NR′R″R′″ or —C(═O)—OR₁₀. In some embodiments, R′″ is —C(═O)—, —C(═O)—C₁-C₂₅alkyl, —C(═O)—O—C₁-C₅alkyl, —C(═O)—O—C₁-C₅alkenyl, or —C(═O)—O—C₁-C₅alkynyl. In some embodiments, R′″ is —C(═O)— or —C(═O)—C₁-C₂₅alkyl.

In some embodiments, R₅ or R₆ or R₇ is —C(═O)—O—R₈ and R₈ is selected from —(CH₂)₈CH═CH(CH₂)₇CH₃, —(CH₂)₂—, —(CH₂)₁₅CH₃, —(CH₂)₁₅CH₃, —(CH₂)₂—NHC(═O)(CH₂)₇CH═CH(CH₂)₇CH₃ and —(CH₂)₂CH═CH(CH₂CH═CH)₅CH₂CH₃.

In some embodiments, R₅ or R₆ or R₇ is —C(═O)—O—(CH₂)₈CH═CH(CH₂)₇CH₃, —C(═O)—O—(CH₂)₂—, —C(═O)—O—(CH₂)₁₅CH₃, —C(═O)—O—(CH₂)₁₅CH₃, —C(═O)—O—(CH₂)₂—NHC(═O)(CH₂)₇CH═CH(CH₂)₇CH₃ and —C(═O)—O—(CH₂)₂CH═CH(CH₂CH═CH)₅—CH₂CH₃.

In some embodiments, R₅ or R₆ or R₇ is —C(═O)—NR′R₈ and R₈ is selected as above. In some embodiments, R₈ is —C₁-C₂₅alkyl. In some embodiments, the —C₁-C₂₅alkyl is selected from optionally substituted —(CH₂)₈CH═CH(CH₂)₇CH₃, —(CH₂)₂—, —(CH₂)₁₅CH₃, —(CH₂)₁₅CH₃ and —(CH₂)₂CH═CH(CH₂CH═CH)₅CH₂CH₃.

In some embodiments, R₅ or R₆ or R₇ is —C(═O)—NR′R₈ and R₈ is selected from —(CH₂)₈CH═CH(CH₂)₇CH₃, —(CH₂)₂—, —(CH₂)₁₅CH₃, —(CH₂)₁₅CH₃, —(CH₂)₂—NHC(═O)(CH₂)₇CH═CH(CH₂)₇CH₃ and —(CH₂)₂CH═CH(CH₂CH═CH)₅CH₂CH₃. In some embodiments, R′ is H.

In some embodiments, R₅ or R₆ or R₇ is —C(═O)—NH—(CH₂)₈CH═CH(CH₂)₇CH₃, —C(═O)—NH—(CH₂)₂—, —C(═O)—NH—(CH₂)₁₅CH₃, —C(═O)—NH—(CH₂)₁₅CH₃ and —C(═O)—NH—(CH₂)₂—NHC(═O)(CH₂)₇CH═CH(CH₂)₇CH₃ and —C(═O)—NH—(CH₂)₂CH═CH—(CH₂CH═CH)₅CH₂CH₃.

In some embodiments, in a compound of formula (II), L is a nitrogen atom (or a nitrogen containing group of atoms) and each of L₁ and L₂ is a carbon atom (or a carbon containing group of atoms).

In some embodiments, L is a nitrogen atom (or a nitrogen containing group of atoms), each of L₁ and L₂ is a carbon atom (or a carbon containing group of atoms), the bond between N and L is a single bond, the bond between L and L₁ is a double bond, and the bond between L₁ and L₂ is a single bond.

In some embodiments, R₅ is absent.

In some embodiments, the compound is of formula (III):

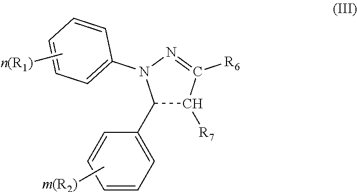

wherein each of R₁, R₂, n, m, R₆ and R₇ are as defined herein, and wherein --- designates a single or a double bond (in case it is a double bond, the carbon atom bearing variant R₇ does not carry a bond to a hydrogen atom).

As indicated herein, each of R₆ and R₇, independently of the other, may be selected from —H, —C₁-C₃alkyl, —C(═O)—OH, —C(═O)—O—R₈, —C(═O)—NR′R₈, halide, —CN, —OH, and —NR′R″; or

R₆ and R₇ together with the atoms to which they bond may form a 5-, 6-, 7- or 8-membered carbocyclic ring optionally containing between 1 and 3 heteroatoms selected from N, O and S. Substitution may be as indicated above.

In some embodiments, R₇ is H and R₆ is selected from —C₁-C₃alkyl, —C(═O)—O—R₈, —C(═O)—NR′—R₈, a halide, —CN, —OH, and —NR′R″; wherein R₈ is as defined herein.

In some embodiments, R₆ is —C(═O)—NR′R₈; and R₈ is as defined herein.

In some embodiments, R₆ is —C(═O)—NHR₈; and R₈ is as defined herein.

In some embodiments, the bond --- is a double bond.

In some embodiments, the bond --- is a single bond.

In some embodiments, the compound is of general formula (IV):

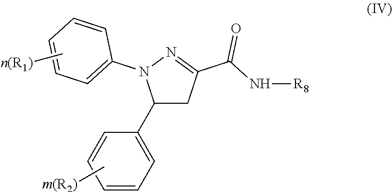

wherein each of R₁, R₂, n, m and R₈ is as defined herein.

In some embodiments, R₈ is a C₁-C₂₅alkyl, optionally substituted, as disclosed and selected herein.

In some embodiments, n is 2 and m is 1.

In some embodiments, R₁ and R₂ are each a halide.

In some embodiments, each of R₁ and R₂ is a chloride atom.

In some embodiments, the compound is of the formula (V):

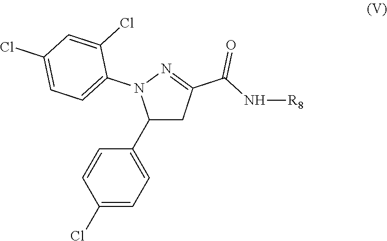

wherein R₈ is as defined herein.

In some embodiments, for a compound of formula (IV) and/or (V), R₈ may be:
—C₁-C₂₅alkyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₂-C₂₅alkenyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₂-C₂₅alkynyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₆-C₁₀aryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₃-C₁₀heteroaryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″.

In some embodiments, in a compound of formula (II), L is a nitrogen atom, each of L₁ and L₂ is a carbon atom, the bond between N and L is a single bond, the bond between L and L₁ is a double bond, the bond between L₁ and L₂ is a single bond and the bond between L₂ and C is a double bond.

In some embodiments, the compound is of the general formula (VI):

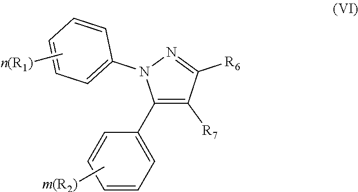

wherein each of R₁, R₂, n, m, R₆ and R₇ is as defined herein.

In some embodiments, R₆ is selected from —C₁-C₃alkyl, —C(═O)—O—R₈, —C(═O)—NR′—R₈, a halide, —CN, —OH, and —NR′R″;
R₇ is a C₁-C₃ alkyl;
R₈ is as defined herein.

In some embodiments, R₆ is —C(═O)—NR′—R₈; and R₈ is a C₁-C₂₅alkyl.

In some embodiments, the compound is of general formula (VII):

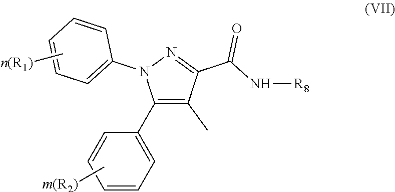

wherein each of R₁, R₂, n, m and R₈ is as defined herein.

In some embodiments, for a compound of formula (VII), R₈ may be:
—C₁-C₂₅alkyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₂-C₂₅alkenyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₂-C₂₅alkynyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₆-C₁₀aryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₃-C₁₀heteroaryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″.

In some embodiments, compounds of formulae herein (all formulae recited generically or specifically herein) exclude compounds wherein R₈ is C₇-C₁₂alkyl.

In some embodiments, R₈ is a C₁-C₂₅alkyl.

In some embodiments, R₈ is 2,2,6,6-tetramethylpiperidin-1-ol-4-yl.

In some embodiments, the compound is of the general formula (Vill):

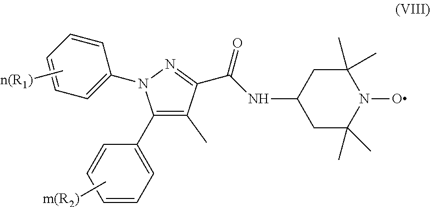

In some embodiments, n is 2 and m is 1.

In some embodiments, R₁ and R₂ are each a halide.

In some embodiments, each of R₁ and R₂ is a chloride atom.

In some embodiments, the compound is of the formula (IX):

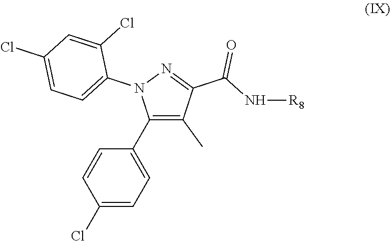

wherein R₈ is as defined herein.

In some embodiments, for a compound of formula (IX), R₈ may be:
—C₁-C₂₅alkyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″; or
—C₂-C₂₅alkenyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₂-C₂₅alkynyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₆-C₁₀aryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₃-C₁₀heteroaryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″.

In some embodiments, R₈ is 2,2,6,6-tetramethylpiperidin-1-ol-4-yl.

In some embodiments, the compound is of the formula (X):

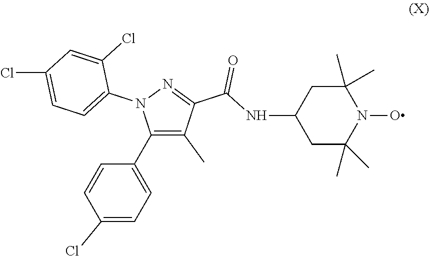

In some embodiments, in a compound of formula (I):
each of R₁ and R₂, independently of the other is a group selected from H, a halide and —CN;
each of n and m, independently of the other, is an integer between 0 and 5, designating the number of substituents on the ring;
X is selected from a nitrogen atom (or a nitrogen containing group) and CH;
or X—R₄ may optionally be C═R₄;
and
R₃ is H or a carbon containing group and R₄ is a nitrogen containing group.

In some embodiments, X is CH and R₄ is a carbon containing group having between 1 and 3 carbon atoms.

In some embodiments, R₃ is H.

In some embodiments, the compound is of the general formula (XI):

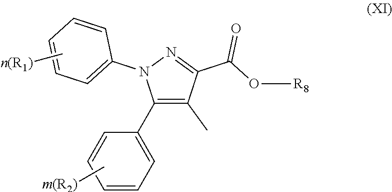

wherein each of R₁, R₂, n, m and R₈ is as defined herein, optionally excluding compounds wherein R₈ is C₇-C₁₂alkyl.

In some embodiments, for a compound of formula (XI), R₈ may be:
—C₁-C₂₅alkyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₂-C₂₅alkenyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₂-C₂₅alkynyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₆-C₁₀aryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₃-C₁₀heteroaryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″.

In some embodiments, R₈ is a C₁-C₂₅alkyl.

In some embodiments, R₈ is 2,2,6,6-tetramethylpiperidin-1-ol-4-yl.

In some embodiments, the compound is of the general formula (XII):

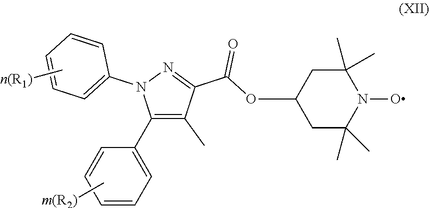

In some embodiments, in a compound of formula (XI), R₈ is an idebenonyl derivative.

In some embodiments, the compound is of the formula (XIII):

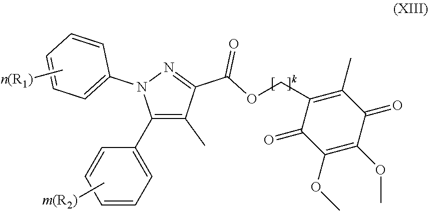

wherein each of R₁, R₂, n and m are as defined above and wherein k is an integer between 0 to 25.

In some embodiments, n is 2 and m is 1.

In some embodiments, R₁ and R₂ are each a halide.

In some embodiments, each of R₁ and R₂ is a chloride atom.

In some embodiments, the compound is of the general formula (XIV):

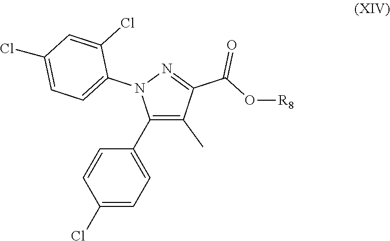

wherein R₈ is as defined herein.

In some embodiments, for a compound of formula (XIV), R₈ may be:
—C₁-C₂₅alkyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₂-C₂₅alkenyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₂-C₂₅alkynyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₆-C₁₀aryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″; or
—C₃-C₁₀heteroaryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, pyridine-3-C(═O)—OH and —NR′R″R′″.

In some embodiments, R₈ is 2,2,6,6-tetramethylpiperidin-1-ol-4-yl.

In some embodiments, the compound is of the formula (XV):

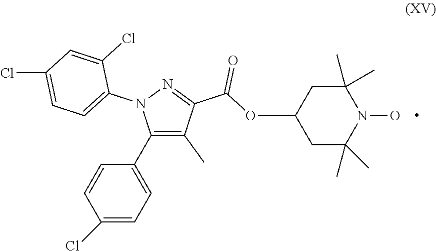 [SMILES: Cc1c(C(=O)OC2CC(C)(C)N(O)C(C)(C)C2)nn(-c2ccc(Cl)cc2Cl)c1-c1ccc(Cl)cc1]

In some embodiments, R₈ is an idebenonyl derivative.

In some embodiments, the compound is of the formula (XVI):

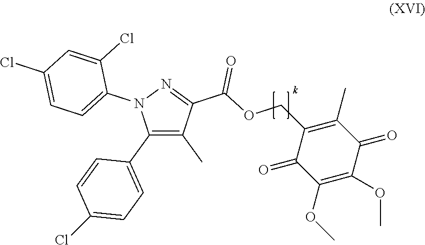

wherein k is as defined above.

In some embodiments, in a compound of formula (IV), R₈ is C₁-C₂₅alkyl optionally substituted by at least one functionality selected from —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—OR₁₀, wherein each of R′, R″, R′″ and R₁₀ is as defined above.

In some embodiments, the at least one functionality is selected from —C(═O)—NR′R″R′″, —C(═O)—C₁-C₂₅alkyl and —C(═O)—OR₁₀, wherein each of R′, R″, R′″ and R₁₀ is as defined above.

In some embodiments, R′ is H, R″ is absent and R′″ is R₁₁, wherein R₁₁ is selected from —H or a C₁-C₂₅alkyl, C₂-C₂₅alkenyl, C₂-C₂₅alkynyl, C₆-C₁₀aryl, each of which being optionally substituted by at least one functionality selected from an hydroxyl, an amine, a halide, C₁-C₅alkyl, C₂-C₅alkenyl, C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—OR₁₀, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-Aryl-Cl, idebenonyl-derivative, -pyridine-3-C(═O)—OH and —NR′R″R′″, and wherein R′, R″, R′″ and R₁₀ are as defined above.

In some embodiments, the compound is of the general formula (XVII):

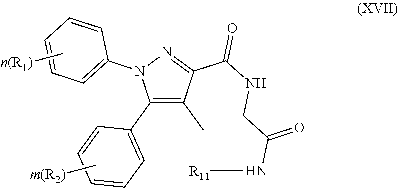

wherein each of R₁, R₂, n, m and R₁₁ is as defined herein.

In some embodiments, n is 2 and m is 1.

In some embodiments, R₁ and R₂ are each a halide.

In some embodiments, each of R₁ and R₂ is a chloride atom.

In some embodiments, the compound is of the general formula (XVIII):

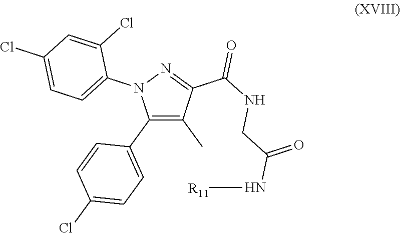

wherein R₁₁ is as defined herein.

In some embodiments, R₁₁ is selected from —H or a C₁-C₂₅alkyl, C₂-C₂₅alkenyl, C₂-C₂₅alkynyl, C₆-C₁₀aryl, each of which being optionally substituted by at least one functionality selected from an hydroxyl, an amine, a halide, C₁-C₅alkyl, C₂-C₅alkenyl, C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—OR₁₀, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-Aryl-Cl, idebenonyl-derivative, -pyridine-3-C(═O)—OH and —NR′R″R′″.

In some embodiments, in a compound of formula (XI), R₈ is —C₁-C₂₅alkyl optionally substituted by at least one functionality selected from —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—OR₁₀, wherein each of R′, R″, R′″ and R₁₀ is as defined above.

In some embodiments, the at least one functionality is selected from —C(═O)—NR′R″R′″, —C(═O)—C₁-C₂₅alkyl and —C(═O)—OR₁₀, wherein each of R′, R″, R′″ and R₁₀ is as defined above.

In some embodiments, the compound is of the general formula (XIX):

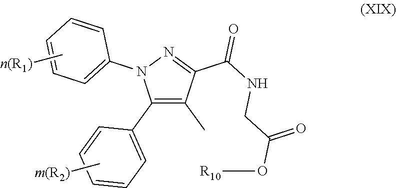

wherein each of R₁, R₂, n, m and R₁₀ is as defined herein.

In some embodiments, R₁₀ is selected from —H, —C₁-C₂₅alkyl, —C₂-C₂₅alkenyl, —C₂-C₂₅alkynyl, —C₆-C₁₀aryl, each of which being optionally substituted by at least one functionality selected from an hydroxyl, an amine, a halide, —C₁-C₅alkyl, —C₂-C₅alkenyl, —C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-aryl-Cl, idebenonyl-derivative, -pyridine-3-C(═O)—OH and —NR′R″R′″;
each of R′, R″ and R′″ is independently selected from —H, C₁-C₅alkyl, C₂-C₅alkenyl, C₂-C₅alkynyl, —C(═O)—C₂-C₂₅alkyl, —C(═O)—C₂-C₂₅alkenyl and C₅-C₂₅alkynyl; or wherein one of R′, R″ and R′″ is absent.

In some embodiments, n is 2 and m is 1.

In some embodiments, R₁ and R₂ are each a halide.

In some embodiments, each of R₁ and R₂ is a chloride atom.

In some embodiments, the compound is of the general formula (XX):

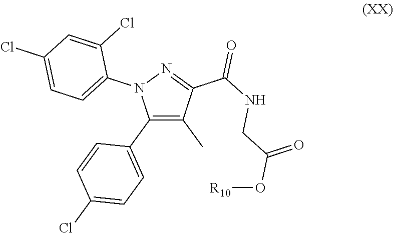

wherein R₁₀ is as defined herein.

In some embodiments, R₁₀ is selected from —H, —C₁-C₂₅alkyl, —C₂-C₂₅alkenyl, —C₂-C₂₅alkynyl, —C₆-C₁₀aryl, each of which being optionally substituted by at least one functionality selected from an hydroxyl, an amine, a halide, —C₁-C₅alkyl, —C₂-C₅alkenyl, —C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-aryl-Cl, idebenonyl-derivative, -pyridine-3-C(═O)—OH and —NR′R″R′″;
each of R′, R″ and R′″ is independently selected from —H, C₁-C₅alkyl, C₂-C₅alkenyl, C₂-C₅alkynyl, —C(═O)—C₂-C₂₅alkyl, —C(═O)—C₂-C₂₅alkenyl and C₅-C₂₅alkynyl; or wherein one of R′, R″ and R′″ is absent.

In some embodiments, in a compound of formula (IV), R₈ is C₁-C₂₅alkyl optionally substituted by at least one functionality selected from an hydroxyl, an amine, —OR₁₀, and a halide.

In some embodiments, the at least one functionality is a hydroxyl, an amine or —OR₁₀, wherein the amine having the structure —NR′R″R′″, wherein each of R′, R″, R′″ and R₁₀ is as defined above.

In some embodiments, R′ is H, R″ is absent and R′″ is R₁₁, wherein R₁₁ is selected from —H, a —C₁-C₂₅alkyl, —C₂-C₂₅alkenyl, —C₂-C₂₅alkynyl, —C₆-C₁₀aryl, each of which being optionally substituted by at least one functionality selected from an hydroxyl, an amine, a halide, C₁-C₅alkyl, C₂-C₅alkenyl, C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—OR₁₀, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-Aryl-Cl, idebenonyl-derivative, -pyridine-3-C(═O)—OH and —NR′R″R′″, and wherein R′, R″, R′″ and R₁₀ are as defined above.

In some embodiments, the compound is of the general formula (XXI):

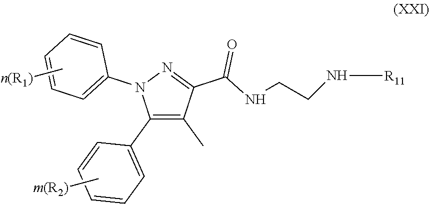

wherein each of R₁, R₂, n, m and R₁₁ is as defined herein.

In some embodiments, R₁₁ is selected from —H or a C₁-C₂₅alkyl, C₂-C₂₅alkenyl, C₂-C₂₅alkynyl, C₆-C₁₀aryl, each of which being optionally substituted by at least one functionality selected from an hydroxyl, an amine, a halide, C₁-C₅alkyl, C₂-C₅alkenyl, C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—OR₁₀, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-Aryl-Cl, idebenonyl-derivative, -pyridine-3-C(═O)—OH and —NR′R″R′″.

In some embodiments, R′ is H, R″ is absent and R′″ is R₁₁, wherein R₁₁ is —NHC(═O)CH₂C(CH₃)₂—O-Aryl-Cl.

In some embodiments, the compound is of the general formula (XXII):

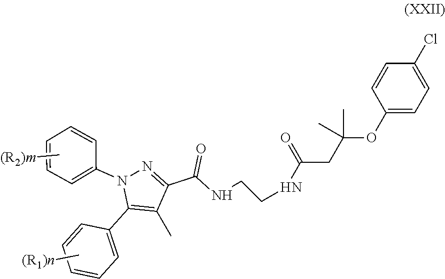

wherein each of R₁, R₂, n and m is as defined herein.

In some embodiments, n is 2 and m is 1.

In some embodiments, R₁ and R₂ are each a halide.

In some embodiments, each of R₁ and R₂ is a chloride atom.

In some embodiments, the compound is of the general formula (XXIII):

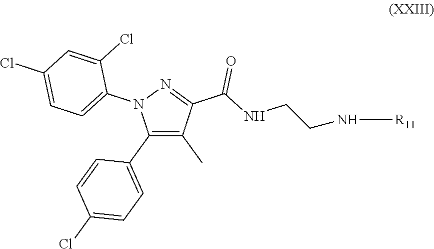

wherein R₁₁ is as defined herein.

In some embodiments, R₁₁ is selected from —H or a C₁-C₂₅alkyl, C₂-C₂₅alkenyl, C₂-C₂₅alkynyl, C₆-C₁₀aryl, each of which being optionally substituted by at least one functionality selected from an hydroxyl, an amine, a halide, C₁-C₅alkyl, C₂-C₅alkenyl, C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—OR₁₀, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-Aryl-Cl, idebenonyl-derivative, -pyridine-3-C(═O)—OH and —NR′R″R′″.

In some embodiments, the compound is of the formula (XXIV):

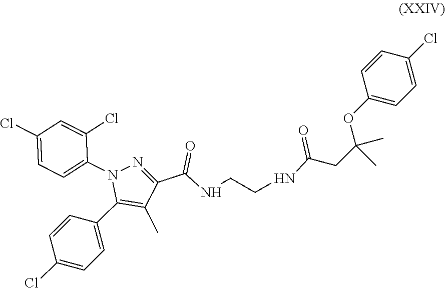 [SMILES: Cc1c(C(=O)NCCNC(=O)CC(C)(C)Oc2ccc(Cl)cc2)nn(-c2ccc(Cl)cc2Cl)c1-c1ccc(Cl)cc1]

In some embodiments, in a compound of formula (XI), R₈ is C₁-C₂₅alkyl optionally substituted by at least one functionality selected from an hydroxyl, an amine, —OR₁₀, and a halide.

In some embodiments, the at least one functionality is a hydroxyl, an amine or —OR₁₀, wherein the amine having the structure NR′R″R′″, wherein each of R′, R″, R′″ and R₁₀ is as defined above.

In some embodiments, the compound is of the general formula (XXV):

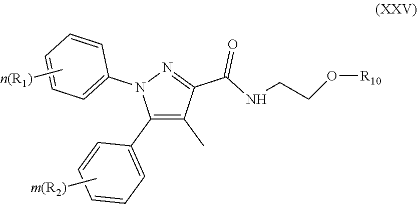

wherein each of R₁, R₂, n, m and R₁₀ is as defined herein.

In some embodiments, R₁₀ is selected from —H, —C₁-C₂₅alkyl, —C₂-C₂₅alkenyl, —C₂-C₂₅alkynyl, —C₆-C₁₀aryl, each of which being optionally substituted by at least one functionality selected from an hydroxyl, an amine, a halide, —C₁-C₅alkyl, —C₂-C₅alkenyl, —C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-aryl-Cl, idebenonyl-derivative, -pyridine-3-C(═O)—OH and —NR′R″R′″;
each of R′, R″ and R′″ is independently selected from —H, C₁-C₅alkyl, C₂-C₅alkenyl, C₂-C₅alkynyl, —C(═O)—C₂-C₂₅alkyl, —C(═O)—C₂-C₂₅alkenyl and C₅-C₂₅alkynyl; or wherein one of R′, R″ and R′″ is absent.

In some embodiments, n is 2 and m is 1.

In some embodiments, R₁ and R₂ are each a halide.

In some embodiments, each of R₁ and R₂ is a chloride atom.

In some embodiments, the compound is of the general formula (XXVI):

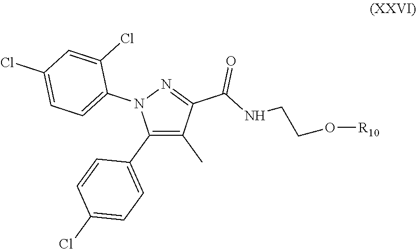

wherein R₁₀ is as defined herein.

In some embodiments, R₁₀ is selected from —H, —C₁-C₂₅alkyl, —C₂-C₂₅alkenyl, —C₂-C₂₅alkynyl, —C₆-C₁₀aryl, each of which being optionally substituted by at least one functionality selected from an hydroxyl, an amine, a halide, —C₁-C₅alkyl, —C₂-C₅alkenyl, —C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-aryl-Cl, idebenonyl-derivative, -pyridine-3-C(═O)—OH and —NR′R″R′″;
each of R′, R″ and R′″ is independently selected from —H, C₁-C₅alkyl, C₂-C₅alkenyl, C₂-C₅alkynyl, —C(═O)—C₂-C₂₅alkyl, —C(═O)—C₂-C₂₅alkenyl and C₅-C₂₅alkynyl; or wherein one of R′, R″ and R′″ is absent.

In some embodiments, the compound is of the general formula (XXVII):

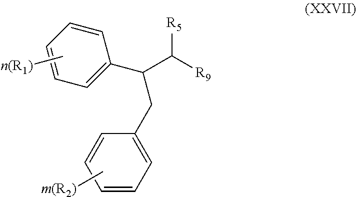

wherein each of R₁, R₂, n, m is as defined herein; R₅ is absent or selected from H, —C₁-C₃alkyl, —C(═O)—O—R₈, —C(═O)—NR′—R₈, halide, CN, and OH; and R₉ is selected from —C(═O)—O—R₈, —C(═O)—NR′—R₈, —NH—C(═O)—O—R₈, —NH—C(═O)—NR′—R₈, —O—C(═O)—O—R₈ and —O—C(═O)—NR′—R₈; R₈ is as defined herein.

In some embodiments, R₅ is a —C₁-C₃ alkyl and R₉ is selected from —C(═O)—C₁-C₂₅alkyl, —C(═O)—O—R₈, —C(═O)—NR′—R₈, —NH—C(═O)—O—R₈, —NH—C(═O)—NR′—R₈, —O—C(═O)—O—R₈ and —O—C(═O)—NR′—R₈; R₈ is as defined herein.

In some embodiments, R₉ is —NH—C(═O)—O—R₈, —NH—C(═O)—NR′—R₈, —O—C(═O)—O—R₈ or —O—C(═O)—NR′—R₈; R₈ is as defined herein.

In some embodiments, R₉ is —NH—C(═O)—O—R₈ or —O—C(═O)—O—R₈; R₈ is as defined herein.

In some embodiments, the compound is of the general formula (XXVIII)

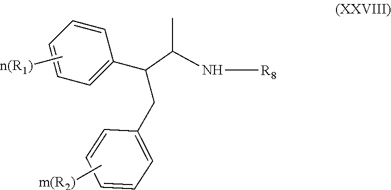

wherein each of R₁, R₂, n, m and R₈ is as defined herein.

In some embodiments, R₈ is —C₁-C₂₅alkyl.

In some embodiments, R₈ is —C₂-C₂₅alkenyl.

In some embodiments, R₈ is —C₂-C₂₅alkynyl.

In some embodiments, R₈ is —C₆-C₁₀aryl.

In some embodiments, R₈ is C₃-C₁₀heteroaryl.

In some embodiments, R₈ is —C₁-C₂₅alkyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

In some embodiments, R₈ is —C₂-C₂₅alkenyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

In some embodiments, R₈ is —C₂-C₂₅alkynyl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

In some embodiments, R₈ is —C₆-C₁₀aryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

In some embodiments, R₈ is C₃-C₁₀heteroaryl substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

In some embodiments, each of n and m is 1.

In some embodiments, R₁ is CN and R₂ is a halide.

In some embodiments, R₂ is a chloride atom.

In some embodiments, the compound is of the formula (XXIX):

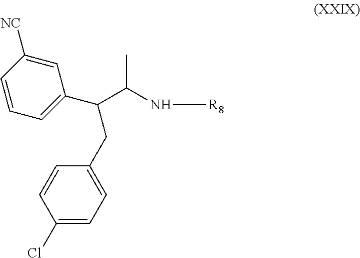

wherein R₈ is as defined herein.

In some embodiments, in a compound of formula (I) each of R₁ and R₂, independently of the other is a group selected from H, a halide and —CN;
each of n and m, independently of the other, is an integer between 0 and 5, designating the number of substituents on the ring;
X is CH, CH₂ or wherein the group C—R₄ is C═R₄;
R₃ is H or a carbon containing group having between 1 and 3 carbon atoms, further optionally substituted;
R₄ is a nitrogen atom or a nitrogen containing group, or a carbon containing group having between 1 and 3 carbon atoms, further optionally substituted;
or R₃ and R₄ together with the atoms to which they are bonded (carbon atom and X, respectively) form a 5- or 6-membered carbocyclic ring optionally containing between 1 and 3 heteroatoms selected from N, O and S.

In some embodiments, X—R₄ is C═R₄ and R₄ is a nitrogen atom.

In some embodiments, R₃ is a carbon containing group and R₄ is a nitrogen containing group.

In some embodiments, R₃ and R₄ together with the atoms to which they are bonded form a 5-membered carbocyclic ring optionally containing 1 or 2 nitrogen atoms.

In some embodiments, the compound is of the general formula (XXX):

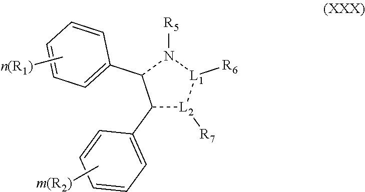

wherein
one of L₁ and L₂ is a nitrogen atom and the other of L₁ and L₂ is a carbon atom (being selected from C, CH or CH₂);
each of R₅, R₆ and R₇, independently of the other, may be absent or selected from —H, C₁-C₃alkyl, —C(═O)—O—R₈, —C(═O)—NR′—R₈, halide, CN, OH, and NR′R″;
and wherein R₈, R′, R″ and R′″ is as defined above. and wherein each bond between C—N, N-L₁, L₁-L₂ and L₂-C (designated ---) is a single or double bond.

In some embodiments, L₁ is nitrogen atom and L₂ is a carbon atom.

In some embodiments, L₁ is a nitrogen and L₂ is a carbon atom, the bond between C and N is a double bond, the bond between N and L₁ is a single bond, and the bond between L₁ and L₂ is a single bond.

In some embodiments, the compound is of formula (XXXI):

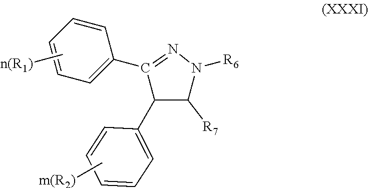

wherein each of R₁, R₂, n, m, R₆ and R₇ are as defined herein.

In some embodiments, R₇ is H and R₆ is selected from —C₁-C₃alkyl, —C(═O)—O—R₈, —C(═O)—NR′—R₈, a halide, —CN, —OH, and —NR′R″; and wherein R₈ is as defined above.

In some embodiments, R₆ is a substituted —C₁-C₃alkyl and R₇ is H.

In some embodiments, the compound is of the formula (XXXII):

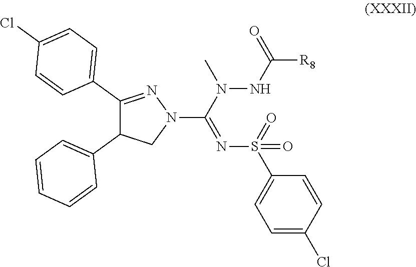

wherein R₈ is as defined herein.

In some embodiments, the compound is of the formula (XXXIII):

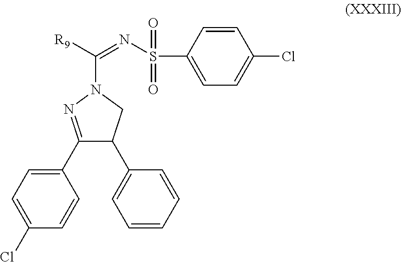

wherein R₉ is selected from —O—R₈ and —NR′—R₈; R₈ is as defined herein.

In some embodiments, the compound is of the formula (XXXIV):

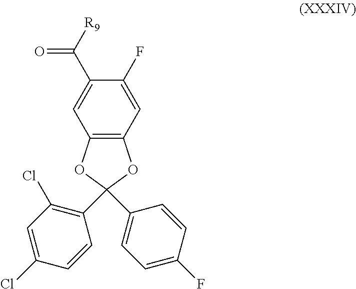

wherein R₉ is selected from —O—R₈ and —NR′—R₈; wherein each of R′ and R₈ is as defined herein.

In some embodiments, the compound is of the formula (XXXV):

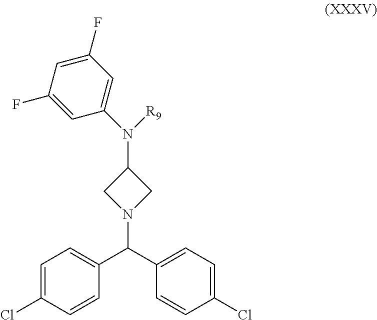

wherein R₉ is selected from —O—RB and —NR′—R₈; wherein each of R′ and R₈ is as defined herein.

In some embodiments, the compound is of the formula (XXXVI):

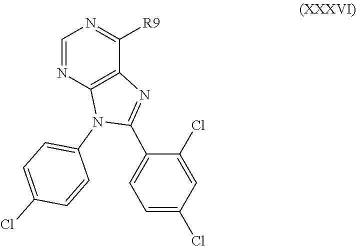

wherein R₉ is selected from —O—RB and —NR′—R₈; wherein each of R′ and R₈ is as defined herein.

As stated herein, the invention provides a compound that is of the general formula (II), as defined herein. In other words, the compound is of the formula:

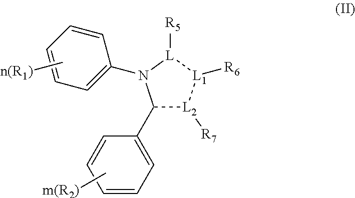

wherein
one of L, L₁ and L₂ is a nitrogen atom and the others of L, L₁ and L₂ are each a carbon atom (being selected from C, CH or CH₂);
each of R₅, R₆ and R₇, independently of the other, may be selected from —H, —C₁-C₃alkyl, —C(═O)—OH, —C(═O)—O—R₈, —C(═O)—NR′R₈, halide, —CN, —OH, and —NR′R″; or
one of R₅ and R₆ or R₆ and R₇ together with the atoms to which they bond may form a 5-, 6-, 7- or 8-membered carbocyclic ring optionally containing between 1 and 3 heteroatoms selected from N, O and S;
the 5-, 6-, 7- or 8-membered carbocyclic ring is further optionally substituted by at least one functionality B selected from —H, —C₁-C₂₅alkyl, —C₂-C₂₅alkenyl, —C₂-C₂₅alkynyl, —C₆-C₁₀aryl, an hydroxyl, an amine, a halide, —ONO₂, —NO₂, —S—, —S—C₁-C₅alkyl, —S—C₂-C₅alkenyl, —S—C₂-C₅alkynyl, —C(═O)—, —C(═O)—C₁-C₂₅alkyl, —C(═O)—O—C₁-C₅alkyl, —C(═O)—O—C₂-C₅alkenyl, —C(═O)—O—C₂-C₅alkynyl, —C(═O)—NR′R″R′″, —C(═O)—NR′—C(═O)—C₁-C₂₅alkyl, —C(═O)—NR′—C(═O)—C₂-C₂₅alkenyl, —C(═O)—NR′—C(═O)—C₂-C₂₅alkynyl, —C(═O)—OR₁₀, —O—C₁-C₅alkyl, —O—C₁-C₅alkenyl, —O—C₁-C₅alkynyl, —NH—NH₂, NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″;
the 5-, 6-, 7- or 8-membered carbocyclic ring may be optionally substituted by at least one functionality B selected from structures (A) through (H):

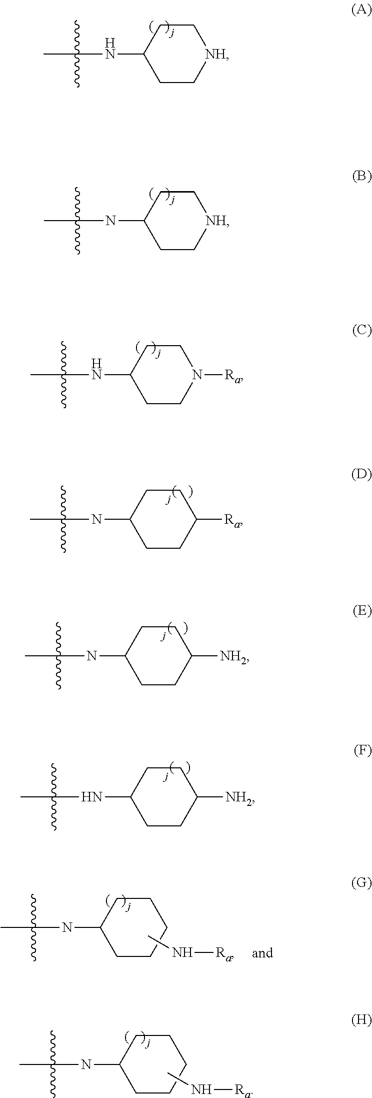

wherein in each functionality (A) through (H), the wavy line indicates point or bond of connectivity, j is 0 or 1 and Ra is selected from —H, —C₁-C₂₅alkyl, —C₂-C₂₅alkenyl, —C₂-C₂₅alkynyl, —C(═O)—C₆-C₁₀aryl and —C(═O)—C₃-C₁₀heteroaryl,
wherein in functionalities (G) and (H) the pendant —NH—Ra group may appear between 1 and 11 times at any position along the carbocycle (in some embodiments, it may be positioned at a ring atom once removed, twice removed or three times removed from the existing group or endocyclic N atom; in some embodiments, the position of the functionality is 1, 2 or 1, 3 or 1,4, wherein 1 designates the position of the existing group or the endocyclic N atom);
one of R₅, R₆ and R₇ may be absent;
R₈ is selected from —H, —C₁-C₂₅alkyl, —C₂-C₂₅alkenyl, —C₂-C₂₅alkynyl, —C₆-C₁₀aryl and C₃-C₁₀heteroaryl, each of which being optionally substituted by at least one functionality selected from an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″;
R₁₀ is selected from —H, —C₁-C₂₅alkyl, —C₂-C₂₅alkenyl, —C₂-C₂₅alkynyl, —C₆-C₁₀aryl, each of which being optionally substituted by at least one functionality selected from an hydroxyl, an amine, a halide, —C₁-C₅alkyl, —C₂-C₅alkenyl, —C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-aryl-Cl, idebenonyl-derivative, -pyridine-3-C(═O)—OH and —NR′R″R′″;
each of R′, R″ and R′″ is independently selected from —H, C₁-C₅alkyl, C₂-C₅alkenyl, C₂-C₅alkynyl, —C(═O)—C₂-C₂₅alkyl, —C(═O)—C₂-C₂₅alkenyl and C₅-C₂₅alkynyl;
or wherein one of R′, R″ and R′″ is absent; and wherein each bond between N-L, L-L₁, L₁-L₂ and L₂-C (designated ---) is a single or double bond.

In some embodiments, in a compound of formula (II), L₂ is a nitrogen atom and each of L and L is a carbon atom. In some embodiments, R₇ is absent and R₅ and R₆ together with the atoms to which they bond form a 5-, 6-, 7- or 8-membered carbocyclic ring optionally containing between 1 and 3 heteroatoms selected from N, O and S. In some embodiments, the compound is of the formula (XXXVII):

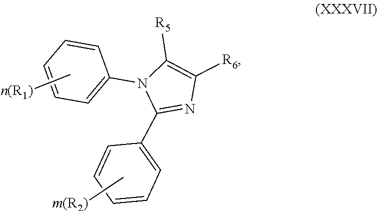

wherein each of R₁, R₂, R₅, R₆, n and m is as defined above.

In some embodiments, R₅ and R₆ together with the atoms to which they bond may form a 5-, 6-, 7- or 8-membered carbocyclic ring optionally containing between 1 and 3 heteroatoms selected from N, O and S.

In some embodiments, the compound is of formula (XXXVIII):

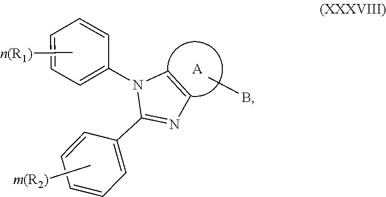

wherein each of R₁, R₂ and m is as defined above, ring A is a 5-, 6-, 7- or 8-membered carbocyclic ring optionally containing between 1 and 3 heteroatoms selected from N, O and S, and further optionally substituted by a group B selected from —H, —C₁-C₂₅alkyl, —C₂-C₂₅alkynyl, —C₆-C₁₀aryl, an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₂-C₅alkenyl, —C(═O)—O—C₂-C₅alkynyl, —C(═O)—NR′R″R′″, —C(═O)—NR′—C(═O)—C₁-C₂₅alkyl, —C(═O)—NR′—C(═O)—C₂-C₂₅alkenyl, —C(═O)—NR′—C(═O)—C₂-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″;
the 5-, 6-, 7- or 8-membered carbocyclic ring may be optionally substituted by at least one functionality B selected from structures (A) through (H):

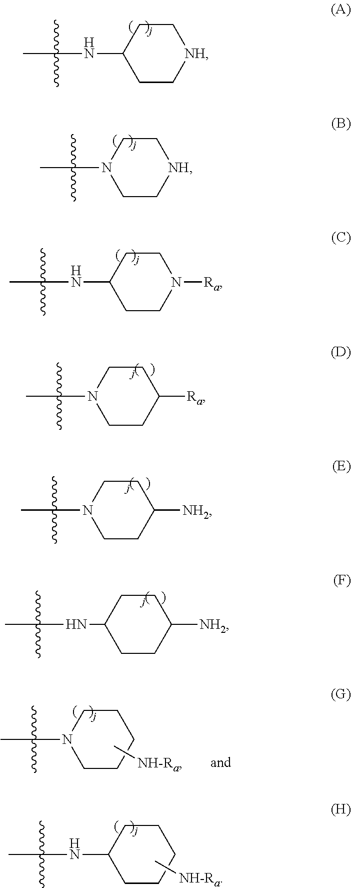

wherein in each functionality (A) through (H), the wavy line indicates point or bond of connectivity, j is 0 or 1 and Ra is selected from —H, —C₁-C₂₅alkyl, —C₂-C₂₅alkenyl, —C₂-C₂₅alkynyl, —C(═O)—C₆-C₁₀aryl and —C(═O)—C₃-C₁₀heteroaryl,
wherein in functionalities (G) and (H) the pendant —NH—Ra group may appear between 1 and 11 times at any position along the carbocycle (in some embodiments, it may be positioned at a ring atom once removed, twice removed or three times removed from the existing group or endocyclic N atom; in some embodiments, the position of the functionality is 1, 2 or 1, 3 or 1,4, wherein 1 designates the position of the existing group or the endocyclic N atom).

In some embodiments, ring A is a 5-membered ring. In some embodiments, the ring is a heterocyclic ring comprising one or more heteroatom selected from N, O and S.

In some embodiments, ring A is a 6-membered ring. In some embodiments, the ring is a heterocyclic ring comprising one or more heteroatom selected from N, O and S. In some embodiments, the ring is an aromatic ring or a heteroaryl ring.

In some embodiments, ring A is a 7-membered ring. In some embodiments, the ring is a heterocyclic ring comprising one or more heteroatom selected from N, O and S.

In some embodiments, ring A comprises one or more double bonds.

In some embodiments, the compound is a compound of the formula (XXXIX):

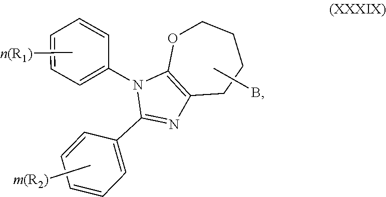

wherein each of R₁, R₂, n, m and B is as defined above.

In some embodiments, the compound is a compound of formula (XXXX):

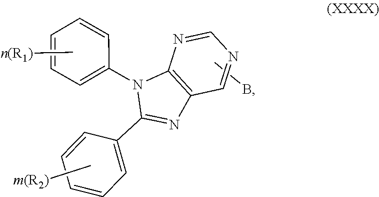

wherein each of R₁, R₂, n, m and B is as defined above.

In some embodiments, the compound if a compound of formula (XXXXI):

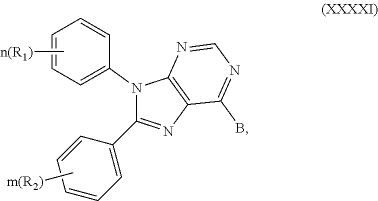

wherein each of R₁, R₂, n, m and B is as defined above.

In some embodiments, the compound of formula (II) is a compound having the structure of formula (XXXXII):

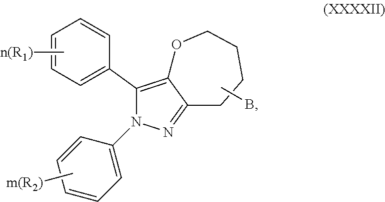

wherein each of R₁, R₂, m and B is as defined above.

In some embodiments of compounds of the invention, n is 2 and m is 1, or m is 2 and n is 1, or each of m and n is either 2 or 1. In some embodiments, R₁ and R₂ are each a halide. In some embodiments, each of R₁ and R₂ is a chloride atom.

In some embodiments of compounds of the invention, n and m together represent 2 or 3 halide atoms. In some embodiments, the halide atoms are each a chloride atom.

In some embodiments, the following compounds of the invention are provided:

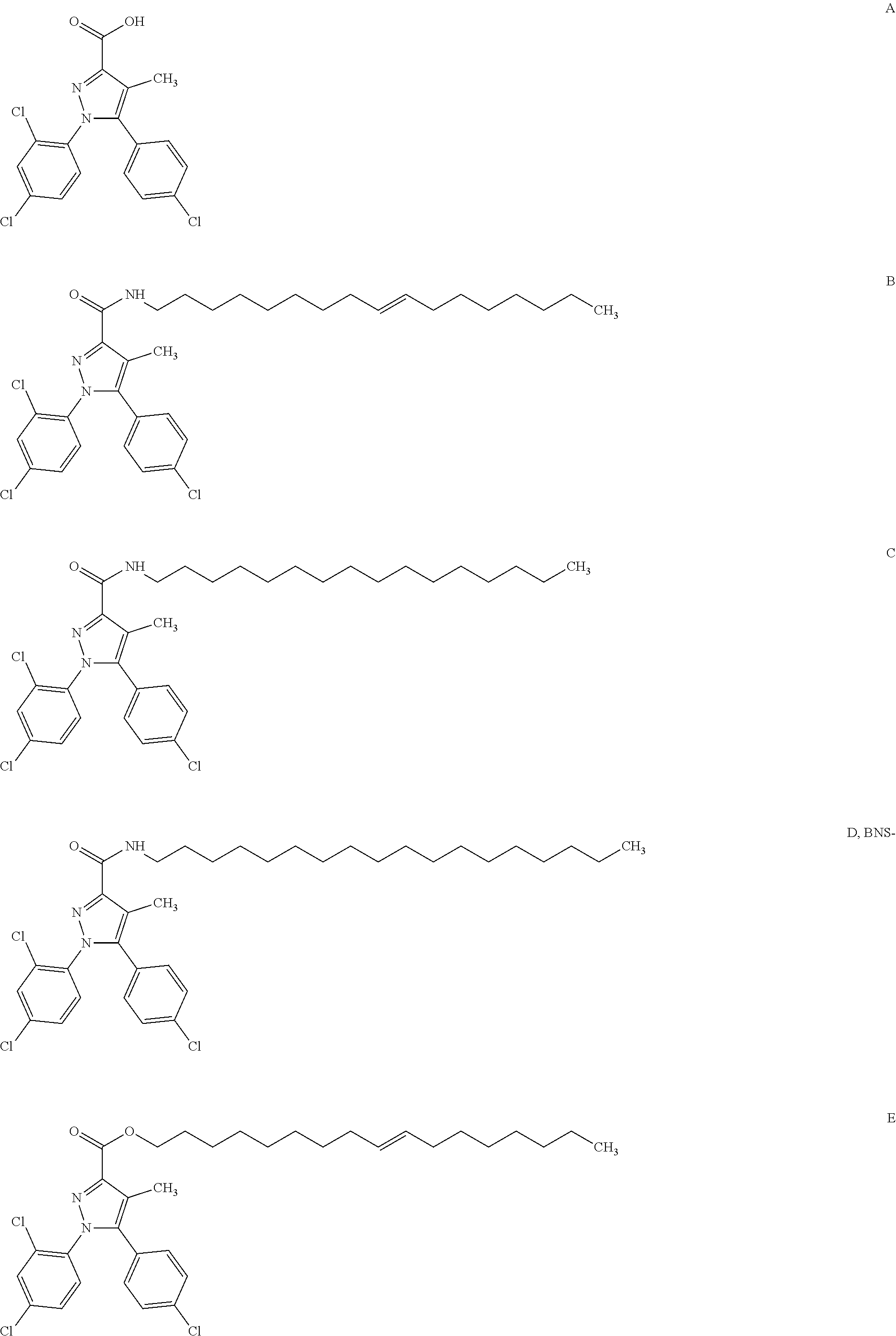 [SMILES: Cc1c(C(=O)O)nn(-c2ccc(Cl)cc2Cl)c1-c1ccc(Cl)cc1 ; CCCCCCC/C=C/CCCCCCCCNC(=O)c1nn(-c2ccc(Cl)cc2Cl)c(-c2ccc(Cl)cc2)c1C ; CCCCCCCCCCCCCCCCNC(=O)c1nn(-c2ccc(Cl)cc2Cl)c(-c2ccc(Cl)cc2)c1C ; CCCCCCCCCCCCCCCCCCNC(=O)c1nn(-c2ccc(Cl)cc2Cl)c(-c2ccc(Cl)cc2)c1C ; CCCCCCC/C=C/CCCCCCCCOC(=O)c1nn(-c2ccc(Cl)cc2Cl)c(-c2ccc(Cl)cc2)c1C]

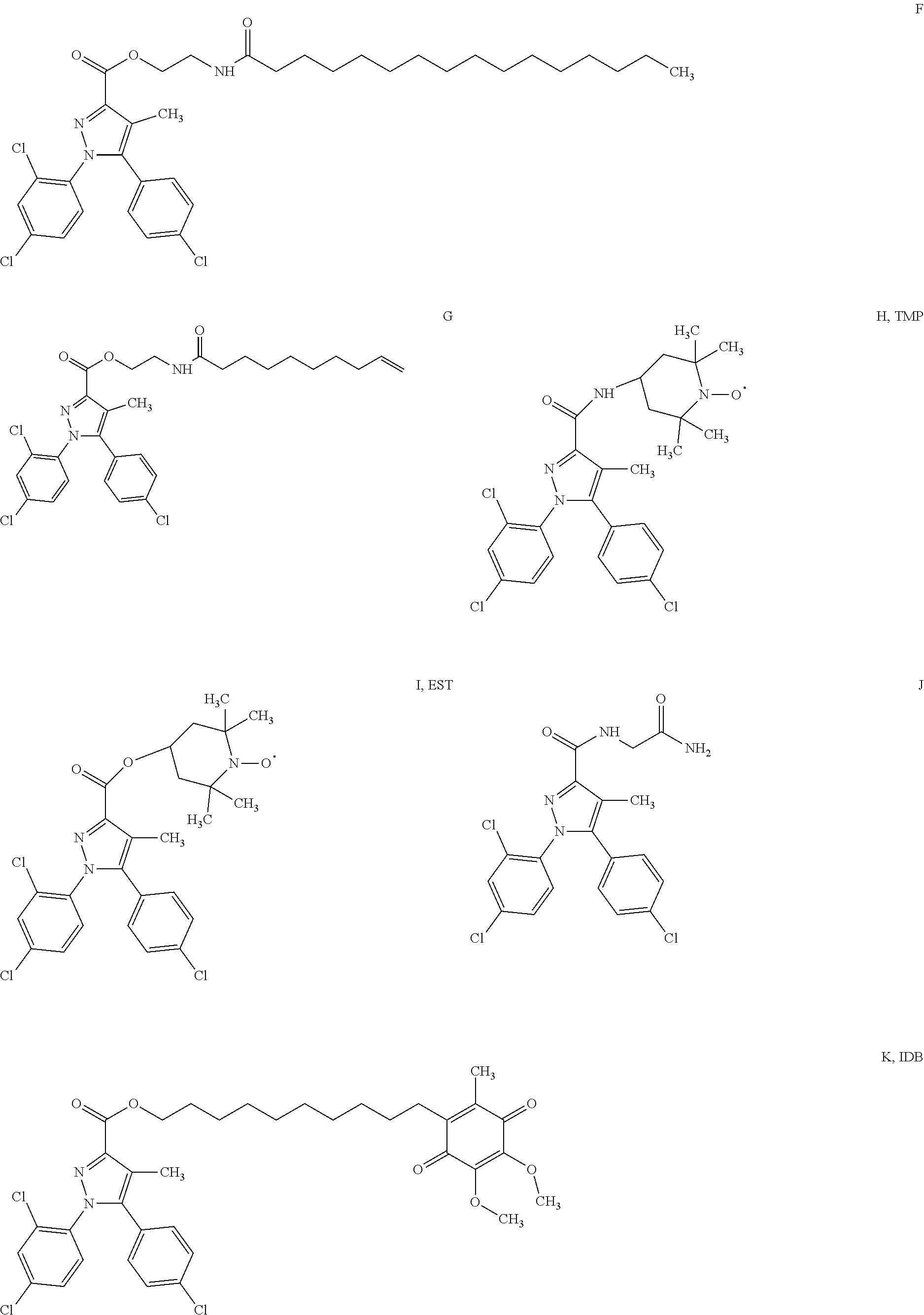

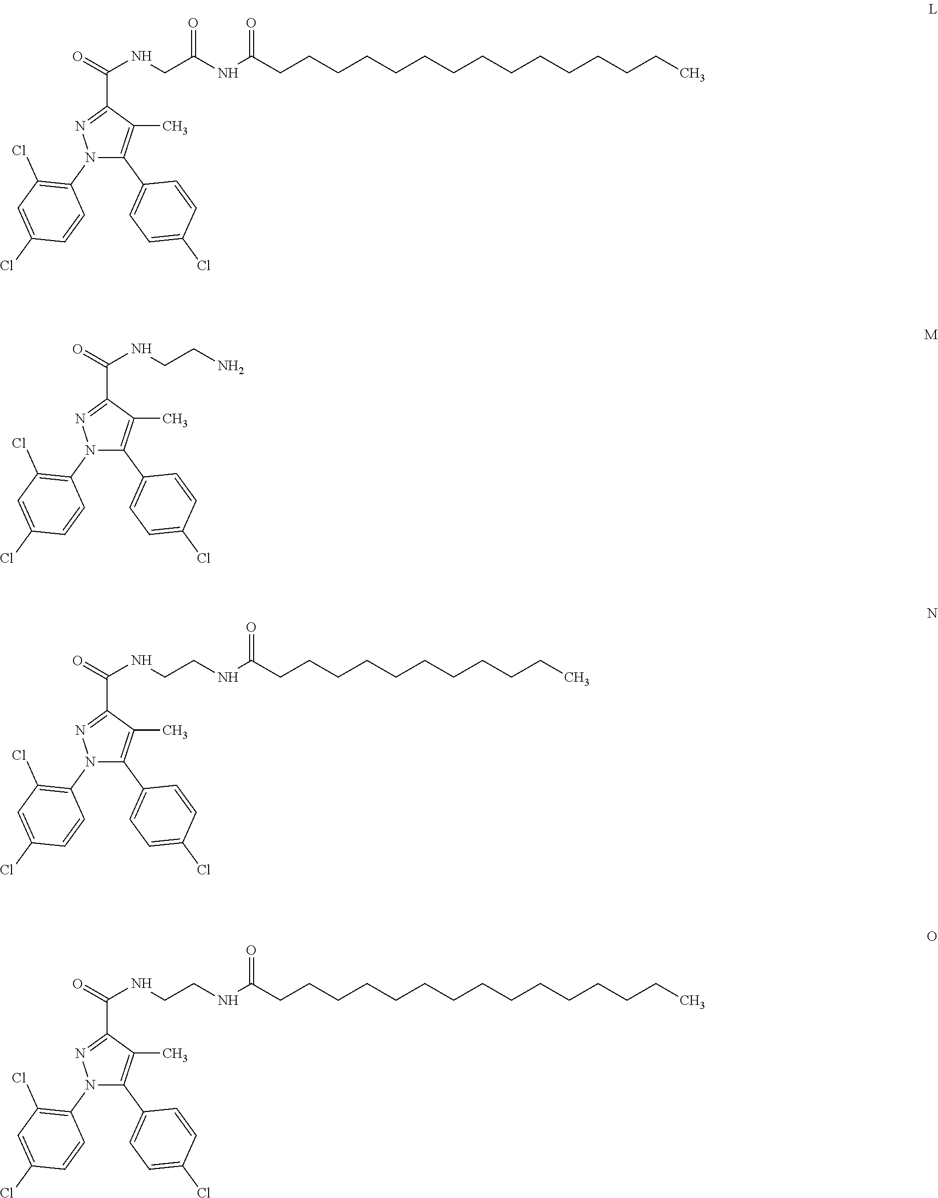 [SMILES: CCCCCCCCCCCCCCCC(=O)NC(=O)CNC(=O)c1nn(-c2ccc(Cl)cc2Cl)c(-c2ccc(Cl)cc2)c1C ; Cc1c(C(=O)NCCN)nn(-c2ccc(Cl)cc2Cl)c1-c1ccc(Cl)cc1 ; CCCCCCCCCCCC(=O)NCCNC(=O)c1nn(-c2ccc(Cl)cc2Cl)c(-c2ccc(Cl)cc2)c1C ; CCCCCCCCCCCCCCCC(=O)NCCNC(=O)c1nn(-c2ccc(Cl)cc2Cl)c(-c2ccc(Cl)cc2)c1C]

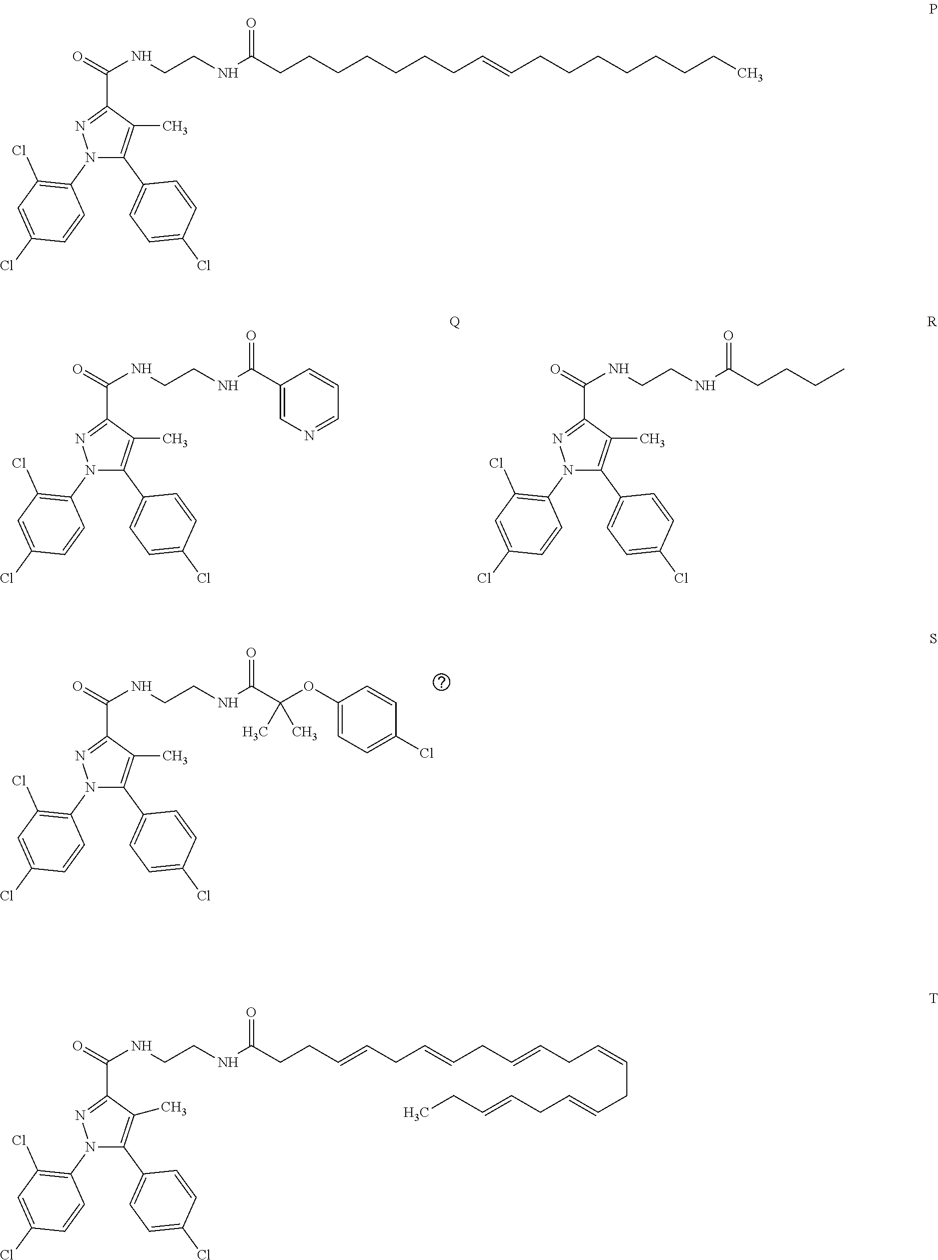 [SMILES: CCCCCCCC/C=C/CCCCCCCC(=O)NCCNC(=O)c1nn(-c2ccc(Cl)cc2Cl)c(-c2ccc(Cl)cc2)c1C ; Cc1c(C(=O)NCCNC(=O)c2cccnc2)nn(-c2ccc(Cl)cc2Cl)c1-c1ccc(Cl)cc1 ; CCCCC(=O)NCCNC(=O)c1nn(-c2ccc(Cl)cc2Cl)c(-c2ccc(Cl)cc2)c1C ; Cc1c(C(=O)NCCNC(=O)C(C)(C)Oc2ccc(Cl)cc2)nn(-c2ccc(Cl)cc2Cl)c1-c1ccc(Cl)cc1 ; CC/C=C/C/C=C/C/C=C\C/C=C/C/C=C/C/C=C/CCC(=O)NCCNC(=O)c1nn(-c2ccc(Cl)cc2Cl)c(-c2ccc(Cl)cc2)c1C]

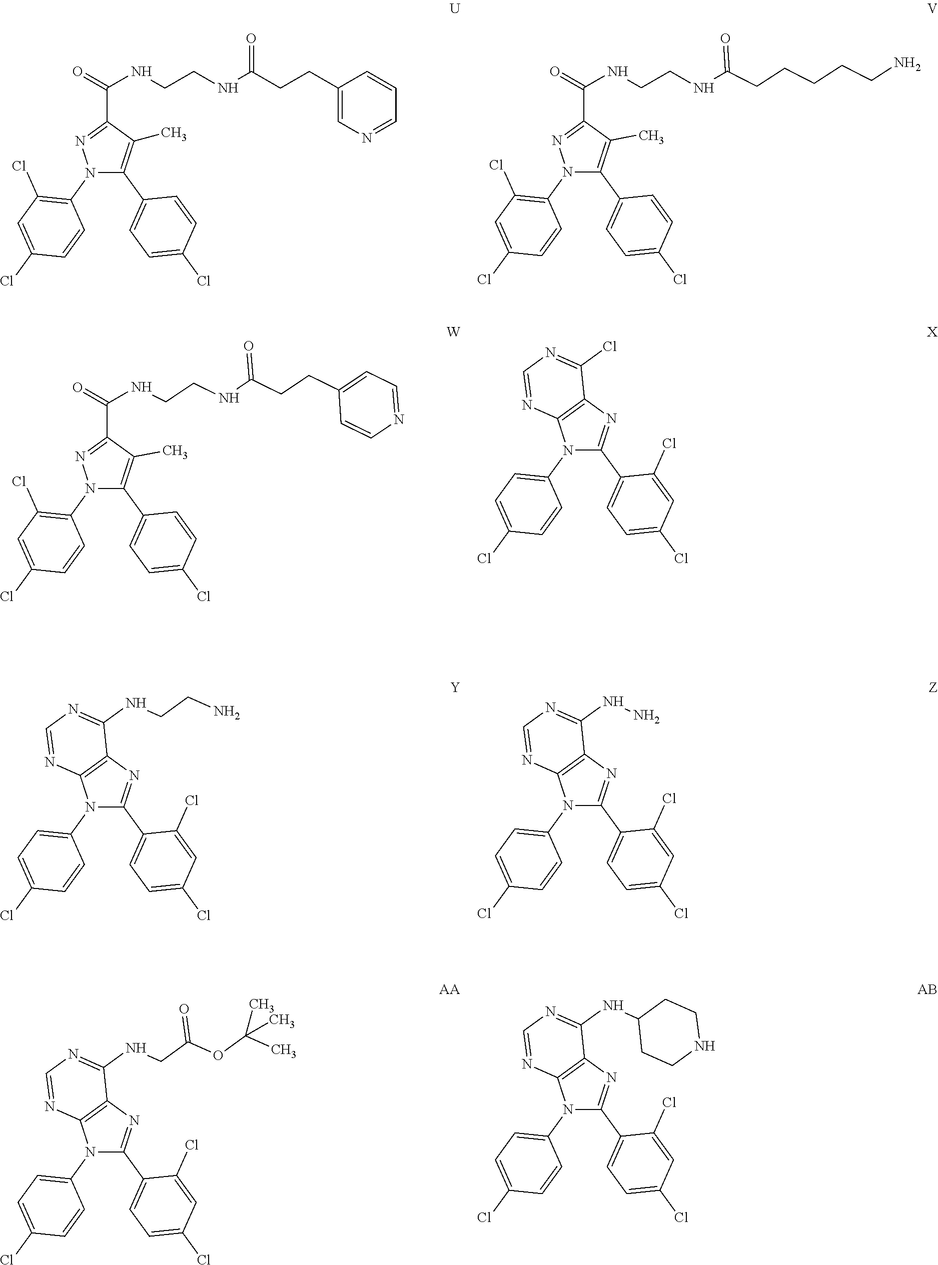

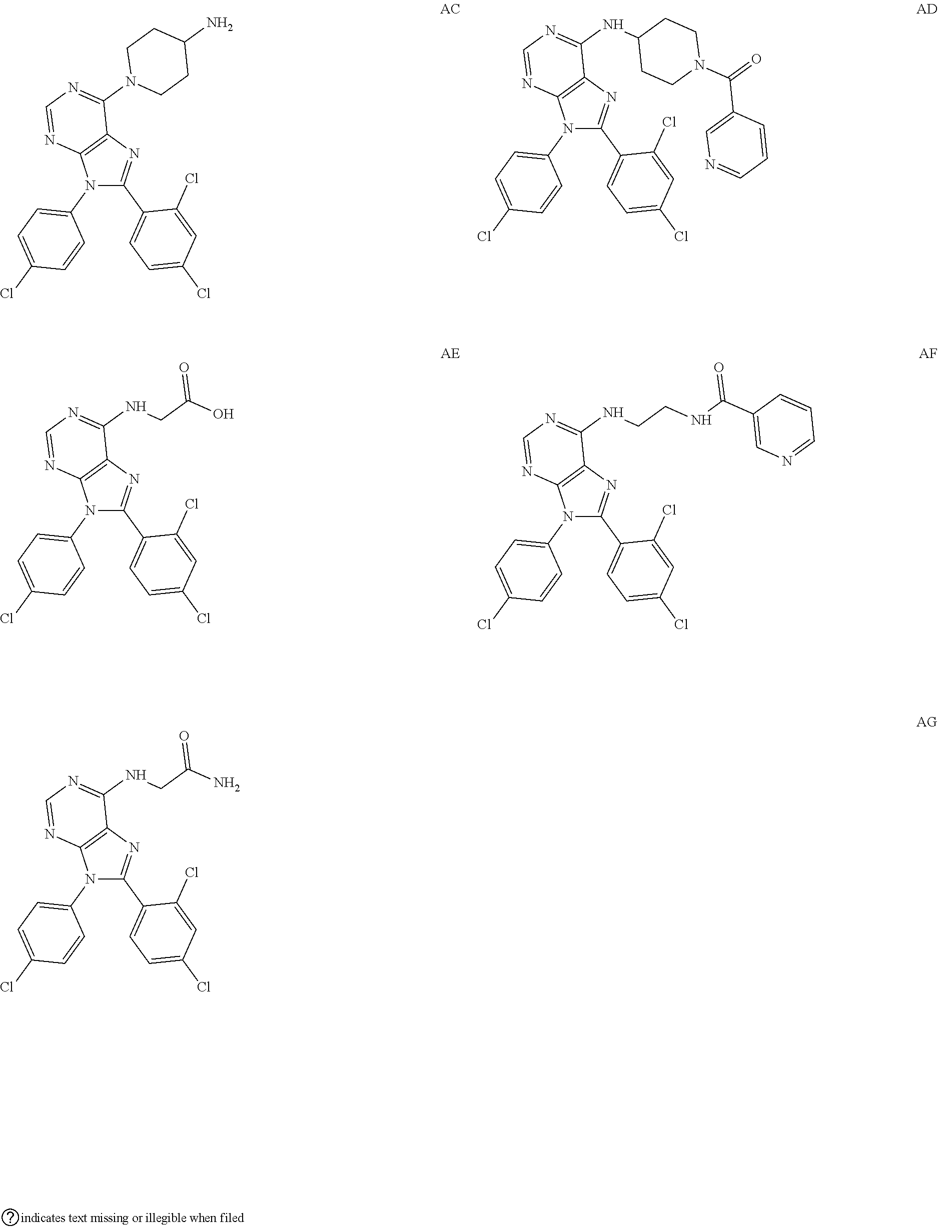 [SMILES: NC1CCN(c2ncnc3c2nc(-c2ccc(Cl)cc2Cl)n3-c2ccc(Cl)cc2)CC1 ; O=C(c1cccnc1)N1CCC(Nc2ncnc3c2nc(-c2ccc(Cl)cc2Cl)n3-c2ccc(Cl)cc2)CC1 ; O=C(O)CNc1ncnc2c1nc(-c1ccc(Cl)cc1Cl)n2-c1ccc(Cl)cc1 ; O=C(NCCNc1ncnc2c1nc(-c1ccc(Cl)cc1Cl)n2-c1ccc(Cl)cc1)c1cccnc1 ; NC(=O)CNc1ncnc2c1nc(-c1ccc(Cl)cc1Cl)n2-c1ccc(Cl)cc1]

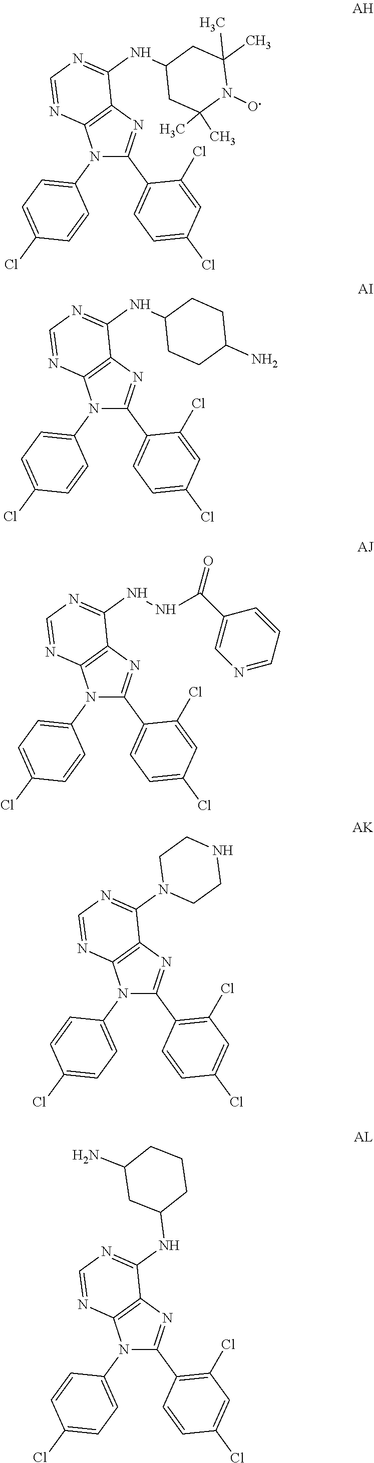

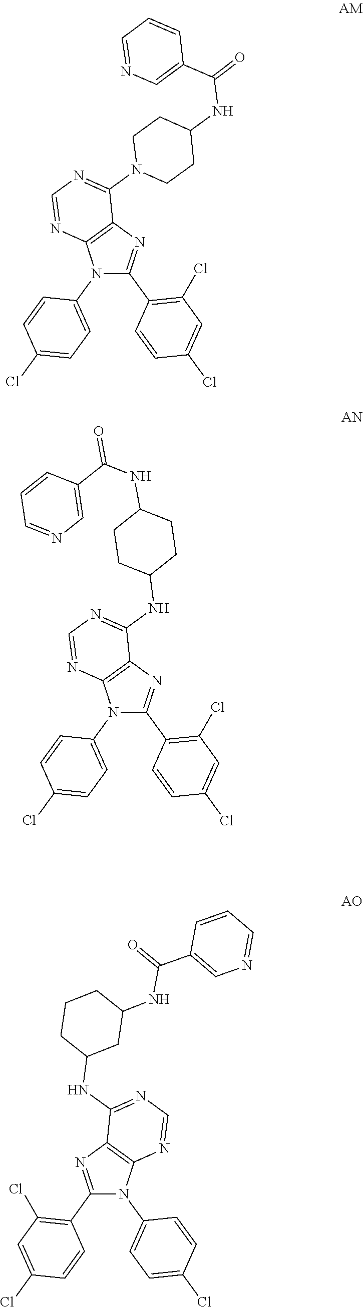 [SMILES: O=C(NC1CCN(c2ncnc3c2nc(-c2ccc(Cl)cc2Cl)n3-c2ccc(Cl)cc2)CC1)c1cccnc1 ; O=C(NC1CCC(Nc2ncnc3c2nc(-c2ccc(Cl)cc2Cl)n3-c2ccc(Cl)cc2)CC1)c1cccnc1 ; O=C(NC1CCCC(Nc2ncnc3c2nc(-c2ccc(Cl)cc2Cl)n3-c2ccc(Cl)cc2)C1)c1cccnc1]

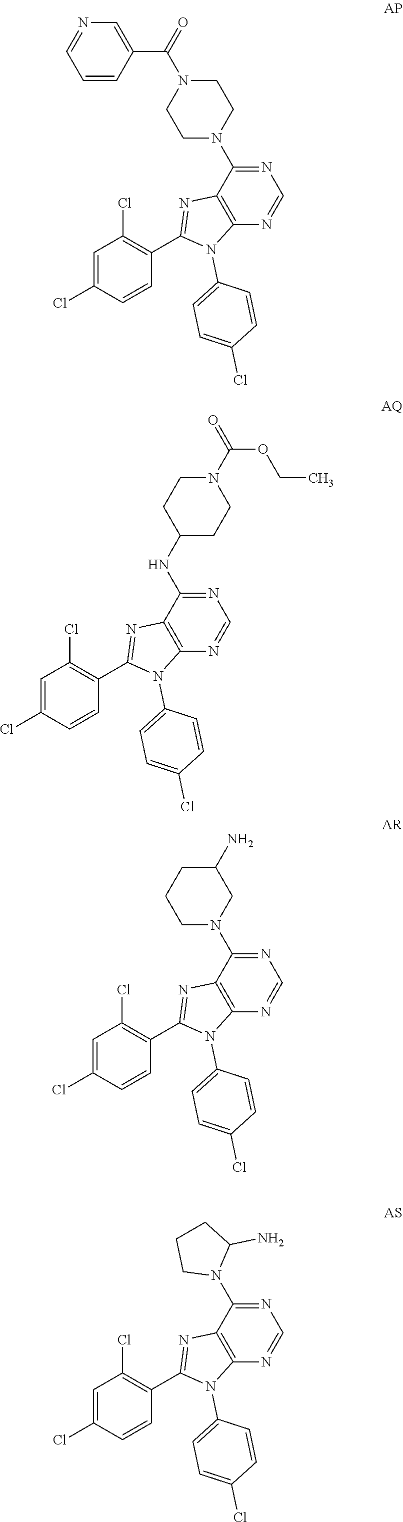 [SMILES: O=C(c1cccnc1)N1CCN(c2ncnc3c2nc(-c2ccc(Cl)cc2Cl)n3-c2ccc(Cl)cc2)CC1 ; CCOC(=O)N1CCC(Nc2ncnc3c2nc(-c2ccc(Cl)cc2Cl)n3-c2ccc(Cl)cc2)CC1 ; NC1CCCN(c2ncnc3c2nc(-c2ccc(Cl)cc2Cl)n3-c2ccc(Cl)cc2)C1 ; NC1CCCN1c1ncnc2c1nc(-c1ccc(Cl)cc1Cl)n2-c1ccc(Cl)cc1]

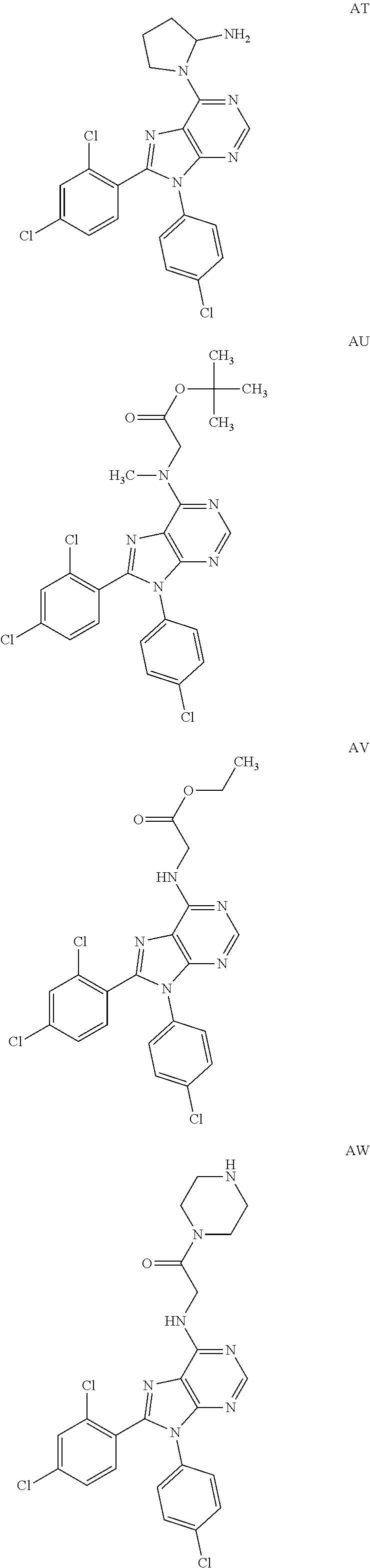

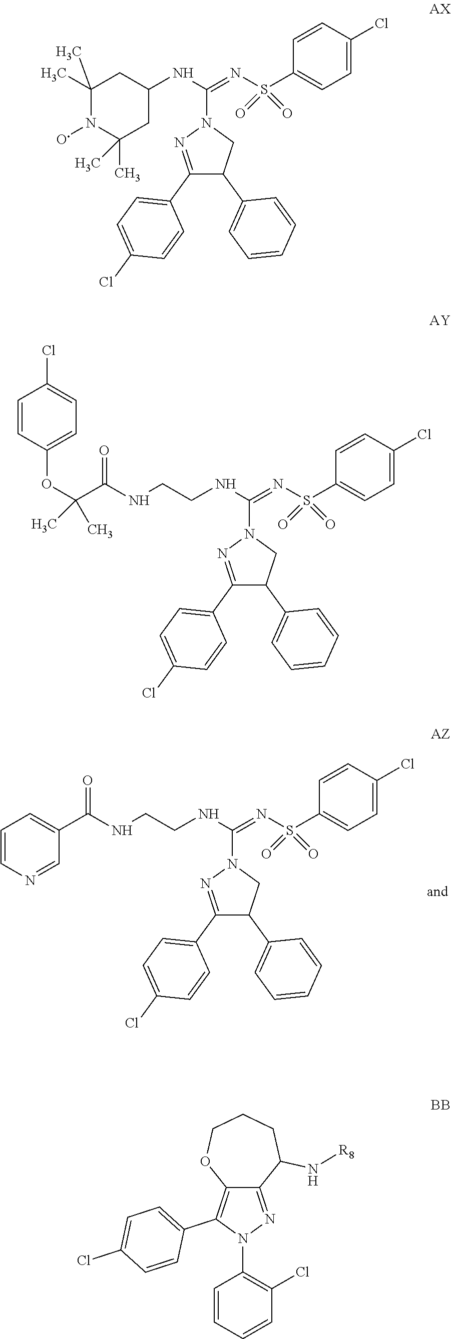

In some embodiments, in each compound of the invention, R₈ is a lipophilic moiety.

Compounds of the invention may be used as modulators of peripheral cannabinoid receptors, including peripherally restricted CB1 receptors and CB2 receptors. In some embodiments, the compounds are modulators (e.g., inhibiting) of a peripherally restricted CB1 receptor. In some embodiments, the compounds are neutral antagonists or inverse agonists. In some embodiments, the compounds are modulators (e.g., activating) of CB2 receptors.

As used herein, the expression “peripherally restricted CB₁ receptor blocker” refers to agents/materials according to the invention that are antagonists or blockers of CB1 receptors present in peripheral organs and tissues, including the adipose tissues, the liver, skeletal muscles, pancreatic β-cells and the kidneys, without causing centrally-mediated side effects. In other words, these blockers or antagonists retain the therapeutic benefits of globally acting CB1 receptor blockers without causing CNS-mediated side effect.

A “CB₁ receptor blocker” or antagonist is a compound according to the invention, which in most general terms partially or fully blocks, inhibits, or neutralizes a biological function of a peripheral CB1 receptor. By partially or fully blocking, inhibiting, or neutralizing a biological function of the receptor, prevention or treatment of a variety of metabolic syndromes can be achieved. These metabolic syndromes include obesity, insulin resistance, diabetes, coronary heart disease, fatty liver, hepatic cirrhosis, chronic kidney disease and cancer.

The invention further provides a compound of formula (I) as a peripherally restricted CB1 receptor inverse agonist.

The invention further provide a composition comprising a compound of the invention. In some embodiments, the composition is a pharmaceutical composition in a form suitable for administration to a human or animal subject. As used herein, the “pharmaceutical composition” comprises a therapeutically effective amount of a compound of the invention, optionally together with suitable additives such as diluents, preservatives, solubilizers, emulsifiers, adjuvant and/or carriers. The compositions may be liquids or lyophilized or otherwise dried formulations and include diluents of various buffer content (e.g.; Tris-HCL, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), and others.

Compositions suitable for oral administration can comprise of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions or self-emulsifying formulations. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers. Tablet forms can include one or more of lactose, sucrose, mannitol, corn starch, potato starch, alginic acid, microcrystalline cellulose, acacia, gelatin, guar gum, colloidal silicon dioxide, croscarmellose sodium talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Compositions suitable for parenteral administration include sterile nanoemulsions, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives.

Compounds of the invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid.

Compounds of the present invention may be made into injectable formulations. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, eds., pages 238-250 (1982), and ASHP Handbook on Injectable Drugs, Toissel, 4th ed., pages 622-630 (1986).

In some embodiments, the composition is suitable for oral administration.

In other embodiments, the composition is suitable for IV (intravenous) or IM (intramuscular) administration.

In some embodiments, the composition is a self-emulsifying oil formulation comprising nanocarriers according to the invention.

In another one of its aspects, the invention provides a nanocarrier comprising at least one compound according to the invention.

The nanocarrier may be a nanoparticle, a nanocapsule or mixtures thereof. A “nanocarrier” of the invention is a particulate material that is biocompatible and sufficiently resistant to chemical and/or physical destruction, such that a sufficient amount of the nanocarriers remain substantially intact after administration into the human or animal body and for sufficient time to be able to reach the desired target tissue (or organ). Generally, the nanocarriers are of average diameters of up to 700 nm.

Depending on various parameters associated with a compound of the invention (e.g. solubility, molecular weight, polarity, electrical charge, reactivity, chemical stability, biological activity, and others), the compound may be contained (encapsulated) in nanocapsules (NCs), and/or embedded in a matrix making-up nanoparticle (NPs). For the chosen application, the nanocarrier may therefore be in the form of core/shell (termed hereinafter also as nanocapsule), having a polymeric shell and a core containing at least one compound of the invention.

Alternatively, the nanoparticles may be of a substantially uniform composition not featuring a distinct core/shell structure. These nanocarriers are herein referred to as nanoparticles (NPs).

In some embodiments, the average diameter of the nanocarrier is between about 100 and 200 nm. In some embodiments, the average diameter is between about 200 and 300 nm. In some embodiments, the average diameter is between about 300 and 400 nm, the average diameters between 400 and 500 nm. In some embodiments, the average diameter is between about 600 and 700 nm.

In some other embodiments, the average diameter of the nanocarrier is between about 50 and 700 nm. In other embodiments, the average diameter is between about 50 and 500 nm. In other embodiments, the average diameter is between about 50 and 400 nm. In further embodiments, the average diameter is between about 50 and 300 nm. In further embodiments, the average diameter is between about 50 and 200 nm. In further embodiments, the average diameter is between about 50 and 100 nm.

Materials suitable for forming nanocarriers, e.g., nanocapsules and/or nanoparticles according to the invention, are polyesters including polylactic acid (PLA), polyglycolic acid (PGA), polyhydroxybutyrate and polycaprolactone), poly(orthoesters), polyanhydrides, polyamino acid, poly(alkyl cyanoacrylates), polyphophazenes, copolymers of (PLA/PGA) and asparate or polyethylene-oxide (PEO).

In some embodiments, the nanocarrier is a nanoparticle, the nanoparticle comprising a first matrix, wherein a compound of the invention is embedded within the matrix. In other embodiments, the nanocarrier is a nanocapsule, the nanocapsule comprising a first shell encapsulating the compound of the invention or a composition comprising the compound.

The nanocarriers may be further enveloped by another encapsulation layer, thereby forming a double-layered protection. Thus, in some embodiments, the nanocarrier is further encapsulated within a second shell layer, which may comprise the same or different material than that of the first shell layer. In some embodiments, the nanocarrier is further embedded within a second matrix, the first and second matrices may be comprised of the same or different materials.

In order to increase the amount of active compound reaching the target tissue or organ, it is sometimes desired to provide a product comprising a plurality of nanocarriers packed in a single encasing. Therefore, in another aspect, there is provided a nano- or a microcapsule comprising a plurality of nanocarriers of the invention.

According to another aspect, there is provided a nano- or microparticle comprising a plurality of nanocarriers of the invention. Such nano- or microparticles may endow long-acting dosage forms when administered parenterally, or may be used as powders for oral, inhalation or pulmonary delivery of compounds of the invention. In some embodiments, the nano- or microparticle, that comprises a plurality of nanocarriers of the invention, may be formed of a hydrophobic polymer.

Compounds of formula (I) as well as formulations or compositions comprising them may also be used in methods of preventing or treating metabolic syndromes. Accordingly, the invention further provides uses of compounds of the invention in methods of therapeutic prevention or treatment of diseases and disorders associated with CB1 receptor activity, e.g., metabolic syndromes, as defined herein.

The invention further provides methods of prevention and treatment of metabolic diseases and disorders that comprise administering to a human or animal subject an amount of a compound of the invention. The compound may be:
a highly lipophilic derivative of cannabinoid having a Log P (partition coefficient between n-octanol and water) value ranging from 3 and 17;
a compound of the general formula (I) and any derived formula, as defined herein.

As noted hereinabove, the metabolic diseases or disorders or syndromes may be selected from obesity, insulin resistance, diabetes, coronary heart disease, liver cirrhosis and cancer.

Thus, the invention provides a method of treating a subject to reduce body fat, or to reduce body weight, or to treat insulin resistance, or to treat diabetes, or to reduce or control high blood pressure, or to improve a poor lipid profile with elevated LDL cholesterol, low HDL cholesterol, and elevated triglycerides, or to treat fatty liver disease, or to ameliorate chronic kidney disease, or to treat a metabolic syndrome as herein defined, the method comprising administering to the subject a compound of the invention. The compound may be in a form suitable for oral, parenteral, subcutaneous, intravenous, intramuscular or interperitoneal administration.

### BRIEF DESCRIPTION OF SEVERAL VIEWS OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-C depict the results of radioligand displacement assays. BNS-002 is more lipid soluble than rimonabant (estimated partition coefficient [log P], 17 vs. 6.4 for rimonabant) but retains high affinity and selectivity for CB1 receptor. In radioligand displacement assays, BNS-002 has a Ki of 4.96 nM for CB1 receptor, which is similar to that of rimonabant (FIG. 1A). Like rimonabant, BNS-002 reduces GTPγS binding in mouse brain membranes (FIG. 1B) and is able to ameliorate the action of the potent CB1 receptor agonist HU-210 (FIG. 1C), suggesting that it is an inverse agonist.

FIGS. 2A-B demonstrate reduced brain penetrance of BSN002. BSN002 displays markedly reduced brain penetrance, as reflected by its reduced brain levels and increased serum levels following an administration of the compound in two different doses (3 and 10 mg/kg, ip).

FIGS. 3A-E provide comparison of the effects of BNS002 and rimonabant on ambulation. Whether the reduced brain penetrance of BNS-002 is associated with an attenuation of behavioral effects was tested. To that end, the effects of BNS-002 and rimonabant were evaluated in antagonizing cannabinoid-induced hypomotility. The marked increase in immobility induced in mice by the cannabinoid agonist HU-210 (30 μg/kg, ip) was completely blocked by rimonabant (10 mg/kg, ip) but was unaffected by a similar dose and even higher doses of BNS-002 (10, 20, and 50 mg/kg; FIGS. 3A-E).

FIGS. 4A-D show the increased activity profile of rimonabant as compared with BNS002. Rimonabant (10 mg/kg, ip), but not BNS-002 (at 10, 20 and 50 mg/kg, ip), induced a marked increase in the activity profile in mice (FIGS. 4A-D).

FIGS. 5A-B show the metabolic profile of BNS002 and rimonabant. The metabolic profile of BNS-002 and rimonabant was examined in mice with diet-induced obesity (DIO). Male C57BL/6 mice fed a high-fat diet (HFD) for 14 weeks became obese and were then started on daily ip injections of vehicle, rimonabant, or BNS002 (both at 10 mg/kg/d) for an additional 28 days. Age- and sex-matched mice on standard chow served as controls. The overweight and increased adiposity of mice on HFD were significantly reduced by rimonabant only (FIGS. 5A-B).

FIGS. 6A-C show that both rimonanbant and BNS002 upregulate HFD-induced reduction in VO₂, total energy expenditure, and fat oxidation, as measured by using an indirect calorimetry assessment.

FIGS. 7A-B demonstrate the efficacy of rimonabant over BNS002 in reducing food intake. The greater efficacy of rimonabant over BNS-002 in reducing body weight is probably related to its ability to reduce total caloric intake (FIGS. 7A-B).

FIGS. 8A-C show the efficacy of rimonabant and BNS-002 in ameliorating HFD-induced hyperglycemia and glucose tolerance. HFD-induced hyperglycemia and glucose intolerance were completely reversed by BNS-002 in a similar fashion as rimonabant (FIGS. 8A-B). A trend toward reduction in serum insulin levels was also documented by both compounds (FIG. 8C).

FIG. 9 shows the efficacy of rimonabant and BNS-002 in reversing HFD-induced hepatic steatosis. HFD-induced hepatic steatosis, as reflected in elevated fat vacuoles in the liver, was completely reversed by rimonabant and partially by BNS-002.

FIG. 10 shows efficacy of rimonabant and BNS-002 in reversing HFD-induced kidney hyperfiltration. In addition, HFD-induced kidney hyperfiltration was completely normalized by BNS-002 (FIG. 10), suggesting increased ability of the novel compound to ameliorate obesity-induced kidney dysfunction.

FIGS. 11A-B demonstrate the efficacy of higher doses of BNS002 in DIO mice. The efficacy of higher doses of BNS-002 (15 and 30 mg/kg, ip for 7 days) was next tested in DIO mice in comparison with rimonabant (10 mg/kg/d). Age- and sex-matched mice on standard chow served as controls. The overweight of mice on HFD were significantly reduced by rimonabant and BNS-002 at a dose of 30 mg/kg (FIGS. 11A and 11B), whereas no effect on body weight reduction was observed in the group treated with BNS-002 at 15 mg/kg.

FIG. 12 provide Ki values determined for TMP using [³H]CP-55,940 radioligand displacement assay.

FIG. 13 provide Ki values determined for EST using [³H]CP-55,940 radioligand displacement assay.

FIG. 14 provide Ki values determined for IDB using [³H]CP-55,940 radioligand displacement assay.

FIG. 15 shows the ability of IDB, EST, TMP and rimonabant (as a positive control) to induce centrally-mediated hyperactivity in mice. Wild-type, male, C57Bl/6J mice received a single dose of rimonabant (10 mg/kg, IP), IDB, EST, TMP (at 20, 40 and 35 mg/kg, IP respectively) or vehicle. Ambulatory activity was measured by the Promethion Metabolic System (Sable Instruments, Inc). Data represent the mean±SEM from 4-8 mice per group. *P<0.05 vs. Vehicle-treated control.

FIG. 16 demonstrates the ability of IDB, EST, TMP and rimonabant (as a positive control) to inhibit the hypomotility-induced by a CB1 receptor agonist (HU210). Wild-type, male, C57Bl/6J mice received a single dose of rimonabant (10 mg/kg, IP), IDB, EST, TMP (at 20, 40 and 35 mg/kg IP, respectively) or vehicle. A half an hour after, mice received a single dose of HU210 (30 μg/kg, IP) and their locomotor activity was evaluated by the Promethion Metabolic System (Sable Instruments, Inc). Data represent the mean±SEM from 4-10 mice per group. *P<0.05 vs. Vehicle-treated control #P<0.05 vs. HU210.

FIGS. 17A-B show that IDB has a CB1 binding affinity of 256.3 nM (Ki) (A), and shows an inverse agonism profile, as tested by GTPγS binding (B). Data represent the mean±SEM of at least three independent experiments done in triplicates.

FIGS. 18A-F show that IDB (20 mg/kg/day for 20 days) reduced body weight (A, B), daily and total food intake (C, D) as well as reduced fat mas and increased lean mass (E, F) in DIO mice. Data represent the mean±SEM from 5 mice per group. *P<0.05 vs. Vehicle-treated control.

FIGS. 19A-F demonstrate that chronic IDB administration (20 mg/kg/day for 20 days) induces significant changes in metabolic parameters measured by the Promethion High-Definition Behavioral Phenotyping System (Sable Instruments, Inc.) over a 24 hr period. Respiratory quotient (A), VO2 (B), VCO2 (C), total energy expenditure (D), fat oxidation (E), and carbohydrate oxidation (F). Data are mean±SEM from 4 mice per group. *P<0.05 vs. Vehicle-treated control.

FIGS. 20A-D demonstrates that chronic IDB administration (20 mg/kg/day for 20 days) affects ambulation in DIO mice. Ambulatory activity (A), ability to run on a wheel (B), voluntary activity (C), and total meter (D). Method: Mice were monitored by the Promethion High-Definition Behavioral Phenotyping System (Sable Instruments, Inc.) over a 24 hr period. Data are mean±SEM from 4 mice per group.

FIGS. 21A-I show the effect of chronic IDB administration (20 mg/kg/day for 20 days) on glycemic control. Mice on high-fat diet for 20 weeks were treated chronically with IDB or vehicle, and glucose homeostasis was assessed. Note that IDB reduced glucose tolerance (A-B), improved insulin sensitivity (C—F) as well as reduced fasting (G) and fed (H) glucose levels. In addition, IDB increases glycosuria (I). Data represent the mean±SEM from 5 mice per group. *P<0.05 vs. Vehicle-treated control.

FIGS. 22A-B show that chronic IDB administration (20 mg/kg/day for 20 days) reduces HFD-induced hepatic steatosis and liver injury in mice. An elevated in fat vacuoles deposition, measured by H&E staining, was evident in the DIO mice treated with vehicle compared with the IDB-treated animals on the same diet (A). Furthermore, a decrease in liver weight (B) as well as a reduction in liver enzymes (AST, ALT, and ALP), measured by the COBAS Chemistry analyzer, was noticeable in the IDB-treated mice. Data represent the mean±SEM from 5 mice per group. *P<0.05 vs. Vehicle-treated control.

FIGS. 23A-E show that chronic IDB administration (20 mg/kg/day for 20 days) improves dyslipidemia in DIO mice. IDB was able to reduce total cholesterol (A), triglycerides (B), HDL (C), and LDL (D) as well as to increase HDL-to-LDL ratio (E). Data represent the mean±SEM from 5 mice per group. *P<0.05 vs. Vehicle-treated control.

### DETAILED DESCRIPTION OF THE INVENTION

As disclosed herein, “EST” is herein identified compound “I”. “TMP” is herein identified compound “H”. “IDB” is herein identified compound “K”. “BNS-002” is herein identified compound “0”.

### Synthesis and characterization of 5-(4-chlorophenyl)-1-(2,4-dichlorophenyl)-4-methyl-N-octadecyl-1H-pyrazole-3-carboxamide (BNS-002)

Synthesis procedure. A solution of Ethyl chloroformate (0.25 mL, 2.6 mmol) in dichloromethane (10 mL) were added to a 5-(4-chlorophenyl)-1-(2,4-dichlorophenyl)-4-methyl-1H-pyrazole-3-carboxylic acid (1 g, 2.6 mmol) in dry THF (150 mL). The mixture was added to a solution of stearylamine (0.7 g, 2.6 mmol) and triethylamine (0.38 ml, 2.8 mmol) in dry THF (200 mL). The addition performed slowly and in drop-wise at room temperature, rate 10 ml/min. The reaction mixture was stirred at room temperature over 4 hours. A pale-yellow solution and a white precipitate were formed. The mixture was filtered on white paper filter and washed with dry THF (50 ml). Following filtration, the THF was evaporated, and the crude was dissolved in hexane (150 ml), poured into separatory funnel, washed with DDW (100 ml) three times. The hexane layer was collected and dried over anhydrous sodium sulfate, filtered through white paper filter, and removed via evaporation forming a pale-yellow liquid. A 70% yield before column chromatography was obtained. The precipitate was dissolved again in 10 ml of dichloromethane and incorporated with silica powder (silica gel 60), dried and load to pre-prepared silica column (radius 5 cm, length 25 cm). The separation and the purification were completed as follows: 2 fold volumes of column capacity were washed with hexane; followed by 2 volumes of column capacity with hexane.

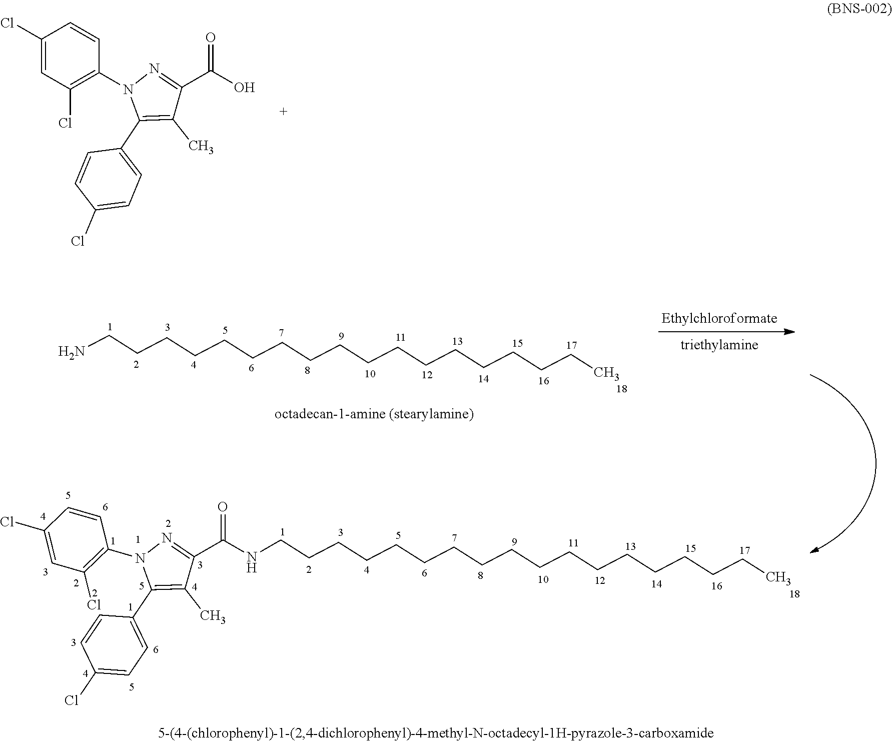 [SMILES: Cc1c(C(=O)O)nn(-c2ccc(Cl)cc2Cl)c1-c1ccc(Cl)cc1 ; CCCCCCCCCCCCCCCCCCN ; CCCCCCCCCCCCCCCCCCNC(=O)c1nn(-c2ccc(Cl)cc2Cl)c(-c2ccc(Cl)cc2)c1C]

Characterization. The LC-MS and the H-NMR spectrum confirmed the structure of the title compound. The HPLC shows purity above 98%.

Compounds having longer or shorter alkyl chains may be similarly prepared. Non-limiting examples of such compounds include:

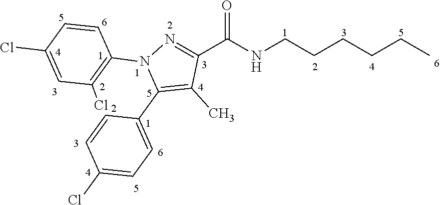 [SMILES: CCCCCCNC(=O)c1nn(-c2ccc(Cl)cc2Cl)c(-c2ccc(Cl)cc2)c1C]

5-(4-chlorophenyl)-1-(2,4-dichlorophenyl)-N-hexyl-4-methyl-1H-pyrazole-3-carboxamide

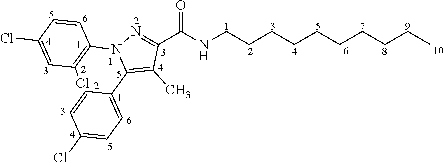 [SMILES: CCCCCCCCCCNC(=O)c1nn(-c2ccc(Cl)cc2Cl)c(-c2ccc(Cl)cc2)c1C]

5-(4-chlorophenyl)-N-decyl-1-(2,4-dichlorophenyl)-4-methyl-1H-pyrazole-3-carboxamide

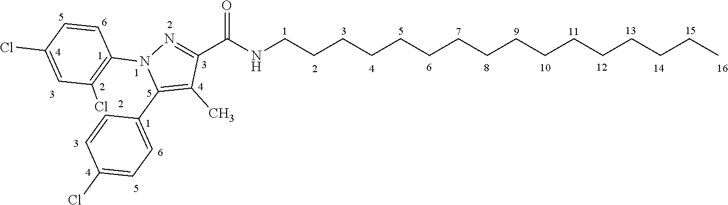 [SMILES: CCCCCCCCCCCCCCCCNC(=O)c1nn(-c2ccc(Cl)cc2Cl)c(-c2ccc(Cl)cc2)c1C]

5-(4-chlorophenyl)-1-(2,4-dichlorophenyl)-N-hexadecyl-4-methyl-1H-pyrazole-3-carboxamide

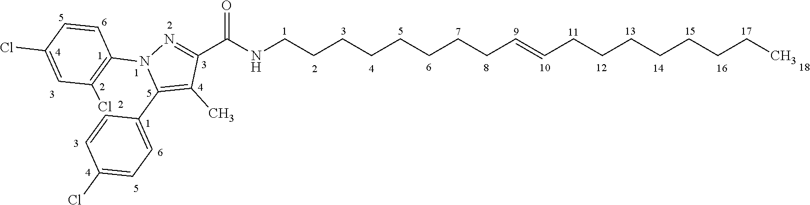 [SMILES: CCCCCCCC/C=C/CCCCCCCCNC(=O)c1nn(-c2ccc(Cl)cc2Cl)c(-c2ccc(Cl)cc2)c1C]

(E)-5-(4-chlorophenyl)-1-(2,4-dichlorophenyl)-4-methyl-N-(octadec-9-en-1-yl)-1H-pyrazole-3-carboxamide

Radioligand binding assays. BNS-002 binding to CB1 receptor was assessed in competition displacement assays using [³H]CP-55,940 as the radioligand and crude membranes from mouse brain for CB1 receptor. All data were in triplicates with Ki values determined from three independent experiments.

[³⁵S]GTPγS binding. Mouse brains were dissected and P2 membranes prepared and resuspended at ˜6 μg protein/μL in 1 ml assay buffer (50 mM Tris HCl, 9 mM MgCl2, 0.2 mM EGTA, 150 mM NaCl; pH 7.4). Ligand-stimulated [³⁵S]GTPγS binding was assayed as described previously (Tam et al., JCI 2010). Briefly, membranes (10 μg protein) were incubated in assay buffer containing 100 PM GDP, 0.05 nM [³⁵S]GTPγS, test compounds at 1 nM-1 μM, and 1.4 mg/mL fatty acid-free BSA in siliconized glass tubes. Bound ligand was separated from free by vacuum filtration. Non-specific binding was determined using 10 μM GTPS. Basal binding was assayed in the absence of the ligand and in the presence of GDP.

Tissue levels of antagonists. Mice received a single dose (3 or 10 mg/kg ip) of BNS-002 or rimonabant and were sacrificed 1 hour later. Blood was collected, and the mice were perfused with phosphate buffered saline for 1 min to remove drug from the intravascular space before removing the brain and liver. Drug levels in tissue homogenates and plasma were determined by using LC-MS/MS.

Locomotor activity. Locomotor activity was quantified by the number of disruptions of infrared XYZ beam arrays with a beam spacing of 0.25 cm in the Promethion High-Definition Behavioral Phenotyping System (Sable Instruments, Inc., Las Vegas, NV, USA).

Mice. The experimental protocol used was approved by the Institutional Animal Care and Use Committee of the Hebrew University, which is an AAALAC International accredited institute. Male 6 week old C57Bl/6J mice were obtained from Harlan Laboratories. Mice were maintained under a 12-h light/dark cycle and fed ad libitum. To generate diet-induced obesity, C57Bl6/J mice were fed either a high-fat diet (HFD) (60% of calories from fat, 20% from protein, and 20% from carbohydrates; Research Diet, D12492) or a standard laboratory diet (STD, 14% fat, 24% protein, 62% carbohydrates; NIH-31 rodent diet) for 14 weeks.

HFD-fed obese mice received vehicle (1% Tween80, 4% DMSO, 95% Saline), BNS-002, IDB or rimonabant daily for 7-28 days by intraperitoneal (ip) injections of 10, 15, 20, and 30 mg/kg as indicated in the figures. Age-matched control mice on STD received vehicle daily. Body weight and food intake were monitored daily. Total body fat and lean masses were determined by EchoMRI-100H™ (Echo Medical Systems LLC, Houston, TX, USA). 24 h urine was collected one week before euthanasia using mouse metabolic cages (CCS2000 Chiller System, Hatteras Instruments, NC, USA). At weeks 20 mice were euthanized by a cervical dislocation under anesthesia, the kidneys, brain, liver, fat pads, and muscles were removed and weighed, and samples were either snap-frozen or fixed in buffered 4% formalin. Trunk blood was collected for determining the biochemical parameters.

Multi-parameter metabolic assessment. Metabolic profile of the mice was assessed by using the Promethion High-Definition Behavioral Phenotyping System (Sable Instruments, Inc., Las Vegas, NV, USA). Data acquisition and instrument control were performed using MetaScreen software version 2.2.18.0, and the obtained raw data were processed using ExpeData version 1.8.4 using an analysis script detailing all aspects of data transformation. Mice with free access to food and water were subjected to a standard 12 h light/12 h dark cycle, which consisted of a 48 h acclimation period followed by 24 h of sampling. Respiratory gases were measured by using the GA-3 gas analyzer (Sable Systems, Inc., Las Vegas, NV, USA) using a pull-mode, negative-pressure system. Air flow was measured and controlled by FR-8 (Sable Systems, Inc., Las Vegas, NV, USA), with a set flow rate of 2000 mL/min. Water vapor was continuously measured and its dilution effect on O₂ and CO₂ was mathematically compensated. Effective mass was calculated by [body mass]^{0.75}. Fat oxidation (FO) and carbohydrate oxidation (CHO) were calculated as FO=1.69×VO₂−1.69×VCO₂ and CHO=4.57×VCO₂—3.23×VO₂ and expressed as g/d/kg^{eff.Mass}.

Glucose tolerance (ipGTT) test and insulin sensitivity tests (ipIST). Mice that fasted overnight were injected with glucose (1.5 g/kg, ip), followed by a tail blood collection at 0, 15, 30, 45, 60, 90, and 120 minutes. Blood glucose levels were determined using the Elite glucometer (Bayer, Pittsburgh, PA). On the following day, mice were fasted for 6 h before receiving insulin (0.75 U/kg, i.p.; Eli Lilly, DC, USA or Actrapid® vial, novo nordisk A/S, Denmark), and blood glucose levels were determined at the same intervals as above.

Blood and urine biochemistry. Serum and urine levels of creatinine as well as serum levels of ALT, AST, ALP, HDL, LDL, TG and cholesterol were determined by using the Cobas C-111 chemistry analyzer (Roche, Switzerland). Creatinine clearance was calculated using urine and serum creatinine levels (CCr mL/h=Urine creatinine mg/dL×Urine volume/Serum creatinine mg/dL×24 hrs). Serum insulin levels were measured by an ELISA kit (Crystal Chem, Inc., Downers Grove, IL, USA). Fasting blood glucose was measured using the Elite glucometer (Bayer, Pittsburgh, PA).

Histopathological Analyses. 5 μm paraffin-embedded liver sections from 5 animals per group were stained with hematoxylin-eosin staining. Liver images were captured with a Zeiss AxioCam ICc5 color camera mounted on a Zeiss Axio Scope.A1 light microscope and taken from 10 random 40× fields of each animal.

### Results:

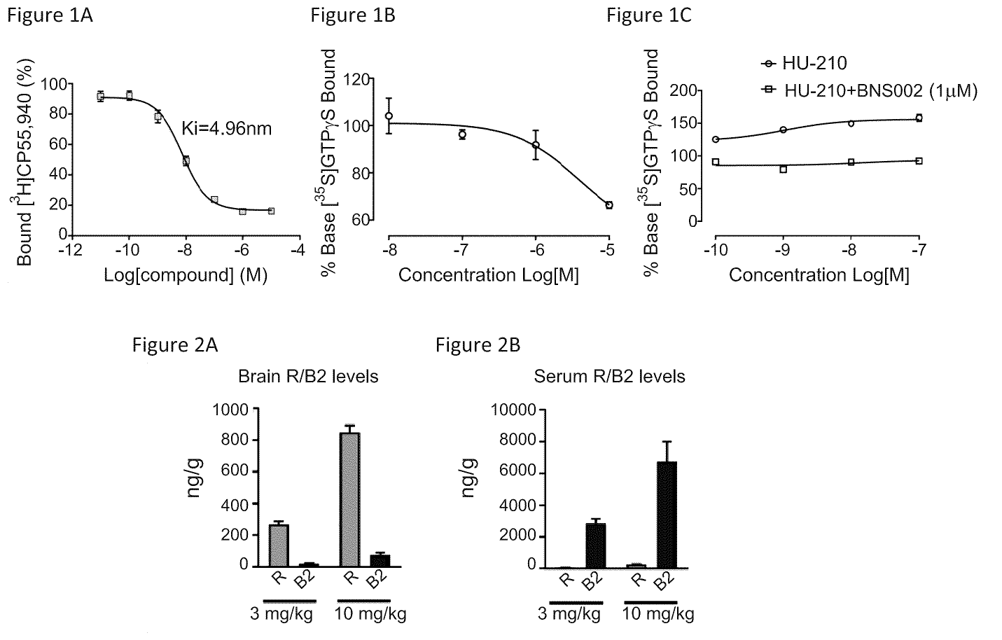

BNS-002 is more lipid soluble than rimonabant (estimated partition coefficient [log P], 17 vs. 6.4 for rimonabant) but retains high affinity and selectivity for CB1 receptor. In radioligand displacement assays, BNS-002 has a Ki of 4.96 nM for CB1 receptor, which is similar to that of rimonabant (FIG. 1A). Like rimonabant, BNS-002 reduces GTPγS binding in mouse brain membranes (FIG. 1B) and is able to ameliorate the action of the potent CB1 receptor agonist HU-210 (FIG. 1C), suggesting that it is an inverse agonist.

Importantly, BSN002 displays markedly reduced brain penetrance, as reflected by its reduced brain levels and increased serum levels following an administration of the compound in two different doses (3 and 10 mg/kg, ip; FIGS. 2A-B).

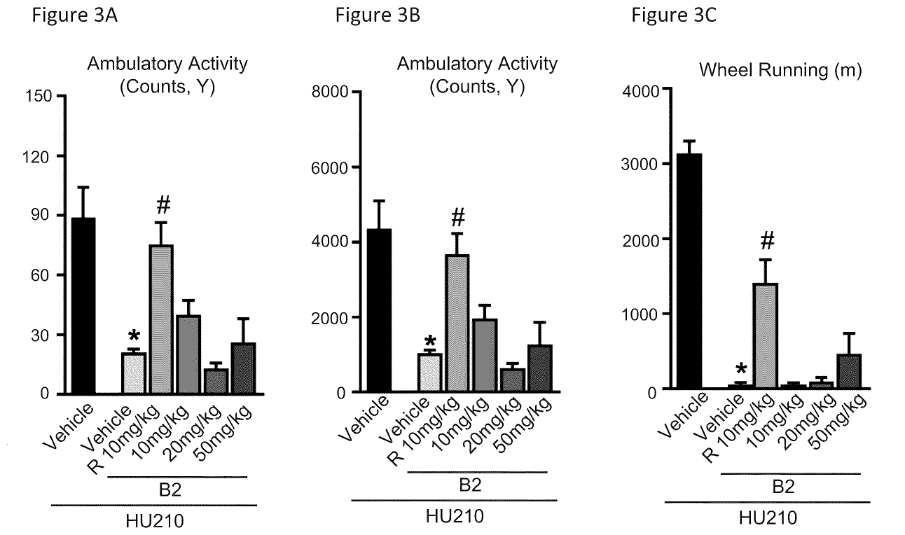

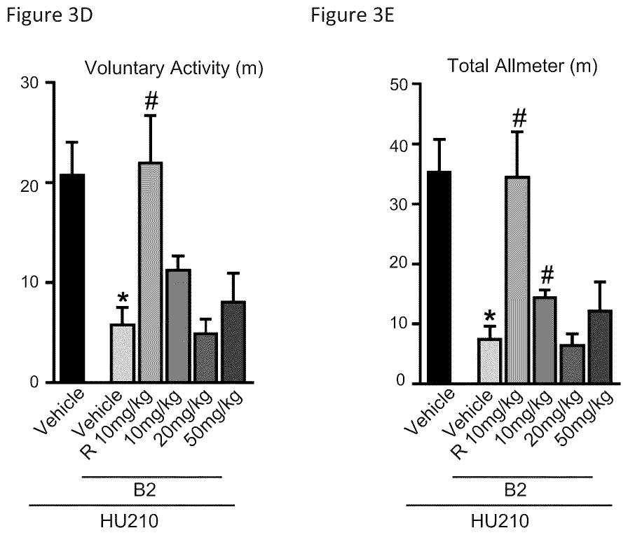

Next the inventors tested whether the reduced brain penetrance of BNS-002 is associated with an attenuation of behavioral effects. To that end, we compared the effects of BNS-002 and rimonabant in antagonizing cannabinoid-induced hypomotility. The marked increase in immobility induced in mice by the cannabinoid agonist HU-210 (30 μg/kg, ip) was completely blocked by rimonabant (10 mg/kg, ip) but was unaffected by a similar dose and even higher doses of BNS-002 (10, 20, and 50 mg/kg; FIGS. 3A-E).

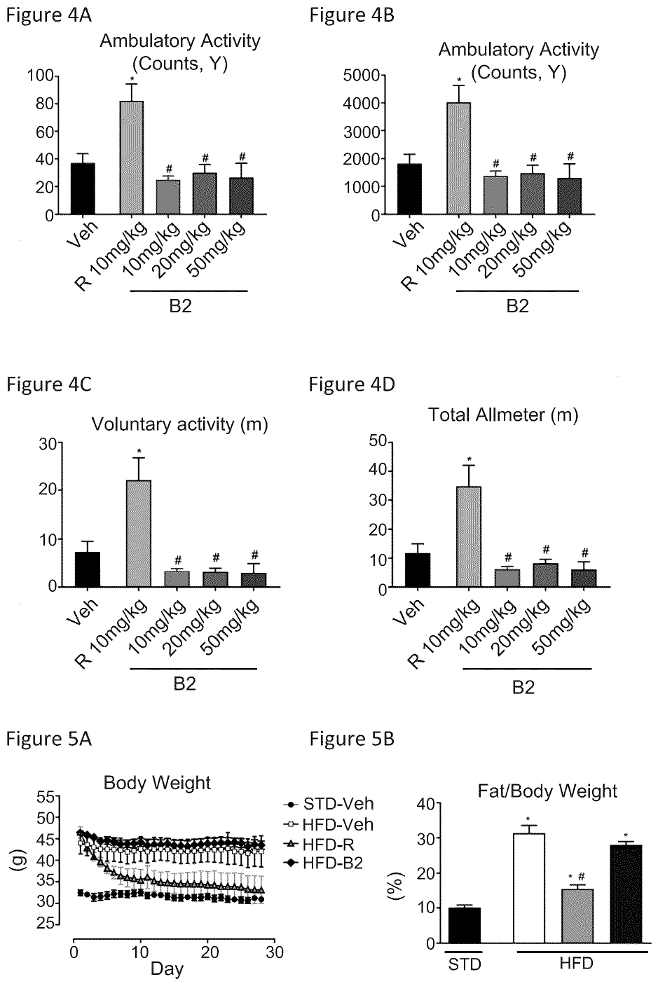

In addition, rimonabant (10 mg/kg, ip), but not BNS-002 (at 10, 20 and 50 mg/kg, ip), also induced a marked increase in the activity profile in mice (FIGS. 4A-D).

The metabolic profile of BNS-002 and rimonabant was next examined in mice with diet-induced obesity (DIO). Male C57BL/6 mice fed a high-fat diet (HFD) for 14 weeks became obese and were then started on daily ip injections of vehicle, rimonabant, or AM6545 (both at 10 mg/kg/d) for an additional 28 days. Age- and sex-matched mice on standard chow served as controls. The overweight and increased adiposity of mice on HFD were significantly reduced by rimonabant only (FIGS. 5A-B).

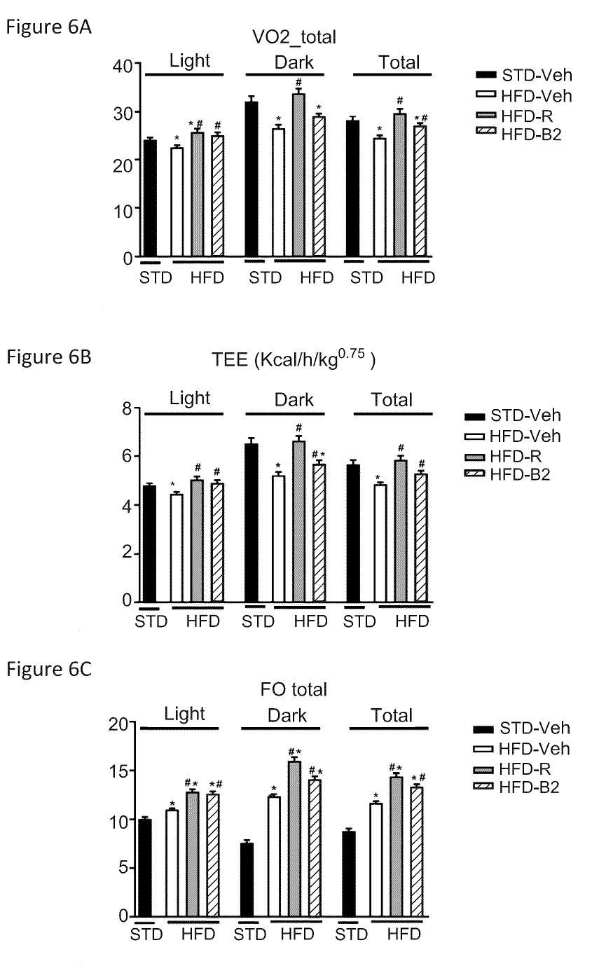

Yet, significant increase in the metabolic profile of the DIO mice treated with both antagonists was demonstrated using an indirect calorimetry assessment. As shown in FIGS. 6A-C, both rimonabant and BNS-002 were able to upregulate the HFD-induced reduction in VO₂, total energy expenditure, and fat oxidation.

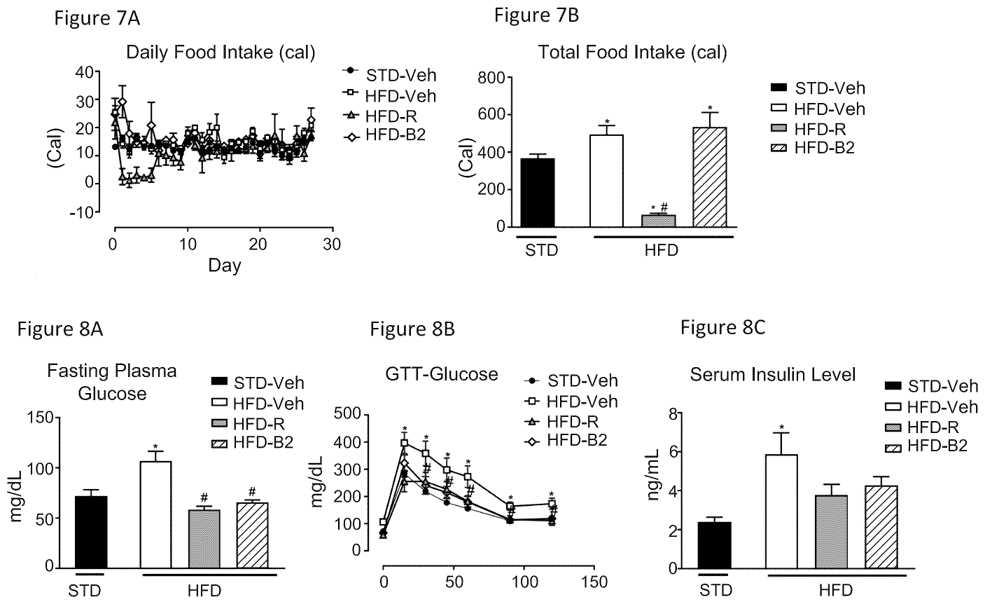

The greater efficacy of rimonabant over BNS-002 in reducing body weight is probably related to its ability to reduce total caloric intake (FIGS. 7A-B).

Nevertheless, HFD-induced hyperglycemia and glucose intolerance were completely reversed by BNS-002 in a similar fashion as rimonabant (FIGS. 8A-B). A trend toward reduction in serum insulin levels was also documented by both compounds (FIG. 8C).

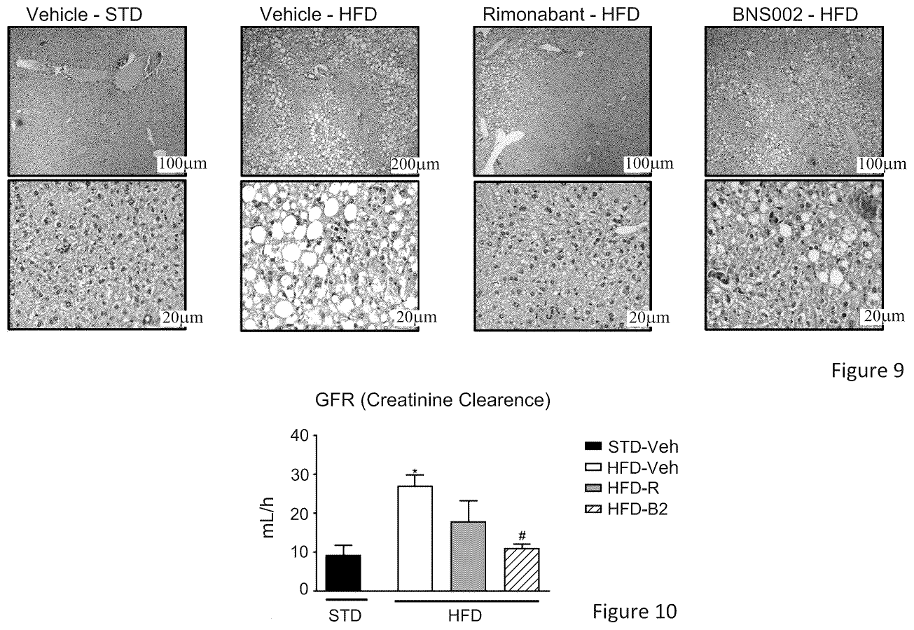

Moreover, HFD-induced hepatic steatosis, as reflected in elevated fat vacuoles in the liver, was completely reversed by rimonabant and partially by BNS-002 (FIG. 9).

In addition, HFD-induced kidney hyperfiltration was completely normalized by BNS-002 (FIG. 10), suggesting increased ability of the novel compound to ameliorate obesity-induced kidney dysfunction.

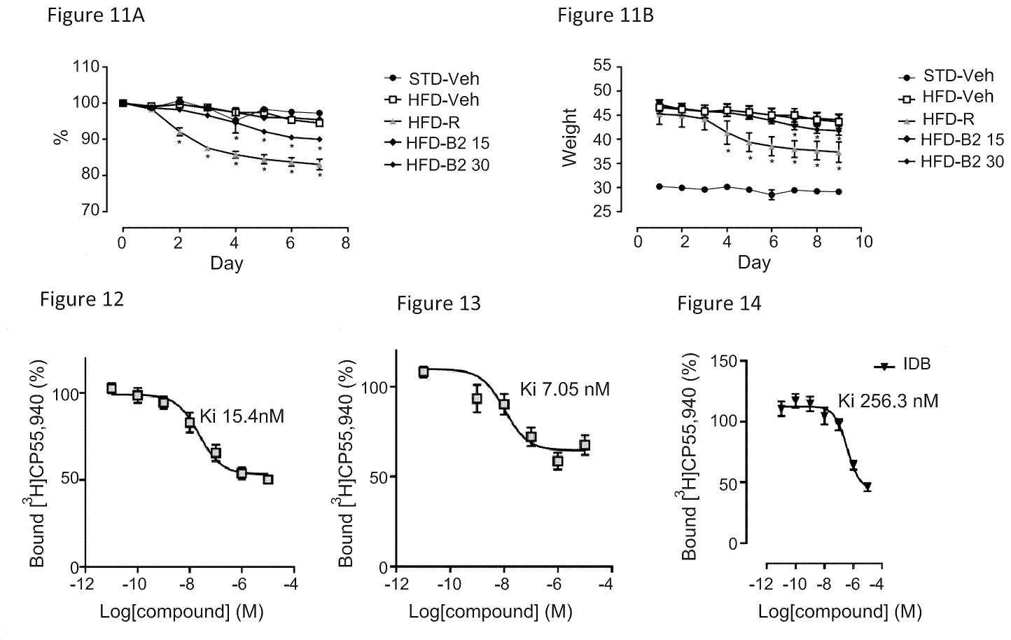

The efficacy of higher doses of BNS-002 (15 and 30 mg/kg, ip for 7 days) was next tested in DIO mice in comparison with rimonabant (10 mg/kg/d). Age- and sex-matched mice on standard chow served as controls. The overweight of mice on HFD were significantly reduced by rimonabant and BNS-002 at a dose of 30 mg/kg (FIGS. 11A and 11B), whereas no effect on body weight reduction was observed in the group treated with BNS-002 at 15 mg/kg.

### Synthesis and characterization of 5-(4-chlorophenyl)-1-(2,4-dichlorophenyl)-4-methyl-N-(2,2,6,6-tetramethyl-1-oxo-1-piperidin-4-yl)-1H-pyrazole-3-carboxamide (BB1+TMP)

Synthesis procedure. N,N′-Dicyclohexylcarbodiimide (DCC, 1.08 g, 5.24 mmol) was added to 5-(4-chlorophenyl)-1-(2,4-dichlorophenyl)-4-methyl-1H-pyrazole-3-carboxylic acid (BB1, 1 g, 2.26 mmol) in CH₂Cl₂ (70 ml). The resultant mixture was stirred for 10 min and then, 4-Amino TEMPO (free radical) (TMP, 0.45 g, 2.62 mmol) was added. The reaction mixture was stirred at room temperature over 24 h. An orange solution and a white precipitate were formed. The mixture was filtered on white paper filter and washed with CH₂Cl₂ (50 ml). Following filtration, the CH₂Cl₂ was evaporated, and the crude was dissolved in CH₂Cl₂ again (50 ml). An orange solution and a white precipitate were formed. The mixture was filtered on white paper filter and washed with CH₂Cl₂ (50 ml). Following filtration, the CH₂Cl₂ was evaporated.

A 74% yield before column chromatography was obtained. The orange viscous oil was dissolved again in 10 ml of CH₂Cl₂ and incorporated with silica powder (silica gel 60), dried and load to pre-prepared silica column (radius 5 cm, length 25 cm). The separation and the purification were completed as follows: 2 fold volumes of column capacity were washed with hexane; followed by 2 volumes of column capacity with hexane:ethyl acetate (90:10) and ended after 4 volumes of column capacity with hexane:ethyl acetate (80:20).

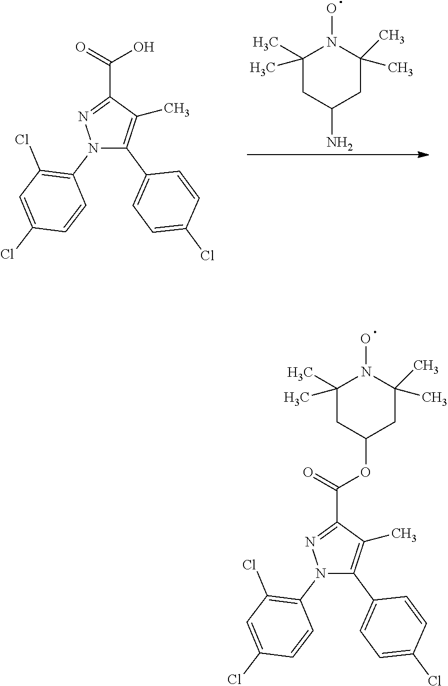

Characterization. The LC-MS and the Elemental analysis confirmed the structure of the title compound. The HPLC shows purity above 98%.

### Elemental Analysis

TABLE 1

| Batch | Code | | | |
|---|---|---|---|---|
| Sample | Sample | % C | % H | % N |
| 6-6956 | BB1 + TMP | 58.31 | 5.23 | 10.46 |
| | | 57.27 | 5.44 | 9.91 |

### Synthesis and characterization of 2,2,6,6-tetramethyl-1-piperidin-4-yl 5-(4-chlorophenyl)-1-(2,4-dichlorophenyl)-4-methyl-1H-pyrazole-3-carboxylate (BB1+EST)

Synthesis procedure. N,N′-Dicyclohexylcarbodiimide (DCC, 1.08 g, 5.24 mmol) was added to 5-(4-chlorophenyl)-1-(2,4-dichlorophenyl)-4-methyl-1H-pyrazole-3-carboxylic acid (BB1, 1 g, 2.26 mmol) in CH₂Cl₂ (70 ml). The resultant mixture was stirred for 10 min and then, 4-Hydroxy TEMPO (free radical) (EST, 0.45 g, 2.62 mmol) was added. The reaction mixture was stirred at room temperature over 24 h. An orange solution and a white precipitate were formed. The mixture was filtered on white paper filter and washed with CH₂Cl₂ (50 ml). Following filtration, the CH₂Cl₂ was evaporated, and the crude was dissolved in CH₂Cl₂ again (50 ml). An orange solution and a white precipitate were formed. The mixture was filtered on white paper filter and washed with CH₂Cl₂ (50 ml). Following filtration, the CH₂Cl₂ was evaporated.

A 70% yield before column chromatography was obtained. The orange viscous oil was dissolved again in 10 ml of CH₂Cl₂ and incorporated with silica powder (silica gel 60), dried and load to pre-prepared silica column (radius 5 cm, length 25 cm). The separation and the purification were completed as follows: 2 fold volumes of column capacity were washed with hexane; followed by 2 volumes of column capacity with hexane:ethyl acetate (90:10) and ended after 4 volumes of column capacity with hexane:ethyl acetate (80:20).

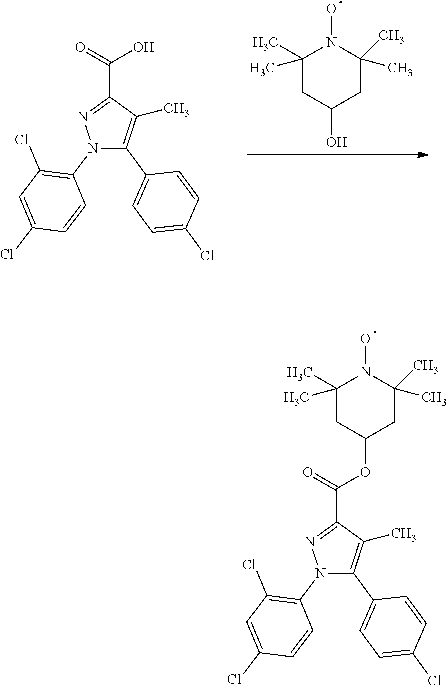

Characterization. The LC-MS and the Elemental analysis confirmed the structure of the title compound. The HPLC shows purity above 98%.

### Elemental Analysis

TABLE 2

| Batch | | Code | | | |
|---|---|---|---|---|---|
| Number | Date | Sample | % C | % H | % N |
| 6-7154 | | | 58.20 | 5.22 | 7.83 |
| | 30 Jul. 2019 | BB1_EST | 57.71 | 5.17 | 7.54 |

### Synthesis and characterization of 10-(4,5-dimethoxy-2-methyl-3,6-dioxocyclohexa-1,4-dien-1-yl)decyl 5-(4-chlorophenyl)-1-(2,4-dichlorophenyl)-4-methyl-1H-pyrazole-3-carboxylate (BB1+IDB)

Synthesis procedure. N,N′-Dicyclohexylcarbodiimide (DCC, 1.3 g, 5.91 mmol) was added to 5-(4-chlorophenyl)-1-(2,4-dichlorophenyl)-4-methyl-1H-pyrazole-3-carboxylic acid (BB1, 1.12 g, 2.95 mmol) in CH₂Cl₂ (70 ml). The resultant mixture was stirred for 10 min and then, Idebenone (IDB, 1, 2.95 mmol) was added. The reaction mixture was stirred at room temperature over 24 h. An orange solution and a white precipitate were formed. The mixture was filtered on white paper filter and washed with CH₂Cl₂ (50 ml). Following filtration, the CH₂Cl₂ was evaporated, and the crude was dissolved in CH₂Cl₂ again (50 ml). An orange solution and a white precipitate were formed. The mixture was filtered on white paper filter and washed with CH₂Cl₂ (50 ml). Following filtration, the CH₂Cl₂ was evaporated.

A 70% yield before column chromatography was obtained. The orange viscous oil was dissolved again in 10 ml of CH₂Cl₂ and incorporated with silica powder (silica gel 60), dried and load to pre-prepared silica column (radius 5 cm, length 25 cm). The separation and the purification were completed as follows: 2 fold volumes of column capacity were washed with hexane; followed by 2 volumes of column capacity with hexane:ethyl acetate (90:10) and ended after 4 volumes of column capacity with hexane:ethyl acetate (80:20).

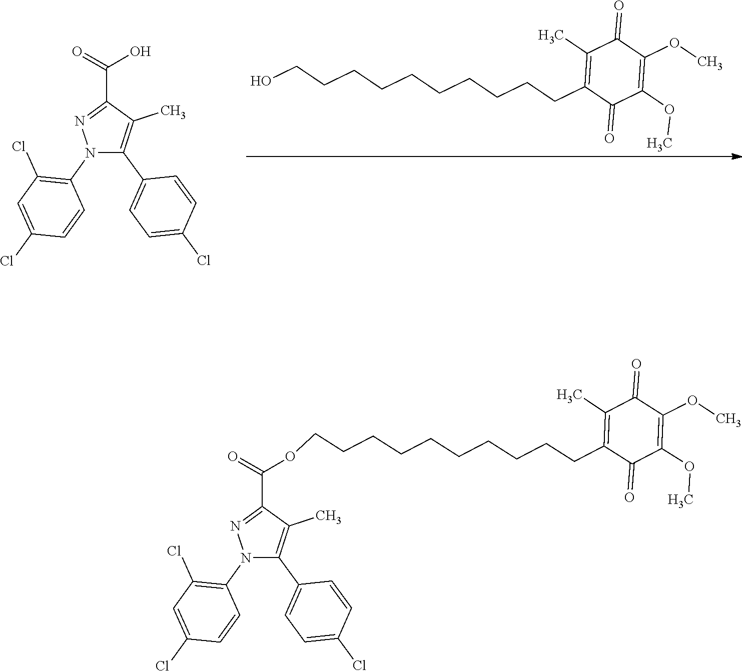 [SMILES: Cc1c(C(=O)O)nn(-c2ccc(Cl)cc2Cl)c1-c1ccc(Cl)cc1 ; COC1=C(OC)C(=O)C(CCCCCCCCCCO)=C(C)C1=O ; COC1=C(OC)C(=O)C(CCCCCCCCCCOC(=O)c2nn(-c3ccc(Cl)cc3Cl)c(-c3ccc(Cl)cc3)c2C)=C(C)C1=O]

Characterization. The LC-MS and the H-NMR confirmed the structure of the title compound. The HPLC shows purity above 98%.

### TMP, EST and IDB—In Vitro Binding Report

Radioligand binding assay. Binding of the tested compounds to CB1 receptor was assessed in competition displacement assays using [3H]CP-55,940 as the radioligand and crude membranes from mouse brain for CB1 receptor. Membranes were extracted according to an established protocol previously described by Catani V. M. and Gasperi V. [8]. Compounds were tested at different concentrations (10-5M-10-11M) and their ability to displace [3H]CP-55,940 was evaluated. Membranes with bound [3H]CP-55,940 were separated and washed from free ligand by vacuum filtration and bound [3H]CP-55,940 radioactivity was measured using a p counter. All data were in triplicates with Ki values extracted by nonlinear regression analysis using GraphPad Prism software.

### Results

In radioligand displacement assays, all three tested compounds were found active with high affinity to CB1 receptor. Ki values were varying for each substance, ranging from 1.69 nM-446 nM for TMP (FIG. 12), 0.37 nM-7.81 nM for EST (FIG. 13) and 1.9 nM-134.6 nM for IDB (FIG. 14).

### TMP, EST and IDB—In Vivo Safety Report (Lack of CNS Central Activity)

Centrally-mediated hyperactivity profile. Wild-type, male, C57Bl/6J mice (n=4-8) received a single dose of rimonabant (10 mg/kg, IP), TMP (35 mg/kg, IP), EST (40 mg/kg, IP), IDB (20 mg/kg, IP) or vehicle only (IP). Mice were placed in metabolic cages and their activity profile was evaluated. Locomotor activity was quantified by the number of disruptions of infrared XYZ beam arrays with a beam spacing of 0.25 cm in the Promethion High-Definition Behavioral Phenotyping System (Sable Instruments, Inc., Las Vegas, NV, USA).

Antagonizing cannabinoid-induced hypomotility. The ability of the different compounds to inhibit the hypomotility induced by HU210 (cannabinoid agonist) was evaluated. Wild-type, male, C57Bl/6J mice (n=4-10) received a single dose of rimonabant (10 mg/kg, IP), TMP (35 mg/kg, IP), EST (40 mg/kg, IP), IDB (20 mg/kg, IP) or vehicle only (IP). A half an hour thereafter, mice received a single dose of HU210 (30 ug/kg, IP) and their locomotor activity was evaluated as described above.

### Results

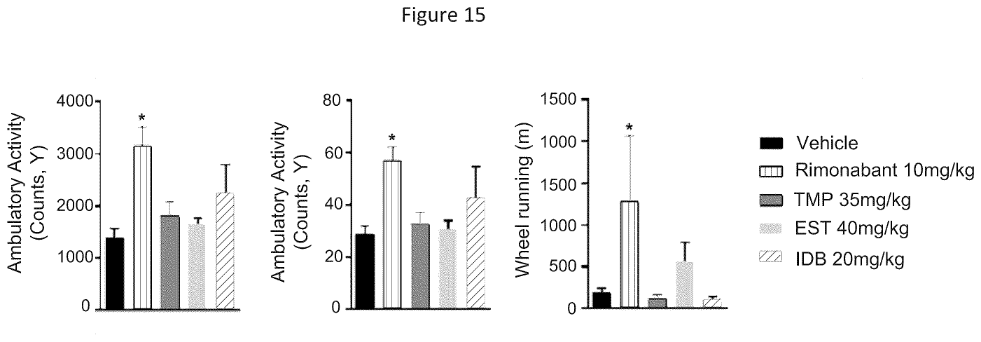

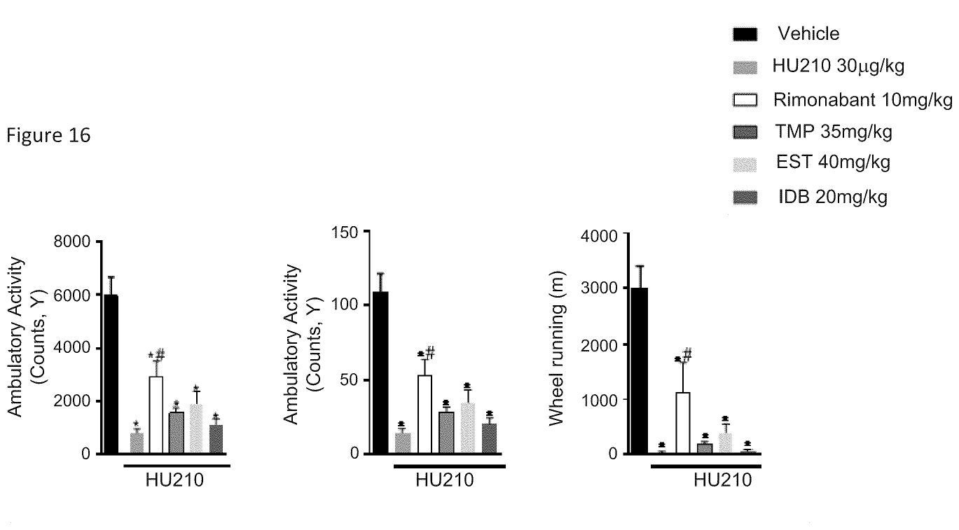

Rimonabant (10 mg/kg) induced a marked increase in the activity profile in mice (FIG. 15), but no significant hyperactivity was recorded, compare to the vehicle group, following TMP (35 mg/kg, IP), EST (40 mg/kg, IP) and IDB (20 mg/kg, IP) injections (FIG. 15). The marked hypomotility induced in mice by the cannabinoid agonist HU210 (30 ug/kg, IP) was significantly blocked by rimonabant but was unaffected by the tested compounds (FIG. 16).

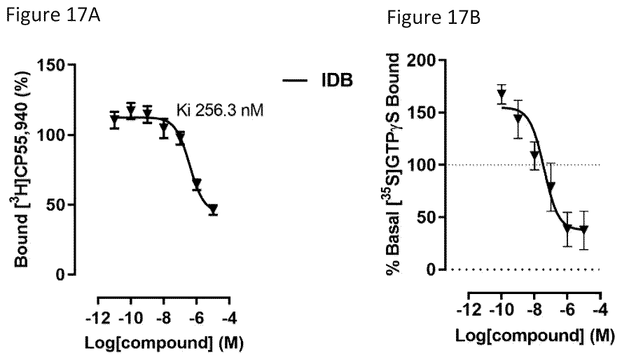

FIG. 17 shows that IDB has a CB1 binding affinity of 256.3 nM (Ki) (A), and shows an inverse agonism profile, as tested by GTPγS binding (B). Data represent the mean±SEM of at least three independent experiments done in triplicates.

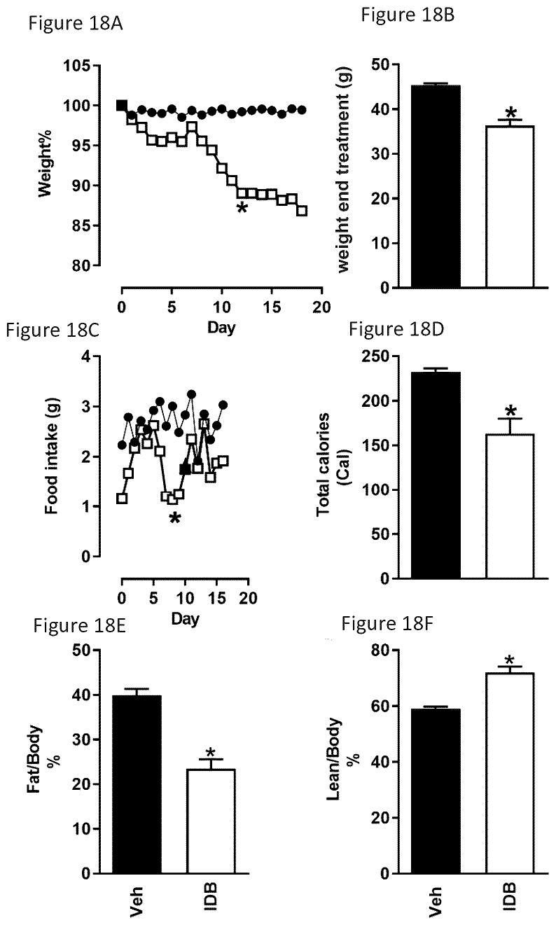

IDB (20 mg/kg/day for 20 days) reduced body weight (A, B), daily and total food intake (C, D) as well as reduced fat mas and increased lean mass (E, F) in DIO mice is shown in FIG. 18. Data represent the mean±SEM from 5 mice per group. *P<0.05 vs. Vehicle-treated control.

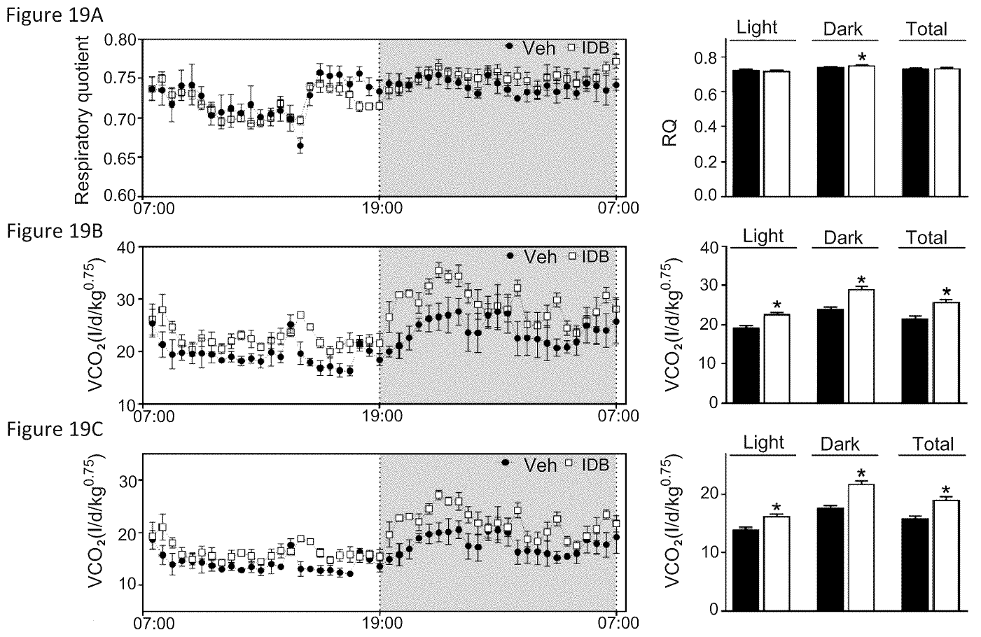

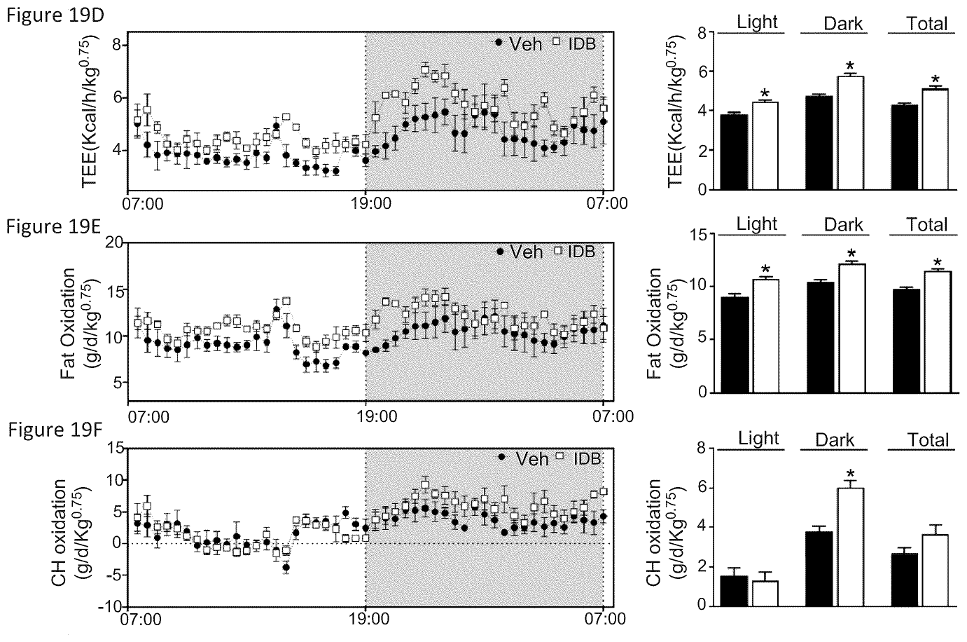

In FIG. 19 chronic IDB administration (20 mg/kg/day for 20 days) is shown to induce significant changes in metabolic parameters measured by the Promethion High-Definition Behavioral Phenotyping System (Sable Instruments, Inc.) over a 24 hr period. Respiratory quotient (A), VO2 (B), VCO2 (C), total energy expenditure (D), fat oxidation (E), and carbohydrate oxidation (F). Data are mean±SEM from 4 mice per group. *P<0.05 vs. Vehicle-treated control.

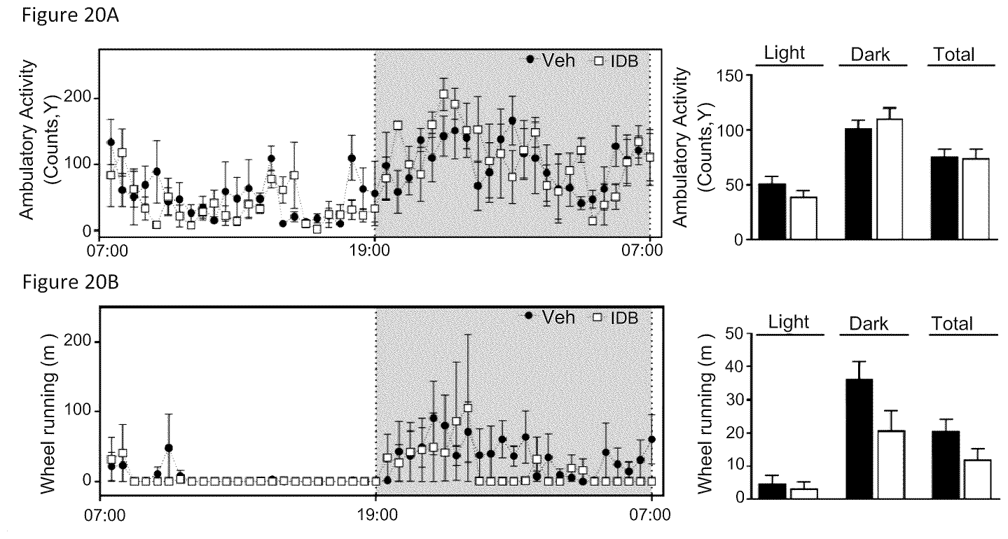

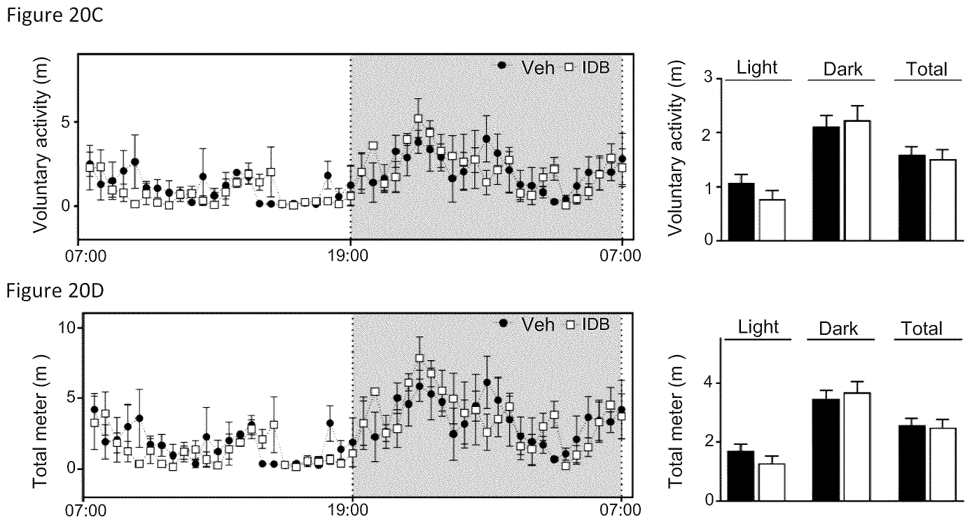

In FIG. 20 chronic IDB administration (20 mg/kg/day for 20 days) is shown not to affect ambulation in DIO mice. Ambulatory activity (A), ability to run on a wheel (B), voluntary activity (C), and total meter (D). Method: Mice were monitored by the Promethion High-Definition Behavioral Phenotyping System (Sable Instruments, Inc.) over a 24 hr period. Data are mean±SEM from 4 mice per group.

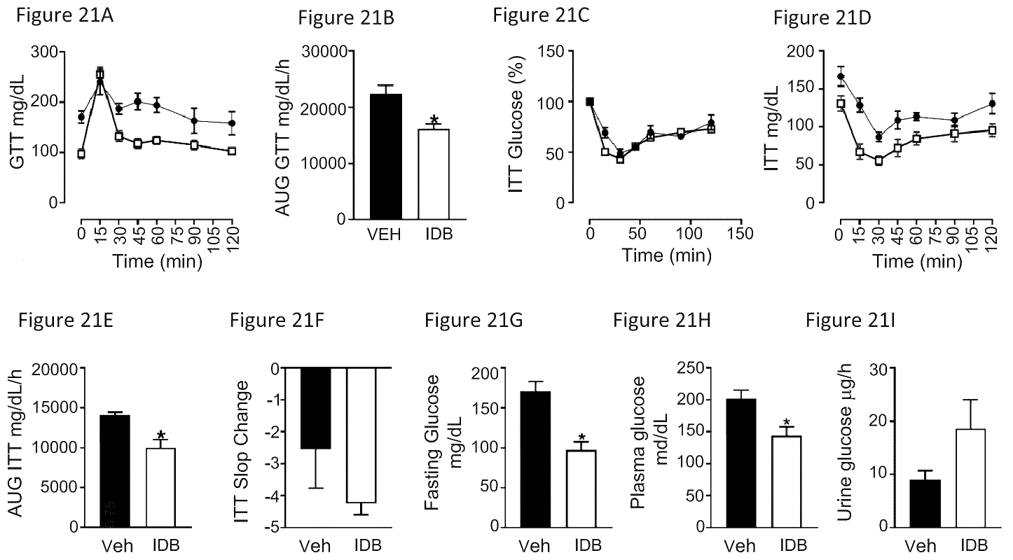

In FIG. 21 the effect of chronic IDB administration (20 mg/kg/day for 20 days) on glycemic control is demonstrated. Mice on high-fat diet for 20 weeks were treated chronically with IDB or vehicle, and glucose homeostasis was assessed. Note that IDB reduced glucose tolerance (A-B), improved insulin sensitivity (C—F) as well as reduced fasting (G) and fed (H) glucose levels. In addition, IDB increases glycosuria (I). Data represent the mean±SEM from 5 mice per group. *P<0.05 vs. Vehicle-treated control.

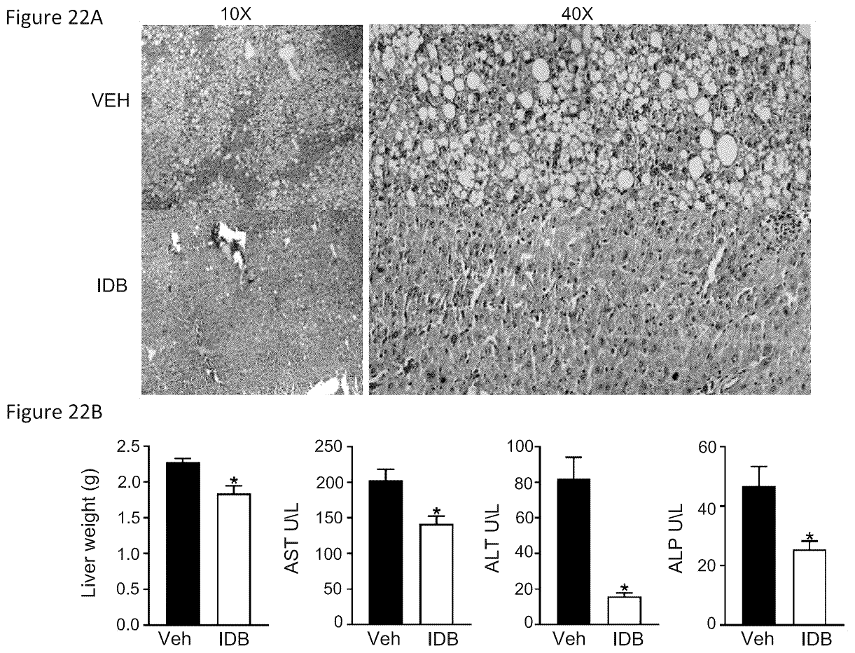

In FIG. 22 chronic IDB administration (20 mg/kg/day for 20 days) is shown to reduce HFD-induced hepatic steatosis and liver injury in mice. An elevated in fat vacuoles deposition, measured by H&E staining, was evident in the DIO mice treated with vehicle compared with the IDB-treated animals on the same diet (A).

Furthermore, a decrease in liver weight (B) as well as a reduction in liver enzymes (AST, ALT, and ALP), measured by the COBAS Chemistry analyzer, was noticeable in the IDB-treated mice. Data represent the mean±SEM from 5 mice per group. *P<0.05 vs. Vehicle-treated control.

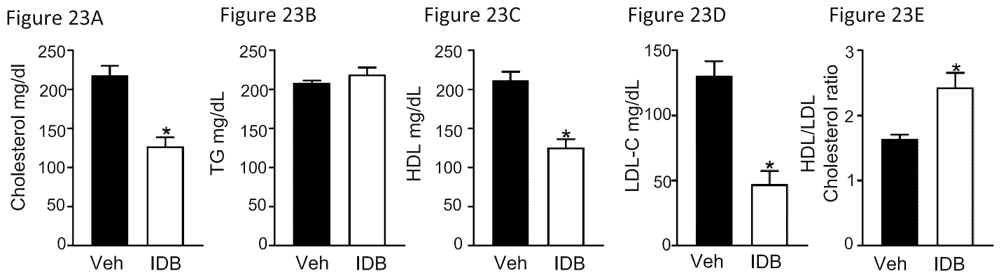

In FIG. 23 chronic IDB administration (20 mg/kg/day for 20 days) is shown to improve dyslipidemia in DIO mice. IDB was able to reduce total cholesterol (A), triglycerides (B), HDL (C), and LDL (D) as well as to increase HDL-to-LDL ratio (E). Data represent the mean±SEM from 5 mice per group. *P<0.05 vs. Vehicle-treated control.

One very important difference between BNS-002 and IDB reside in the different impact on the liver and kidney functions. As can be seen in FIG. 10, HFD-induced kidney hyperfiltration was completely normalized by BNS-002, suggesting increased ability of the novel compound to ameliorate obesity-induced kidney dysfunction. Whereas IDB has no effect compared to the control. Furthermore, HFD-induced hepatic steatosis, as reflected in elevated fat vacuoles in the liver, was completely reversed by rimonabant and partially by BNS-002 (FIG. 9). Whereas chronic IDB administration (20 mg/kg/day for 20 days) of IDB reduces HFD-induced hepatic steatosis and liver injury in mice. An elevated in fat vacuoles deposition, measured by H&E staining, was evident in the DIO mice treated with vehicle compared with the IDB-treated animals on the same diet (FIG. 22A). Furthermore, a decrease in liver weight (FIG. 22B) as well as a reduction in liver enzymes (AST, ALT, and ALP), measured by the COBAS Chemistry analyzer, was noticeable in the IDB treated mice. Data represent the mean±SEM from 5 mice per group. *P<0.05 vs. Vehicle-treated control.

1-234. (canceled)

235. A compound of the general formula (II): 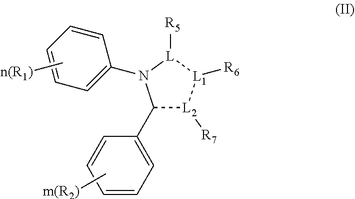
wherein
each of R₁ and R₂, independently of the other, is a group selected from —H, halide, —CN, —C₁-C₅alkyl-OH and —OH;
each of n and m, independently of the other, is an integer between 0 and 5, designating the number of substituents on the ring;
one of L, L₁ and L₂ is a nitrogen atom and the others of L, L₁ and L₂ are each a carbon atom;
each of R₅, R₆ and R₇, independently of the other, is selected from —H, —C₁-C₃alkyl, —C(═O)—OH, —C(═O)—O—R₈, —C(═O)—NR′R₈, halide, —CN, —OH, and —NR′R″; or
one of R₅ and R₆ or R₆ and R₇ together with the atoms to which they bond form a 5-, 6-, 7- or 8-membered carbocyclic ring optionally containing between 1 and 3 heteroatoms selected from N, O and S; or
one of R₅, R₆ and R₇ is absent;
the 5-, 6-, 7- or 8-membered carbocyclic ring is optionally substituted by at least one functionality selected from —H, —C₁-C₂₅alkyl, —C₂-C₂₅alkenyl, —C₂-C₂₅alkynyl, —C₆-C₁₀aryl, an hydroxyl, an amine, a halide, —ONO₂, —NO₂, —S—, —S—C₁-C₅alkyl, —S—C₂-C₅alkenyl, —S—C₂-C₅alkynyl, —C(═O)—, —C(═O)—C₁-C₂₅alkyl, —C(═O)—O—C₁-C₅alkyl, —C(═O)—O—C₂-C₅alkenyl, —C(═O)—O—C₂-C₅alkynyl, —C(═O)—NR′R″R′″, —C(═O)—NR′—C(═O)—C₁-C₂₅alkyl, —C(═O)—NR′—C(═O)—C₂-C₂₅alkenyl, —C(═O)—NR′—C(═O)—C₂-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″;
the 5-, 6-, 7- or 8-membered carbocyclic ring is optionally substituted by at least one functionality selected from structures (A) through (H): 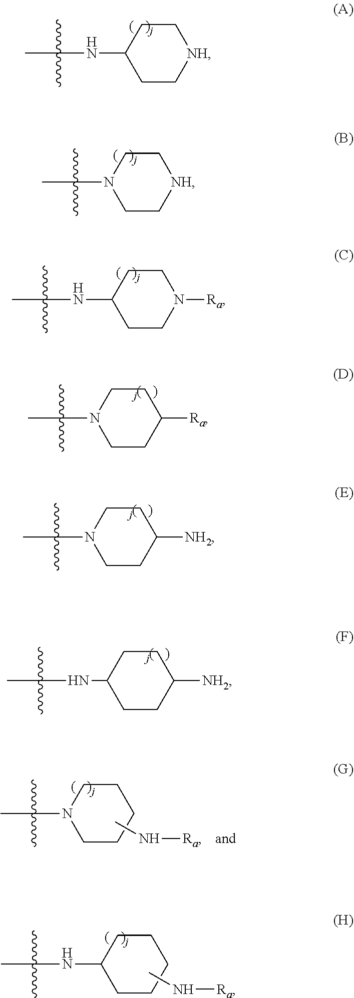
wherein in each functionality (A) through (H), the wavy line indicates point or bond of connectivity, j is 0 or 1 and Ra is selected from —H, —C₁-C₂₅alkyl, —C₂-C₂₅alkenyl, —C₂-C₂₅alkynyl, —C(═O)—C₆-C₁₀aryl and —C(═O)—C₃-C₁₀heteroaryl, wherein in functionalities (G) and (H) the pendant —NH—Ra group may appears between 1 and 11 times at any position along the carbocycle;
R₈ is selected from —H, —C₁-C₂₅alkyl, —C₂-C₂₅alkenyl, —C₂-C₂₅alkynyl, —C₆-C₁₀aryl and C₃-C₁₀heteroaryl, each of which being optionally substituted by at least one functionality selected from an hydroxyl, an amine, a halide, —ONO₂, —NO₂, —S—, —S—C₁-C₅alkyl, —S—C₂-C₅alkenyl, —S—C₂-C₅alkynyl, —C(═O)—, —C(═O)—C₁-C₂₅alkyl, —C(═O)—O—C₁-C₅alkyl, —C(═O)—O—C₂-C₅alkenyl, —C(═O)—O—C₂-C₅alkynyl, —C(═O)—NR′R″R′″, —C(═O)—NR′—C(═O)—C₁-C₂₅alkyl, —C(═O)—NR′—C(═O)—C₁-C₂₅alkenyl, —C(═O)—NR′—C(═O)—C₁-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₅-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″;
R₁₀ is selected from —H, —C₁-C₂₅alkyl, —C₂-C₂₅alkenyl, —C₂-C₂₅alkynyl, —C₆-C₁₀aryl, each of which being optionally substituted by at least one functionality selected from an hydroxyl, an amine, a halide, —C₁-C₅alkyl, —C₂-C₅alkenyl, —C₂-C₅alkynyl, —C(═O)—, —C(═O)—C₁-C₂₅alkyl, —C(═O)—O—C₁-C₅alkyl, —C(═O)—O—C₁-C₅alkenyl, —C(═O)—O—C₁-C₅alkynyl, —C(═O)—NR′R″R′″, —O—C₁-C₅alkyl, —O—C₁-C₅alkenyl, —O—C₁-C₅alkynyl, —S—, —S—C₁-C₅alkyl, —S—C₁-C₅alkenyl, —S—C₁-C₅alkynyl, —ONO₂, —NO₂, 2,2,6,6-tetramethylpiperidin-1-ol-4-yl, —NHC(═O)CH₂C(CH₃)₂—O-aryl-Cl, idebenonyl-derivative, -pyridine-3-C(═O)—OH and —NR′R″R′″;
each of R′, R″ and R′″ is independently selected from H, C₁-C₅alkyl, C₂-C₅alkenyl, C₂-C₅alkynyl, —C(═O)—C₂-C₂₅alkyl, —C(═O)—C₂-C₂₅alkenyl and C₅-C₂₅alkynyl; or wherein one of R′, R″ and R′″ is absent; and wherein each bond between N-L, L-L₁, L₁-L₂ and L₂-C (designated ---) is a single or double bond.

236. A compound of formula (III): 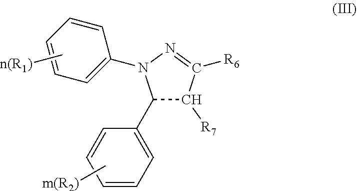
wherein each of R₁, R₂, n, m, R₆ and R₇ is as defined for compound (II); and
wherein --- designates a single or a double bond and wherein, in case it is a double bond, the carbon atom bearing variant R₇ does not carry a bond to a hydrogen atom.

237. A compound of formula (IV): 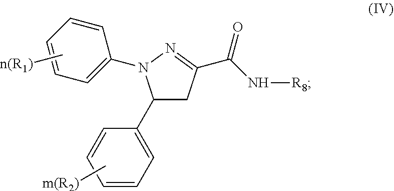
wherein each of R₁, R₂, n, m and R₈ is as defined for compound (II).

238. A compound of the formula (XXVII): 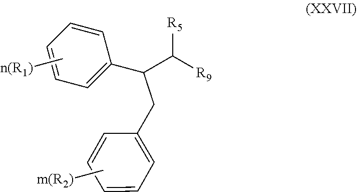
wherein each of R₁, R₂, n, m is as defined herein;
R₅ is absent or selected from H, —C₁-C₃alkyl, —C(═O)—O—R₈, —C(═O)—NR′—R₈, halide, CN, and OH; and R₉ is selected from —C(═O)—O—R₈, —C(═O)—NR′—R₈, —NH—C(═O)—O—R₈, —NH—C(═O)—NR′—R₈, —O—C(═O)—O—R₈ and —O—C(═O)—NR′—R₈; and
where R₈ is as defined herein.

239. A compound of formula (XXX): 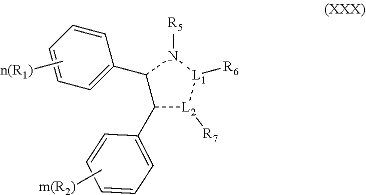
wherein
each of R₁, R₂, n, m, is as defined for compound (XXVII);
one of L₁ and L₂ is a nitrogen atom and the other of L₁ and L₂ is a carbon atom being selected from C, CH or CH₂;
each of R₅, R₆ and R₇, independently of the other, may be is absent or selected from —H, C₁-C₃alkyl, —C(═O)—O—R₈, —C(═O)—NR′—R₈, halide, CN, OH, and NR′R″;
each of R′, and R″ is independently selected from H, C₁-C₅alkyl, C₂-C₅alkenyl, C₂-C₅alkynyl, —C(═O)—C₂-C₂₅alkyl, —C(═O)—C₂-C₂₅alkenyl and C₅-C₂₅alkynyl;
R₈ is as defined for compound (XXVII);
and wherein each bond between C—N, N-L₁, L₁-L₂ and L₂-C (designated ---) is a single bond or double bond.

240. A compound of formula (XXXII): 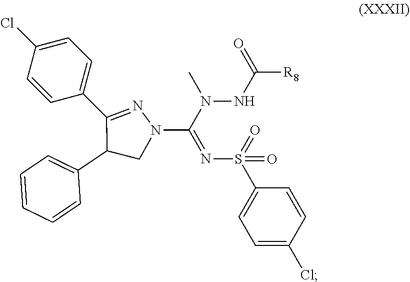
wherein R₈ is as defined for compound (XXVII).

241. A compound of formula (XXXIV): 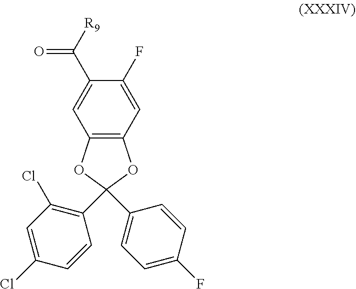
wherein R₉ is selected from —O—R₈ and —NR′—R₈; and wherein R₈ is as defined for compound (II).

242. A compound of formula (XXXV): 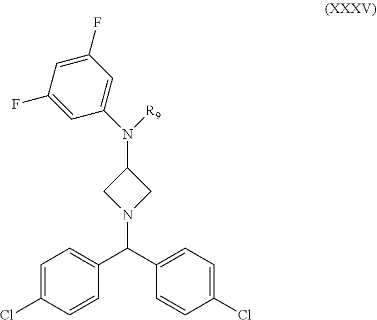
wherein R₉ is selected from —O—R₈ and —NR′—R₈; and wherein R₈ is as defined for compound (II).

243. A compound of formula (XXXVI): 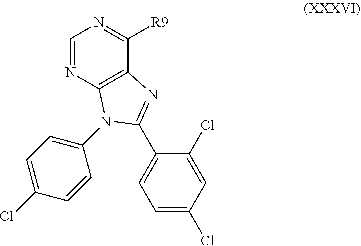
wherein R₉ is selected from —O—R₈ and —NR′—R₈; and wherein R₈ is as defined for compound (II).

244. A compound of formula (XXXX): 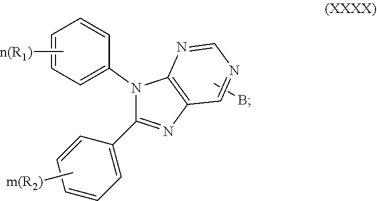
wherein each of R₁, R₂, n, m, is as defined for compound (II); and
B is selected from —H, —C₁-C₂₅alkyl, —C₂-C₂₅alkynyl, —C₆-C₁₀aryl, an hydroxyl, an amine, a halide, —ONO₂, —NO₂, —S—, —S—C₁-C₅alkyl, —S—C₁-C₅alkenyl, —S—C₁-C₅alkynyl, —C(═O)—, —C(═O)—C₁-C₂₅alkyl, —C(═O)—O—C₁-C₅alkyl, —C(═O)—O—C₂-C₅alkenyl, —C(═O)—O—C₂-C₅alkynyl, —C(═O)—NR′R″R′″, —C(═O)—NR′—C(═O)—C₁-C₂₅alkyl, —C(═O)—NR′—C(═O)—C₂-C₂₅alkenyl, —C(═O)—NR′—C(═O)—C₂-C₂₅alkynyl, —C(═O)—OR₁₀, —O—C₁-C₅alkyl, —O—C₁-C₅alkenyl, —O—C₁-C₅alkynyl, —NH—NH₂, —NH—NH—C(═O)—C₁-C₂₅alkyl, —NH—NH—C(═O)—C₂-C₂₅alkenyl, —NH—NH—C(═O)—C₂-C₂₅alkynyl, —NH—NH—C(═O)—C₆-C₁₀aryl, —NH—NH—C(═O)—C₃-C₁₀heteroaryl, —NH—C₁-C₂₅alkyl-C(═O)—OH, —NH—C₂-C₂₅alkenyl-C(═O)—OH, —NH—C₂-C₂₅alkynyl-C(═O)—OH, —NH—C₁-C₂₅alkyl-C(═O)—NR′R″R′″, —NH—C₂-C₂₅alkenyl-C(═O)—NR′R″R′″, —NH—C₂-C₂₅alkynyl-C(═O)—NR′R″R′″, —NH—C₁-C₂₅alkyl-NH₂, —NH—C₂-C₂₅alkenyl-NH₂, —NH—C₂-C₂₅alkynyl-NH₂, —NH—C₁-C₂₅alkyl-NH—C(═O)—C₁-C₂₅alkyl, —NH—C₂-C₂₅alkenyl-NH—C(═O)—C₁-C₂₅alkyl, —NH—C₂-C₂₅alkynyl-NH—C(═O)—C₁-C₂₅alkyl, —NH—C₁-C₂₅alkyl-NH—C(═O)—C₆-C₁₀aryl, —NH—C₂-C₂₅alkenyl-NH—C(═O)—C₆-C₁₀aryl, —NH—C₂-C₂₅alkynyl-NH—C(═O)—C₆-C₁₀aryl, —NH—C₁-C₂₅alkyl-NH—C(═O)—C₃-C₁₀heteroaryl, —NH—C₂-C₂₅alkenyl-NH—C(═O)—C₃-C₁₀heteroaryl, —NH—C₂-C₂₅alkynyl-NH—C(═O)—C₃-C₁₀heteroaryl, —NH—C₁-C₂₅alkylene-C(═O)—NR′R″R′″, —NH—C₂-C₂₅alkenylene-C(═O)—NR′R″R′″, —NH—C₂-C₂₅alkynylene-C(═O)—NR′R″R′″, —NH—C₁-C₂₅alkylene-C(═O)—O—C₁-C₂₅alkyl, —NH—C₂-C₂₅alkenylene-C(═O)—O—C₁-C₂₅alkyl, —NH—C₂-C₂₅alkynylene-C(═O)—O—C₁-C₂₅alkyl, —NHC(═O)C₁-C₂₅alkyl, —NHC(═O)C₂-C₂₅alkenyl, —NHC(═O)C₂-C₂₅alkynyl, —NHC(═O)C₁-C₂₅alkylene-NR′R″R′″, —NHC(═O)C₂-C₂₅alkenylene-NR′R″R′″, —NHC(═O)C₂-C₂₅alkynylene-NR′R″R′″, —NHC(═O)C₁-C₂₅alkylene-OH, —NHC(═O)C₂-C₂₅alkenylene-OH, —NHC(═O)C₂-C₂₅alkynylene-OH, —NHC(═O)C₆-C₁₀aryl, —NHC(═O)C₃-C₁₀heteroaryl, —NHC(═O)C₁-C₂₅alkylene-C₆-C₁₀aryl, —NHC(═O)C₂-C₂₅alkenylene-C₆-C₁₀aryl, —NHC(═O)C₂-C₂₅alkynylene-C₆-C₁₀aryl, —NHC(═O)C₃-C₁₀heteroaryl, —NHC(═O)C₁-C₂₅alkylene-C₃-C₁₀heteroaryl, —NHC(═O)C₂-C₂₅alkenylene-C₃-C₁₀heteroaryl, —NHC(═O)C₂-C₂₅alkynylene-C₃-C₁₀heteroaryl, 2,2,6,6-tetramethylpiperidin-1-ol-4-yl free radical, —NHC(═O)C(CH₃)₂—O-aryl-Cl, —NHC(═O)CH₂C(CH₃)₂—O-aryl-Cl, idebenonyl-derivative, -pyridine-3-C(═O)—OH and —NR′R″R′″.

245. A pharmaceutical composition comprising a compound according to claim 235.

246. A nanocarrier comprising at least one compound of claim 235.

247. A method of preventing or treating a metabolic syndrome and disorders, the method comprises administering to a human or animal subject an amount of a compound of claim 235.

248. The method according to claim 247, wherein the metabolic syndrome or disorders are selected from obesity, insulin resistance, diabetes, coronary heart disease, liver cirrhosis and cancer.

249. A method of treating a subject to reduce body fat, or to reduce body weight, or to treat insulin resistance, or to treat diabetes, or to reduce or control high blood pressure, or to improve a poor lipid profile with elevated LDL cholesterol, low HDL cholesterol, and elevated triglycerides, or to treat a metabolic syndrome, the method comprising administering to the subject at least one compound according to claim 235.
